(19) United States
(12) Patent Application Publication
(10) Pub. No.: US 2020/0224243 A1
(43) Pub. Date: Jul. 16, 2020

(54) Proximity Ligation in Situ Hybridization (PLISH)

(71) Applicant: The Board of Trustees of the Leland Stanford Junior University, Stanford, CA (US)

(72) Inventors: Tushar J. Desai, San Francisco, CA (US); Pehr B. Harbury, Portola Valley, CA (US); Monica Nagendran, San Jose, CA (US); Daniel Riordan, San Francisco, CA (US)

(21) Appl. No.: 16/493,454

(22) PCT Filed: Mar. 22, 2018
(86) PCT No.: PCT/US18/23846

§ 371 Date: Sep. 12, 2019

Related U.S. Application Data
(60) Provisional application No. 62/475,090, filed on Mar. 22, 2017.

(51) Int. Cl. C12Q 1/682; C12Q 1/6888; G16B 40/00; C12N 15/11

(52) U.S. Cl. CPC C12Q 1/682; C12N 15/11; G16B 40/00; C12Q 1/6888

(57) ABSTRACT

Compositions and reagents for molecular profiling using proximity ligation-/>7 situ hybridization (PLISH) are disclosed. In particular, PLISH merges the specificity of proximity ligation, the sensitivity of tiling multiple probes for a target nucleic acid, and the high signal intensity of rolling circle amplification. The probe design capitalizes on the formation of Holliday-like junctions for optimal signal amplification. PLISH provides single molecule resolution and allows for quantitation of a virtually unlimited number of nucleic acids within individual cells. PLISH is also compatible with immunohistochemistry and archival formal-fixed, paraffin-embedded tissue samples.

### TECHNICAL FIELD

The present invention relates to molecular profiling of cells and tissue. In particular, the invention relates to molecular profiling using proximity ligation-in situ hybridization (PLISH).

### BACKGROUND

In parallel with the development of single-cell RNA sequencing (scRNA-seq), there have been rapid advances in single-molecule in situ hybridization (smISH) techniques that localize RNAs of interest directly in fixed cells (Shah et al. (2016) Neuron 92:342-357; Huss et al. (2015) Cold Spring Harbor Protocols 2015:259-268; Chen et al. (2015) Science 348:aaa6090; Wang et al. (2012) The Journal of Molecular Diagnostics 14:22-29; Larsson et al. (2010) Nature Methods 7:395-397, Raj et al. (2008) Nature Methods 5:877-879; Femino et al. (1998) Science 280:585-590). These smISH techniques involve hybridization of fluorescently-labeled oligonucleotide probes, typically 24-96 per gene, to mark individual RNA molecules with a discrete, diffraction-limited punctum that can be quantitatively analyzed by fluorescence microscopy. smISH has been used in cultured cells to study the subcellular distribution of RNAs (reviewed in Buxbaum et al., (2015) Nature Reviews Molecular Cell Biology 16:95-109), the consequences of stochastic noise on gene expression (Raj et al. (2010) Nature 463:913-918; Raj et al. (2006) PLoS Biology 4:e309), and the impact of cell shape and environment on expression programs (Moffitt et al. (2016) eLife 5:e13065; Battich et al. (2015) Cell 163:1596-1610). Frei and colleagues used a different approach to detect multiple transcripts in cells (Frei et al. (2016) Nature Methods 13:269-275). They adopted a strategy whereby two oligonucleotide probes must independently bind in proximity on the target transcript in order to form a scaffold on which subsequent amplification can take place, which integrates high specificity with high signal generation (Wang et al. (2012) Journal of Molecular Diagnostics 14:22-29; Gross-Thebing et al. (2014) BMC Biology 12:55). Their technique utilized classical proximity ligation (Fredriksson et al. (2002) Nature Biotechnology 20:473-477; Soderberg et al. (2006) Nature Methods 3:995-1000) for specificity and Rolling Circle Amplification (RCA) of padlock probes (Larsson et al. (2010) Nature Methods 7:395-397; Ke et al. (2013) Nature Methods 10:857-860) for signal amplification. This approach was suitable for co-detection of multiple transcripts with proteins in single cells by flow or mass cytometry.

An increasingly important application for smISH is the simultaneous localization of customized panels of transcripts in tissue, which is used to validate putative cell subtypes identified by scRNA-seq studies (Grun and van Oudenaarden, (2015) Cell 163:799-810). Performing smISH in intact tissue can also reveal the spatial relationship between the cells expressing secreted signaling factors and the cells expressing the corresponding receptors, information that current scRNA-seq approaches cannot resolve because they require tissue dissociation with irretrievable loss of spatial context. Finally, when applied on a genome-wide scale in tissues, smISH has the potential to entirely bypass scRNA-seq as an upfront discovery tool.

The development of multiplexed smISH for use in tissue has been challenging due to autofluorescent background and light scattering (Shah et al. (2016) Neuron 92:342-357; Sylwestrak et al. (2016) Cell 164:792-804; Moffitt et al. (2016) PNAS 113:14456-14461; Chen et al. (2016) Nature Methods 13:679-684; Choi et al. (2014) ACS Nano 8:4284-4294; Lyubimova et al. (2013) Nature Protocols 8:1743-1758). One strategy for addressing this problem is to amplify probe signals by the hybridization chain reaction (HCR, reviewed in (Choi et al., (2016) Development 143:3632-3637); see also (Wang et al. (2012) The Journal of Molecular Diagnostics 14:22-29) for branched-DNA amplification), which provides up to five orthogonal detection channels. Higher levels of multiplexing can be achieved by repeated cycles of RNA in situ hybridization followed by a re-amplification step (Shah et al. (2016) Neuron 92:342-357), but because a single round of probe hybridization in tissue sections takes hours, multiplexing with HCR is laborious. Unamplified smISH techniques have the practical advantage that hundreds of endogenous RNA species can be barcoded in a single reaction, and then read out with rapid label-image-erase cycles (Moffitt et al. (2016) PNAS 113:14456-14461; Moffitt et al. (2016) PNAS 113:11046-11051), but these do not provide adequate signal in tissues.

Ideally, a technique for high-throughput profiling in tissue would combine all of the RNA probe hybridization and signal amplification steps into a single reaction. Previously, Nilsson and colleagues presented an elegant enzymatic solution to this problem (Larsson et al. (2010) Nature Methods 7:395-397; Ke et al. (2013) Nature Methods 10:857-860). They used barcoded padlock probes to label cDNA molecules in cells and tissues, and rolling-circle amplification (RCA) to transform the circularized probes into long tandem repeats. The approach worked in tissues and handled an unbounded number of orthogonal amplification channels. The only limitations were that the RNA-detection efficiency was capped at about 15% (each transcript could only be probed at a single site because the 3′ end of the cDNA served as the replication primer), and that the approach required an in situ reverse transcription step with specialized and costly locked nucleic-acid primers.

Thus, better methods are needed for molecular profiling of cells and tissue.

### SUMMARY

The invention relates to reagents and methods for detecting nucleic acids using proximity ligation-in situ hybridization (PLISH). PLISH utilizes probes, which bind along the length of each target nucleic acid and rolling circle amplification (RCA) to increase the signal for detection. A key feature endowing PLISH with ultrasensitive transcript detection is the oligonucleotide probe design that results in formation of Holliday-like junctions. Specificity is achieved by incorporating proximity ligation, wherein production of a detectable signal depends on binding of at least two probes sufficiently close together on a nucleic acid to allow ligation to produce circular DNA for amplification. Random and even sequence-specific off-target binding of a single probe does not produce a signal. PLISH is compatible with automated image analysis for multiplex expression profiling of large numbers of single cells.

In one aspect, the invention includes a method of detecting one or more target nucleic acids in a sample, the method comprising: a) providing at least one probe set for each target nucleic acid, wherein each probe set comprises: i) a first probe comprising a 5′ overhang region and a region that hybridizes to the target nucleic acid at a first target site; ii) a second probe comprising a 3′ overhang region and a region that hybridizes to the target nucleic acid at a second target site; b) contacting the sample with the probe sets; c) adding at least one bridge oligonucleotide to the sample for each probe set, wherein the bridge oligonucleotide comprises i) a first portion that hybridizes to a complementary portion in the 5′ overhang region of the first probe of the probe set, and ii) a second portion that hybridizes to a complementary portion in the 3′ overhang region of the second probe of the probe set, wherein the first probe and the second probe, when bound to one of the target nucleic acids, are in sufficient proximity to each other to simultaneously hybridize to the bridge oligonucleotide; d) adding at least one circle oligonucleotide to the sample for each probe set, wherein the circle oligonucleotide comprises a first portion that hybridizes to a complementary region at the 5′ end of the 5′ overhang region of the first probe of the probe set, and a second portion that hybridizes to a complementary region at the 3′ end of the 3′ overhang region of the second probe of the probe set; e) forming circular DNA where any two probes of a probe set bind sufficiently close to each other on one of the target nucleic acids to allow ligation of the bridge oligonucleotide and circle oligonucleotide that are hybridized to the two probes to generate a closed circle; f) performing rolling circle amplification, wherein each circular DNA molecule formed serves as a template to produce a concatemer comprising multiple copies of the circular DNA nucleotide sequence; g) contacting each concatemer with one or more imager oligonucleotides, wherein each imager oligonucleotide comprises a detectable label and a nucleotide sequence complementary to one or more sites in the circular DNA sequence, wherein the imager oligonucleotide binds to said sites in the multiple copies of the circular DNA sequence of the concatemer; and h) detecting the bound imager oligonucleotides.

In certain embodiments, the first target site is located either 5′ of the second target site or 3′ of the second target site on the target nucleic acid. In certain embodiments, the first and second target sites are adjacent to each other on the target nucleic acid, or the first and second target sites are contiguous on the target nucleic acid.

In another embodiment a plurality of probe sets comprising probes capable of hybridizing at a plurality of target sites on a single target nucleic acid are used.

In another embodiment, a plurality of probe sets comprising probes capable of hybridizing at a plurality of target sites on multiple target nucleic acids are used for multiplexed detection of a plurality of target nucleic acids. The method may further comprise using a plurality of circle oligonucleotides, wherein each circle oligonucleotide binds to a different probe set; and a plurality of imager oligonucleotides, wherein each imager oligonucleotide comprises a different detectable label. For example, each circle oligonucleotide may comprise one or more binding sites for a different imager oligonucleotide, such that different circle oligonucleotides are bound by different imager oligonucleotides comprising different detectable labels to allow different target nucleic acids to be detectably distinguished from one another.

Exemplary detectable labels include fluorescent labels, bioluminescent labels, chemiluminescent labels, isotopic labels, nanoparticles, and metals.

In certain embodiments, each probe has a similar melting temperature (Tₘ) for binding to its cognate target site. For example, the Tₘ may range from about 45° C. to about 65° C., including any Tₘ within this range such as 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C.

In certain embodiments, the target nucleic acids are RNA or DNA. For example, a target nucleic acid may be an RNA selected from the group consisting of a messenger RNA, a ribosomal RNA, a transfer RNA, a non-coding RNA, and a regulatory RNA.

In certain embodiments, a bridge oligonucleotide or circle oligonucleotide comprises at least one binding site for an imager oligonucleotide. In other embodiments, the bridge and circle oligonucleotides both comprise at least one binding site for an imager oligonucleotide. In another embodiment, a circle oligonucleotide comprises multiple binding sites for an imager oligonucleotide.

In certain embodiments, the target nucleic acids are in a cell. The cell may be a eukaryotic cell (e.g., an animal cell, a plant cell, a fungal cell, or a protist cell), a prokaryotic cell, an archaeon cell, or an artificial cell. In another embodiment, the cell is a human cell. The cell may be a fixed cell or a live cell. In another embodiment, the method further comprising lysing or permeabilizing the cell.

In certain embodiments, the target nucleic acids are in a population of cells, a tissue, an organ, or an organism. For example, methods of the invention may be performed on a sample comprising a plurality of cell types, such as a biopsy or blood sample potentially including immune cells, progenitor or stem cells, or cancer cells. In certain embodiments, the method further comprises mapping an anatomical location for at least one target nucleic acid in a tissue or organ.

In certain embodiments, a cell or tissue is exposed to a test condition prior to said contacting the sample with one or more probe sets. For example, the test condition may comprise exposing a cell or tissue to a drug, a ligand for a receptor, a hormone, a second messenger, a pathogen, a genetic modification, a change in temperature, growth media, membrane potential, or osmotic pressure.

In certain embodiments, a subset of the target nucleic acids is detected simultaneously.

In certain embodiments, the detectable labels on the imager oligonucleotides are fluorescent labels. Such labels can be detected, for example, by performing fluorescence imaging. In some embodiments, multiple cycles of fluorescence imaging are performed to allow detection of subsets of the target nucleic acids sequentially.

In another embodiment, subsets of the target nucleic acids are detected sequentially by a method comprising: a) contacting the sample with a subset of the imager oligonucleotides; b) performing a cycle of fluorescence imaging; c) removing the imager oligonucleotides from the sample; d) contacting the sample with another subset of the imager oligonucleotides; e) performing another cycle of fluorescence imaging; and f) removing the imager oligonucleotides from the sample. The method may further comprise repeating steps (a)-(f) until all of the imager oligonucleotides have been used for detection of the plurality of target nucleic acids.

In another embodiment, the method further comprises sequencing at least one target nucleic acid.

In another embodiment, the method further comprises detecting at least one protein in the sample. For example, the method may further comprise performing immunohistochemistry on the sample.

In certain embodiments, a plurality of cell types is present in the sample. In another embodiment, the method further comprises identifying at least one cell type based on detection of one or more target nucleic acids. In some embodiments, the identification of cell types is automated by using an algorithm for cell classification, such as a clustering algorithm (e.g., K-means clustering) or a machine learning algorithm (e.g., t-distributed stochastic neighbor embedding).

In another aspect, the invention includes a composition for detecting one or more target nucleic acids in a sample comprising: a) at least one probe set for each target nucleic acid, wherein each probe set comprises: i) a first probe comprising a 5′ overhang region and a region capable of hybridizing to the target nucleic acid at a first target site; ii) a second probe comprising a 3′ overhang region and a region capable of hybridizing to the target nucleic acid at a second target site; b) at least one bridge oligonucleotide for each probe set, wherein the bridge oligonucleotide comprises i) a first portion capable of hybridizing to a complementary portion in the 5′ overhang region of the first probe of the probe set, and ii) a second portion capable of hybridizing to a complementary portion in the 3′ overhang region of the second probe of the probe set, wherein the first probe and the second probe, when bound to one of the target nucleic acids, are in sufficient proximity to each other to simultaneously hybridize to the bridge oligonucleotide; and c) at least one circle oligonucleotide for each probe set, wherein the circle oligonucleotide comprises a first portion capable of hybridizing to a complementary region at the 5′ end of the 5′ overhang region of the first probe of the probe set, and a second portion capable of hybridizing to a complementary region at the 3′ end of the 3′ overhang region of the second probe of the probe set.

In another aspect, the invention includes a kit comprising any of the compositions described herein and instructions for detecting target nucleic acids. The kit may further comprise other reagents for detecting target nucleic acids, as described herein, such as a ligase and/or reagents for performing rolling circle amplification (e.g., a polymerase, deoxyribonucleotides).

In another aspect, the invention includes an oligonucleotide comprising a nucleotide sequence selected from the group consisting of SEQ ID NOS:1-464, or sequences displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto.

In another aspect, the invention includes a system for performing multiplex detection of target nucleic acids comprising a hybridization chamber sealed to a solid support, such as a coverslip or slide supporting a cell or tissue sample. Multiplex assays are performed by stepwise application of the oligonucleotide reagents, including the probes, circle oligonucleotides, bridge oligonucleotides, and imager oligonucleotides through an inlet port to the hybridization chamber. Oligonucleotide reagents travel through an outlet port of the hybridization chamber to contact cells or tissue on the solid support.

The methods of the invention may be combined with any other method for measuring cellular parameters, including but not limited to immunostaining, immunohistochemistry, mass cytometry, or fluorescence activated cell sorting (FACS), or any other method that can be used to characterize a cell subpopulation of interest (e.g., by detection of cellular markers such as protein markers that differentiate different cell types of interest). Quantification of detection probes may be used to determine the abundances of target nucleic acids and may be used to identify cells expressing the target nucleic acids at different levels.

These and other embodiments of the subject invention will readily occur to those of skill in the art in view of the disclosure herein.

### BRIEF DESCRIPTION OF THE FIGURES

FIGS. 1A-1G show RNA detection by proximity ligation at RNA-DNA Holliday junctions. FIG. 1A shows the mechanism of RNA detection by PLISH. Left (HL) and right (HR) DNA ‘H’ probes targeting adjacent sites hybridize to a target RNA. Subsequent addition of circle and bridge oligonucleotides harboring a specific ‘barcode’ sequence (gray dash) results in an RNA-DNA Holliday junction. The nicks in the junction are then sealed by ligation to create a covalently closed circle, and rolling-circle amplification (RCA) generates complementary tandem repeats. The single-stranded amplicons are detected with a fluorescently-labeled ‘imager’ oligonucleotide (red star) that is complementary to the specific ‘barcode’ sequence. See note in 1C. FIG. 1B shows that to increase efficiency of detection for low abundance transcripts, H probe pairs were embedded with the same barcode sequence can be ‘tiled’ along the length of the target mRNA. FIG. 1C shows that up to five distinct transcripts can be simultaneously detected using five different barcode sequences (one unique sequence for each RNA), and five complementary imager oligonucleotides that are conjugated to spectrally-distinct fluorophores. FIG. 1D shows that PLISH RNA detection requires coincident hybridization of both left and right H probes, and redundant probe sets increase detection efficiency. Cultured HCT116 cells were stained with one (left panel) or ten (middle panel) pairs of H probes targeting the SOX4 transcript. Many more RNA molecules are detected with ten probe sets. No puncta are observed when the RNA-recognition sequences of the left H probes are scrambled (right panel). SOX4, SRY-box 4; Scale bar, 10 mm. FIG. 1E shows that PLISH RNA detection in tissues is highly sequence-specific. Mouse lung was hybridized with a single pair of H probes targeting nucleotides 228-268 of the Scgb1a1 transcript. The section in the bottom row was pre-incubated with a 60-base antisense blocking oligonucleotide complementary to nucleotides 219-278, whereas the section in the top row was pre-incubated with a scrambled 60-base blocking oligonucleotide. The antisense blocking oligonucleotide dramatically reduces the Scgb1a1 signal (bottom), whereas the scrambled blocking oligonucleotide has no effect (top). Note that the PLISH signal is tightly restricted to the bronchial Club cells (arrow). The dashed lines indicate the basal surface of bronchial epithelium. Scgb1a1, secretoglobin family 1A1 member 1; Scale bar, 40 mm. FIG. 1F shows that PLISH RNA detection sensitivity in cultured cells matches single-cell qPCR sensitivity. FPKM values for 36 mRNAs are plotted against the fraction of HCT116 cells in which they were detected by single-cell qPCR (filled black circles) or by PLISH (red inverted triangles). The black line is the prediction of a Poisson sampling model for the fraction of cells with at least one transcript, assuming that the transcript abundance increases proportionately with FPKM, and that one FPKM unit corresponds to 2.5 copies per cell. The inset shows PLISH staining for CASP9, which has an FPKM value of 2. CASP9, caspase 9; FPKM, Fragments Per Kilobase of transcript per Million mapped reads; qPCR, quantitative reverse transcription polymerase chain reaction; Scale bar, 20 mm. (FIG. 1G) RNA abundances measured by PLISH and by single-cell RNA sequencing are highly correlated. A log-log plot showing the average single-cell FPKM value for 10 mRNAs in HCT116 cells plotted against the number of puncta per cell per probe measured by PLISH. Multiple points at each FPKM value are independent experiments. The data fit to a line of slope 1 with R²=0.8.

FIGS. 2A-2F show direct visual analysis of single-molecule and single-cell gene expression in diverse specimens. FIG. 2A shows the PLISH experimental workflow. After an initial probe hybridization and enzymatic amplification step, up to five distinct channels can be simultaneously detected and imaged by conventional fluorescence microscopy, enabling direct visualization of RNA abundance. FIG. 2B shows that PLISH detects single RNA molecules with single-cell resolution in tissues. PLISH staining for Foxj1 and Scgb1a1 in the bronchial epithelium of mouse lung shows a single ciliated cell (Foxj1⁺, arrowhead and asterisk) between Club cells (Scgb1l1⁺) in a planar view (top) and with orthogonal reconstruction (bottom). Note the discrete white puncta in the ciliated cell, which correspond to single Foxj1 transcripts. Dashed lines indicate the lateral and solid lines indicate the basal surface of airways. Foxj1, forkhead box J1; Scgb1a1, secretoglobin family 1A member 1; Scale bars, 10 mm. FIG. 2C shows simultaneous RNA and protein detection in FFPE sections. FFPE human lung co-stained by PLISH (SCGB1A1, red) and indirect immunohistochemistry (anti-KRT5, grey) shows the expected localization of Club cells (SCGB1A1⁺, arrow) and basal cells (KRT5⁺, arrowhead) along the bronchial (Br) epithelium. Solid lines indicate the basal surface of airways. FFPE, formalin-fixed, paraffin-embedded; KRT5, keratin 5; SCGB1A1, secretoglobin family 1A member 1; Scale bar, 5 mm. FIG. 2D shows discrimination of AT2 cells from macrophages by visual inspection of RNA abundance. PLISH staining in mouse lung for Lyz2 and Sftpc allows clear discrimination of alveolar macrophages (Lyz2⁺ Sftpc⁻, arrow) from AT2 cells (Sftpc⁺ Lyz2⁺, arrow). AT2, Alveolar epithelial type II; Lyz2, lysozyme 2; Mac, macrophage; Sftpc, surfactant protein C; Scale bar, 20 mm. FIG. 2E shows discrimination of AT2 cells from BASC cells by visual inspection of RNA abundance. PLISH staining for the Club cell marker (Scgb1a1) and AT2 cell marker (Sftpc) shows AT2 (Sftpc⁻), Club (Scgb1a1⁺) and BASC (Sftpc⁺ Scgb1a1^{Lo}) cells. Note the discrete red puncta in the ‘BASC’ cells, which correspond to single Scgb1a1 transcripts. The cell types localize appropriately, with the AT2 cell in an alveolus (arrow), the Club cells in the terminal bronchiole, and the double-positive BASCs at the bronchioalveolar junction (arrows). Dashed lines demarcate the airway. AT2, alveolar epithelial type II; BASC, bronchioalveolar stem cell; Scgb1a1, secretoglobin family 1A member 1; Sftpc, surfactant protein C; Scale bar, 20 mm. FIG. 2F shows PLISH in patient tissue samples for molecular analysis of human disease. PLISH staining for SFTPC in non-IPF human lung (left) marks AT2 cells (white arrow) distributed within alveolar septae (dashed lines). The adjacent panels show a magnified image of healthy cuboidal AT2 cells (dashed circles). PLISH staining in IPF human lung (right) shows densely cellular regions with architectural distortion of alveolar septae (dashed lines). SFTPC^{Hi} AT2 cells are inappropriately clustered (white arrowheads) and have abnormal flattened morphologies, as seen at higher magnification in right panels (dashed ellipses). AT2, alveolar epithelial type II; IPF, Idiopathic Pulmonary Fibrosis; SFTPC, surfactant protein C; Scale bar, 100 mm.

FIGS. 3A-3D show multiplexed PLISH: rapid label-image-erase cycles, automated data analysis, and unsupervised cell classification. FIG. 3A shows the multiplexed PLISH experimental workflow. Probes for many different RNAs are hybridized and amplified in a single reaction. The PLISH amplicons marking four RNA species are then labeled with four fluorescent imager oligonucleotides, imaged on a microscope, and ‘erased’ by elimination of the imager oligonucleotides. Amplicons marking a different subset of four RNAs are then labeled with four new imager oligonucleotides, imaged, and erased. This cycle is repeated until all of the RNA species have been visualized and photo-documented. The images are automatically aligned and processed, and the signal for each RNA species in each cell is summed to produce single-cell expression profiles. Unsupervised k-means clustering of the expression profiles (or other computational tools) empirically identifies distinct cell classes. t-SNE plots (or other data visualization tools) show the differences in gene expression and the classification for all of the cells in a tissue. The location of individual cells or cell classes is spatially remapped onto images of the tissue in order to integrate molecular, histological, and spatial features. FIG. 3B shows a multiplexed PLISH data set. Eight different transcripts in mouse lung were visualized with two label-image-erase cycles. A micrograph for each channel in one field of view is shown. Solid lines indicate the basal surface of airways and dashed lines indicate alveolar septae. Actb, beta actin; Ager, advanced glycosylation end product-specific receptor; Ftl1, ferritin light polypeptide 1; Gapdh, glyceraldehyde-3-phosphate dehydrogenase; Lyz2, lysozyme 2; Scgb1a1, secretoglobin family 1A member 1; Sftpc, surfactant protein C; Xist, inactive X specific transcripts. Scale bar, 80 mm. FIG. 3C shows automated cell classification. K-means clustering partitions −2900 single cells into one of ten molecularly distinct classes, with the expression profile of each cluster centroid displayed in a heat map. Based on marker gene expression, individual clusters were inferred to be macrophages (mac), two classes of AT2 cells, two classes of Club cells, AT1 cells, and four categories of unassigned cell types (shades of blue, purple, and orange). FIG. 3D shows an overview of the cells in a murine lung. Differences in gene expression for −2900 cells are displayed as a two-dimensional t-SNE plot. Each cell is represented by a single dot, colored according to its cluster assignment. Labels mark the location of each cell class. The arrowhead indicates a small island of cells that exhibit the profile of BASCs. AT1, alveolar epithelial type I cell; AT2, alveolar epithelial type II cell; BASC, bronchioalveolar stem cell; Mac, macrophage.

FIGS. 4A-4J show biological insights from integrated molecular and spatial information. FIGS. 4A-4D show specificity and promiscuity in marker gene expression. t-SNE plots, which are colored according to the expression of four cell-type marker genes. High expression (light gray) in the first two panels highlights AT2 (FIG. 4A) and Club cells (FIG. 4B), as indicated, while the arrowhead indicates rare double-positive BASCs. FIG. 4C shows high levels of Ager in AT1 cells (arrow), but promiscuous expression in a subset of AT2, Club¹ and Other^{b} cell classes (gray arrowheads). Lyz2 expression in the fourth panel (FIG. 4D) is restricted to the macrophage and AT2¹ classes. Note that differential Lyz2 expression splits the canonical AT2 cell type into two sub-classes (AT2¹ and the AT2²). FIGS. 4E-4F show differential expression of ‘housekeeping’ genes in canonical cell types. t-SNE plots are colored according to the expression of three ubiquitous ‘housekeeping’ genes. Gapdh (FIG. 4E) is the most evenly and broadly distributed, while Ftl1 (FIG. 4F) is highest in the macrophage and Club cell classes. Actb is the highest in the macrophage and AT1 cell classes. FIG. 4G shows that unexpectedly, differential Actb expression splits the canonical Club cell type into two sub-classes (Club¹ and Club²). FIG. 4H shows spatial organization of lung cell classes. The nuclei of cells in a transmitted light image of a bronchioalveolar duct junction (BADJ) are pseudocolored according to their basic cluster assignment. The Club class localizes to the bronchial epithelium, while the AT1 and AT2 cell classes are distributed throughout the alveolar compartment. The macrophage class (white) is primarily found in the alveolar lumen. Rare BASCs (light gray) localize precisely to the bronchioalveolar junctions (arrow), where they have been shown to reside by immunostaining (FIG. 4H and FIG. 9E). This image demonstrates how PLISH can be used to localize specific cells of interest within their anatomical context. Solid lines indicate the basal surface of airways and dashed lines demarcate alveolar septae. Scale bar, 80 mm. FIG. 4I shows spatial organization in the terminal airway. The nuclei in three terminal airway fields of view are pseudocolored according to their cluster assignment. Note the presence of both Club¹ and Club² cell classes, and all four Other cell classes. The Other^{d} class is enriched in pulmonary arteries, indicated by black dashed lines, and therefore might represent endothelial or perivascular cells. Solid lines indicate the basal surface of airways, black dashed lines indicate pulmonary arteries, and dashed white lines demarcate alveolar septae. Ar, artery; Scale bar, 80 mm. FIG. 4J shows two subclasses of Club cells, defined by a difference in Actb expression, segregate anatomically. Club cells in the three fields of view from panel FIG. 4I are pseudocolored with enhanced contrast, revealing a striking spatial pattern. The Club¹ sub-class (Actb^{Hi} Ager^{Lo}) localizes to the BADJ, whereas the Club sub-class (blue, Actb^{Lo}Ager⁻) segregates more proximally in the terminal airways. Differential Actb expression might reflect region-specific differences in mechanical stress. The integration of molecular and spatial information in this image reveals biology that would be missed with either piece of information alone. Solid lines indicate the basal surface of airways, black dashed lines indicate pulmonary arteries, and dashed white lines demarcate alveolar septae. Ar, artery; BADJ, bronchioalveolar duct junction; Scale bar, 80 mm.

FIGS. 5A-5F show the signal-to-noise in PLISH images. FIG. 5A shows an unprocessed micrograph of a mouse lung interrogated with H probes targeting Scgb1a1 and DAPI. The line demarcates a region of the micrograph used to measure the background signal. Scale bar, 50 mm. FIG. 5B shows a plot of pixel intensities in the red channel from the image in FIG. 5A. The intensities ranged from 0 to 255. FIG. 5C shows a zoomed-in histogram of the red pixel intensities within the field demarcated by the line, which was used to measure the background signal. 435,175 of these 435,200 background pixels had 0 counts (the vertical axis is truncated), and the mean intensity was 1.3×10⁻⁴ counts. FIG. 5D shows a histogram of the pixel intensities for all the non-zero pixels in FIG. 5A. The mean intensity of the non-zero pixels was 42 counts. FIG. 5E shows a micrograph of PLISH puncta for PP1A and DAPI in cultured HCT116 cells. PP1A, protein phosphatase 1A; Scale bar, 10 mm. FIG. 5F shows a histogram of the integrated intensities for the PLISH puncta in FIG. 5E (filled circles). The histogram fitted well to a negative binomial distribution with a single ‘fail’ event (open circles). Mean-events-to-failure parameters between 1000 and 60,000 gave similar agreement with the data.

FIG. 6 shows benchmarking PLISH specificity against a validated antibody by co-staining in tissue. Mouse lung co-stained for Sftpc by indirect immunohistochemistry and PLISH with DAPI. 181 of 184 PLISH+ cells were also antibody+, and 181 of 195 antibody+ cells were PLISH+. White arrows indicate representative co-labeled AT2 cells. Dotted lines demarcate alveolar septae. AT2, alveolar epithelial type II; Scale bar, 40 mm.

FIG. 7 shows estimate of efficiency of PLISH probes in tissue. Double in situ hybridization for Axing using HCR and PLISH was performed in mouse lung. HCR signals (arrowheads, third panel) were identified based on overlap of puncta from two different HCR channels, and PLISH puncta were imaged in the same field (white arrowheads, fourth panel). Colocalized HCR and PLISH puncta are enumerated (dashed orange circles) in the fifth panel. Over three fields of view, we observed 92 HCR puncta and 140 PLISH puncta, with 29 cases of co-localized HCR and PLISH signal. Thus, the four PLISH probe pairs gave a combined detection efficiency of 32%, with a per-site efficiency of 9%. Dashed line demarcates alveolar septae. Scale bar, 40 mm.

FIGS. 8A-8C show rapid label-image-erase strategies without tissue degradation. FIG. 8A shows that imaging with or without washout of the fluorophore-labeled ‘imager’ oligonucleotides gives identical signal-to-noise ratios. Mouse lung tissue sections were interrogated with a single PLISH probe pair targeting Sftpc. In the top panel, the tissue section was hybridized with 100 nM imager oligonucleotides then washed (to remove excess imager oligonucleotides) prior to imaging, which took 30-60 minutes. In the bottom panel, the tissue section was hybridized with 3 nM imager oligonucleotides then imaged without a wash step, taking only 5 minutes. Bronchioles indicated by dashed lines. Sftpc, surfactant protein C; Scale bar, 40 mm. FIG. 8B shows signal erasure by dissociation of short imager oligonucleotides. PLISH puncta for Sftpc were visualized with short (11 base pair) imager oligonucleotides (first column). The signal was then erased by washing at 37° C. for 15 minutes and re-imaged (middle column), showing no residual fluorescence. The sample was then re-stained with short imager oligonucleotides and re-imaged (third column), showing re-emergence of the fluorescent signal. The cycle time was 25 minutes. Bronchioles indicated by dashed lines. Sftpc, surfactant protein C; Scale bar, 40 mm. FIG. 8C shows signal erasure by enzymatic digestion of imager oligonucleotides. PLISH puncta for Xist were visualized in mouse lung with uracil-containing imager oligonucleotides (left panel). The tissue section was then incubated with the New England Biolabs USER enzyme cocktail at 37° C. for 20 minutes to digest the imager oligonucleotides and re-imaged (middle panel), then re-stained with a new set of imager oligonucleotides for Ager (right panel). The cycle time was 25 minutes. Ager, advanced glycosylation end product-specific receptor; Xist, inactive X specific transcripts; Scale bar, 20 mm.

FIGS. 9A-9E show immunohistochemical and single cell RNA-sequencing correlation of PLISH results. FIG. 9A shows Lyz2^{-/EGFP} mouse lung stained with anti-GFP to mark Lyz2⁻ cells, anti-Sftpc (AT2), and anti-Cdh1 (epithelial) discriminates three cell populations. Macrophages (arrowhead, Lyz2⁺ Sftpc⁻Cdhl⁻), AT2¹ (Sftpc⁺ Lyz2⁺ Cdh1⁺) and AT2² (Sftpc⁺ Lyz2⁻ Cdh1⁺ ). AT2 cells indicated by arrows. Dashed line indicates basal surface of bronchiole. AT2, alveolar epithelial type II; Mac, macrophage; Cdh1, cadherin 1; Lyz2, lysozyme 2; Sftpc, surfactant protein C; Scale bar, 40 mm. FIG. 9B shows anti-Ager labels the apical surface of cells in the bronchial epithelium (arrowheads). Dashed lines indicate the basal surface of airways. Ager, advanced glycosylation end product-specific receptor; BADJ, bronchioalveolar duct junction; Scale bar, 20 mm. FIG. 9C shows heat-map of single cell RNA-sequencing of Club (cyan bar) and AT2 (bar) cells shows low expression of Ager in a subset of cells from both populations, supporting demonstration by PLISH of promiscuous expression of this AT1 cell marker. Note also broad expression of Lyz2 by AT2 cells (see t-SNE of Ager and Lyz2 in FIG. 4A). AT1, alveolar epithelial type I; AT2, alveolar epithelial type II; Ager, advanced glycosylation end product-specific receptor; Lyz2, lysozyme 2. FIG. 9D shows mouse lung co-stained for known (Ager) and novel (Akap5) AT1 markers by PLISH. Arrowheads indicate representative Ager^{Hi} AT1 cells also expressing Akap5. Area in dashed box shown enlarged in right panels. Dashed lines demarcate alveolar septae. AT1, alveolar epithelial type I; Ager, advanced glycosylation end product-specific receptor; Akap5, A-Kinase Anchoring Protein 5; Scale bars, 50 mm. FIG. 9E shows mouse lung stained with anti-Scgb1a1 and anti-Sftpc shows double positive BASCs (arrowheads) localized to the BADJ. Dashed lines indicate the basal surface of airway epithelium; BADJ, bronchioalveolar duct junction; Scgb1a1, secretoglobin family la member 1; Sftpc, surfactant protein C; Scale bar, 40 mm.

### DETAILED DESCRIPTION

The practice of the present invention will employ, unless otherwise indicated, conventional methods of chemistry, cell biology, biochemistry, molecular biology and recombinant DNA techniques, and immunology within the skill of the art. Such techniques are explained fully in the literature. See, e.g., RNA: Methods and Protocols (Methods in Molecular Biology, edited by H. Nielsen, Humana Press, 1^{st} edition, 2010); Rio et al. RNA: A Laboratory Manual (Cold Spring Harbor Laboratory Press; 1st edition, 2010); Farrell RNA Methodologies: Laboratory Guide for Isolation and Characterization (Academic Press; 4^{th} edition, 2009); PCR Technology: Current Innovations (T. Nolan and S. A. Bustin eds., CRC Press, 3^{rd} edition, 2013); Antibodies A Laboratory Manual (E. A. Greenfield ed., Cold Spring Harbor Laboratory Press, 2^{nd} Lab edition, 2013); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a probe” includes two or more probes, and the like.

The term “about,” particularly in reference to a given quantity, is meant to encompass deviations of plus or minus five percent.

As used herein, a “cell” refers to any type of cell from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. A cell may include a fixed cell, permeabilized cell, or a live cell. The methods described herein can be performed, for example, on a sample comprising a single cell, a population of cells, or a tissue or organ.

A “live cell,” as used herein, refers to an intact cell, naturally occurring or modified. The live cell may be isolated from other cells, mixed with other cells in a culture, or within a tissue (partial or intact), or an organism.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

As used herein, a “solid support” refers to a solid surface such as a magnetic bead, latex bead, microtiter plate well, glass plate, nylon, agarose, acrylamide, and the like.

“Substantially purified” generally refers to isolation of a substance (e.g., compound, nucleic acid, oligonucleotide, protein, or peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a protein, polypeptide or peptide, that the indicated molecule is separate and discrete from the whole organism with which the molecule is found in nature or is present in the substantial absence of other biological macro molecules of the same type. The term “isolated” with respect to a nucleic acid is a nucleic acid molecule devoid, in whole or part, of sequences normally associated with it in nature; or a sequence, as it exists in nature, but having heterologous sequences in association therewith; or a molecule disassociated from the chromosome.

As used herein, the term “target nucleic acid region” or “target nucleic acid” denotes a nucleic acid molecule with a “target sequence” to be detected or amplified. The target nucleic acid may be either single-stranded or double-stranded and may include other sequences besides the target sequence. The term “target sequence” or “target site” refers to the particular nucleotide sequence of the target nucleic acid which is detected by binding of a probe. The target sequence may include a probe-hybridizing region contained within the target molecule with which a probe will form a stable hybrid under desired conditions. The “target sequence” may also include the sequences to which oligonucleotide primers complex and are extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and anti-sense strands).

The term “adjacent” or “substantially adjacent” as used herein refers to the positioning of two regions or target sites on the target nucleic acid. The two adjacent regions or target sites (e.g., where a pair of probes bind) may be separated by 0 up to 150 nucleotides, including any number of nucleotides in this range such as 0, 1, 2, 3, 4, 5, 6, 7, 8, 9,10, 15, 20, 25, 30, 35, 40, 45, 50, 55, 60, 65, 70, 75, 80, 85, 90, 100, 110, 120, 130, 140, or 150 nucleotides. A zero nucleotide gap means that the two regions or target sites directly abut one another. In other words, the two regions bound by a pair of probes may be contiguous, i.e. there is no gap between the two target sites. Alternatively, the two regions hybridized by the oligonucleotides may be separated by 1 to about 150 nucleotides.

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

The term “amplicon” refers to the amplified nucleic acid product of a polymerase chain reaction (PCR), rolling circle amplification (RCA), or other nucleic acid amplification process.

As used herein, the term “probe” or “oligonucleotide probe” refers to a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The terms “selectively detects” or “selectively detecting” refer to the detection of a nucleic acid (e.g., DNA or RNA transcript) using oligonucleotides (e.g., probes, circle oligonucleotides, and bridge oligonucleotides) that are capable of detecting a particular target sequence, for example, by amplifying and/or binding to a target sequence of a particular nucleic acid, or ligation product or extension product thereof, but do not amplify and/or bind to other nucleic acid sequences under appropriate hybridization conditions.

As used herein, the term “detectable label” refers to a molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647,and Alexa Fluor 784, a cyanine dye such as Cy 3, Cy3.5, Cy5, Cy5.5, and Cy7, fluorescein, 2′,4′,5′,7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neo®, G418′) dihydrofolate reductase (DHFR), hygromycin-B-phosphotransferase (HPH), thymidine kinase (TK), β-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. The term also includes chemiluminescent labels such as luminol, isoluminol, acridinium esters, and peroxyoxalate and bioluminescent proteins such as firefly luciferase, bacterial luciferase, Renilla luciferase, and aequorin. The term also includes isotopic labels, including radioactive and non-radioactive isotopes, such as, ³H, ²H, ¹²⁰I , ¹²³I, ¹²⁴I, ¹²⁵, ¹³¹I , ³⁵S, ¹¹C, ¹³C, ¹⁴C, ³²P, ¹⁵N, ¹³N, ¹¹⁰I, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ⁵²Mn, ⁵⁵Co, ⁷²As, ⁷⁵Br, ⁷⁶Br, ^{82m}Rb, and ⁸³Sr. The term also includes color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.), near infrared (NIR) probes, and nanoshells. The term also includes contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

The term “subject” or “host subject” includes bacteria, archaea, fungi, protists, plants, and animals (both vertebrates and invertebrates), including, without limitation, plants such as flowering plants (e.g., Arabidopsis thaliana), conifers and other gymnosperms, ferns, clubmosses, hornworts, liverworts, mosses (e.g., Physcomitrella patens), and green algae (e.g., Chlamydomonas reinhardtii); fungi such as molds and yeasts (e.g., Saccharomyces cerevisiae, Schizosaccharomyces pombe), protists such as amoebae, flagellates, and ciliates (e.g., Tetrahymena thermophila); worms (e.g., Caenorhabditis elegans), insects such as beetles, ants, bees, moths, butterflies, and flies (e.g., Drosophila melanogaster), amphibians such as frogs (e.g., Xenopus tropicalis, Xenopus laevis) and salamanders (e.g., axolotls); fish (e.g., Danio rerio, Fundulus heteroclitus, Nothobranchius furzeri); reptiles; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

As used herein, a “biological sample” refers to a sample of cells, tissue, or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, cells, muscles, joints, organs, biopsies, and also samples of in vitro cell culture constituents including but not limited to conditioned media resulting from the growth of cells and tissues in culture medium, e.g., recombinant cells, and cell components.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The invention relates to the discovery of a novel approach for multiplexed detection of nucleic acids by in situ hybridization. The method combines the specificity of proximity ligation, the sensitivity of using multiple probes to target a transcript, and the high signal produced by rolling circle amplification. The engineered probes are designed to capitalize on the formation of Holliday-like junctions for optimal signal amplification. PLISH provides single molecule resolution and allows for quantitation of a virtually unlimited number of transcripts within individual cells.

In order to further an understanding of the invention, a more detailed discussion is provided below regarding molecular profiling of cells and tissue with PLISH.

A. Detecting Nucleic Acids with PLISH

The PLISH method is typically performed as follows: A tissue or cell sample is incubated with one or more pairs of probes (i.e., probe set). The two probes (referred to as right H probe and left H probe) in each probe set hybridize at adjacent sites on a target nucleic acid. The sample is then washed to remove excess unbound probes. Bridge and circle oligonucleotides, chemically or enzymatically phosphorylated at their 5′ ends, are hybridized to the bound pairs of adjacent probes. The sample is then washed to remove excess unbound bridge and circle oligonucleotides. The sample is treated with a ligase resulting in probe-templated ligation of the bridge and circle oligonucleotides to create a closed single-stranded DNA (ssDNA) circle. The sample is optionally washed to remove excess ligase. Rolling circle amplification is performed on the closed ssDNA circle, primed by the 3′ end of the right H probe. The sample is optionally washed to remove excess polymerase. Detectably labeled imager oligonucleotides are added to the sample, which hybridize to the rolling-circle amplicons, either directly or indirectly through adapter oligonucleotides. The sample is optionally washed to remove excess imager oligonucleotides. The target nucleic acids are detected by measuring a signal from the bound imager oligonucleotides. The sample can be imaged to reveal the location of the detectably labeled imager oligonucleotides complexed with the target nucleic acids.

A target nucleic acid may be any nucleic acid of interest (e.g., RNA or DNA, or a modified nucleic acid). In some embodiments, the target nucleic acid is a coding RNA (e.g., messenger RNA (mRNA)) or a non-coding RNA (e.g., transfer RNA (tRNA), ribosomal RNA (rRNA), microRNA (miRNA), mature miRNA, immature miRNA, small nuclear RNA (snRNA), or long noncoding RNA (lncRNA)). In some embodiments, the target nucleic acid is a splice variant of an RNA molecule (e.g., mRNA, pre-mRNA). The target nucleic acid may be an unspliced RNA (e.g., pre-mRNA, mRNA), a partially spliced RNA, or a fully spliced RNA.

Target nucleic acids of interest may differ in abundance within a cell population or exhibit differential expression in association with a disease or condition. The methods of the invention can be used for molecular profiling of cells to measure expression levels of nucleic acids, including without limitation RNA transcripts in individual cells.

In some embodiments, the target nucleic acid is DNA (e.g., denatured genomic, viral, or plasmid DNA). For example, the methods can be used to detect copy number variants or rare genetic variants and determine their abundances in a cell population.

The methods of the invention may be applied to cell samples comprising a single cell or a population of cells of interest and can be performed on any type of cell, including any cell from a prokaryotic, eukaryotic, or archaeon organism, including bacteria, archaea, fungi, protists, plants, and animals. Cells from tissues, organs, and biopsies, as well as recombinant cells, cells from cell lines cultured in vitro, and artificial cells (e.g., nanoparticles, liposomes, polymersomes, or microcapsules encapsulating nucleic acids) may all be used in the practice of the invention. The methods of the invention are also applicable for detecting nucleic acids in cellular fragments, cell components, or organelles comprising nucleic acids.

In some embodiments, PLISH is performed on an intact cell, naturally occurring or modified. The cell may be isolated from other cells, mixed with other cells in a culture, or within a tissue (partial or intact), or an organism. In some embodiments, the cell is lysed or permeabilized. PLISH is well suited for use with fixed cells and tissues, such as fixed cells and tissues obtained from a subject, e.g., in a clinical setting. For example, PLISH can be used on conventional formalin-fixed tissues that have been cryo- or paraffin-embedded and can be performed concurrently with immunostaining.

In some instances, the methods described herein will find use in detection, quantification, and/or mapping of RNA transcripts in a cell or tissue sample from a subject. Cell or tissue samples may be collected from any animal, including humans, livestock, pets, laboratory animals, bioproduction animals (e.g., animals used to generate a bioproduct), and the like. Mammals of interest from which such samples may be derived include but are not limited to e.g., humans, ungulates (e.g., any species or subspecies of porcine (pig), bovine (cattle), ovine (sheep) and caprine (goats), equine (horses), camelids (camels) or, generally, hooved domestic or farm animals, etc.), rodents (e.g., mice, rats, gerbils, hamsters, guinea pigs, and the like), rabbits, cats, dogs, primates, and the like.

In some instances, samples may be derived from non-human animals including but not limited to non-human mammals. Non-human mammals from which samples may be derived include but are not limited to those listed above. Non-human animals from which samples may be derived include but are not limited to those listed above and, in addition, e.g., avians (i.e., birds, such as, e.g., chicken, duck, etc.), amphibians (e.g., frogs), fish, etc.

The methods of the invention may be performed, for example, on cells, tissue, or organs of the nervous system, muscular system, respiratory system, cardiovascular system, skeletal system, reproductive system, integumentary system, lymphatic system, excretory system, endocrine system (e.g. endocrine and exocrine), or digestive system. Any type of cell can potentially be used, as described herein, including, but not limited to, epithelial cells (e.g., squamous, cuboidal, columnar, and pseudostratified epithelial cells), endothelial cells (e.g., vein, artery, and lymphatic vessel endothelial cells), and cells of connective tissue, muscles, and the nervous system. Such cells may include, but are not limited to, epidermal cells, fibroblasts, chondrocytes, skeletal muscle cells, satellite cells, heart muscle cells, smooth muscle cells, keratinocytes, basal cells, ameloblasts, exocrine secretory cells, myoepithelial cells, osteoblasts, osteoclasts, neurons (e.g., sensory neurons, motor neurons, and interneurons), glial cells (e.g., oligodendrocytes, astrocytes, ependymal cells, microglia, Schwann cells, and satellite cells), pillar cells, adipocytes, pericytes, stellate cells, pneumocytes, blood and immune system cells (e.g., erythrocytes, monocytes, dendritic cells, macrophages, neutrophils, eosinophils, mast cells, T cells, B cells, natural killer cells), hormone-secreting cells, germ cells, interstitial cells, lens cells, photoreceptor cells, taste receptor cells, and olfactory cells; as well as cells and/or tissue from the kidney, liver, pancreas, stomach, spleen, gall bladder, intestines, bladder, lungs, prostate, breasts, urogenital tract, pituitary cells, oral cavity, esophagus, skin, hair, nail, thyroid, parathyroid, adrenal gland, eyes, nose, or brain.

At least one probe set is provided for each target nucleic acid to be detected, wherein each probe set comprises: i) a first probe comprising a 5′ overhang region and a region that hybridizes to the target nucleic acid at a first target site; ii) a second probe comprising a 3′ overhang region and a region that hybridizes to the target nucleic acid at a second target site.

A target site is a complementary region of the target nucleic acid to which a probe binds. A pair of probes in a probe set bind to a pair of different target sites that are sufficiently close together to allow simultaneous hybridization to a bridge oligonucleotide. The probes will usually hybridize to two adjacent regions (i.e., target sites) on the target nucleic acid, which may be separated by 0 up to 150 nucleotides, including any number of nucleotides in this range such as 0, 1, 2, 3, 4, 5, 6, 7, 8, 9,10, 15, 20, 25, 30, 35, 40, 45, 50, 55, 60, 65, 70, 75, 80, 85, 90, 100, 110, 120, 130, 140, or 150 nucleotides. A zero nucleotide gap means that the two regions or target sites directly abut one another. In other words, the two regions bound by a pair of probes may be contiguous, i.e. there is no gap between the two target sites. Alternatively, the two regions hybridized by the probe oligonucleotides may be separated by 1 to about 150 nucleotides. Target sites are typically present on the same strand of the target nucleic acid in the same orientation. Target sites are usually selected to provide a unique binding site not present in other nucleic acids in the sample. Each target site is generally from about 18 to about 30 nucleotides in length, or any length within this range such as 18, 19, 20, 21, 22, 23, 24, 25, 26, 27, 28, 29, or 30 nucleotides in length.

In some embodiments, the probes in a probe set have a similar melting temperature for binding to their cognate target sites. For example, the Tₘ may range from about 45° C. to about 65° C., including any Tₘ within this range such as 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C.

A bridge oligonucleotide hybridizes to a pair of probes to form a complex on a target nucleic acid. The bridge oligonucleotide comprises i) a first portion that hybridizes to a complementary region in the 5′ overhang region of one probe of the pair, and ii) a second portion that hybridizes to a complementary region in the 3′ overhang region of the second probe of the pair. The first probe and the second probe, when bound to a target nucleic acid, are in sufficient proximity to each other to simultaneously hybridize to the bridge oligonucleotide to allow formation of the complex with the bridge oligonucleotide on the target nucleic acid. A signal is only generated when two probes hybridize sufficiently close to each other on a target nucleic acid to allow hybridization of the circle oligonucleotide in this manner.

A circle oligonucleotide comprises a first portion that hybridizes to a complementary region at the 5′ end of the 5′ overhang region of the first probe of a probe set, and a second portion that hybridizes to a complementary region at the 3′ end of the 3′ overhang region of the second probe of the probe set. Circular DNA forms where any two probes of a probe set bind sufficiently close to each other on one of the target nucleic acids to allow ligation of a bridge oligonucleotide and circle oligonucleotide that are hybridized to the two probes to generate a closed circle.

Rolling circle amplification (RCA) is performed with each circular DNA molecule formed serving as a template to produce a concatemer comprising multiple copies of the circular DNA nucleotide sequence. RCA is an isothermal nucleic acid amplification technique that uses a polymerase to extend a primer annealed to a circular template to produce a long ssDNA concatemer that contains tens to hundreds of tandem repeats of a sequence complementary to the circular template. A strand-displacing polymerase, such as Phi29, Bst, or Vent exo-DNA polymerase can be used for rolling circle amplification. For a description of RCA, see, e.g., Ali et al. (2014) Chemical Society Reviews 43 (10):3324-3341, Demidov (2002) Expert Rev. Mol. Diagn. 2(6):542-548; herein incorporated by reference.

The length of the oligonucleotide reagents (e.g., probes, circle oligonucleotides, bridge oligonucleotides, and imager oligonucleotides) will vary and may be 10 or more nucleotides and range from 10 to 100 or more nucleotides, including e.g., 10 to 100 nucleotides, 20 to 90 nucleotides, 30 to 80 nucleotides, 40 to 60 nucleotides, 10 to 50 nucleotides, 12 to 50 nucleotides, 14 to 50 nucleotides, 16 to 50 nucleotides, 18 to 50 nucleotides, 20 to 50 nucleotides, 22 to 50 nucleotides, 24 to 50 nucleotides, 26 to 50 nucleotides, 28 to 50 nucleotides, 30 to 50 nucleotides, 10 to 40 nucleotides, 12 to 40 nucleotides, 14 to 40 nucleotides, 16 to 40 nucleotides, 18 to 40 nucleotides, 20 to 40 nucleotides, 22 to 40 nucleotides, 24 to 40 nucleotides, 26 to 40 nucleotides, 28 to 40 nucleotides, 30 to 40 nucleotides, 10 to 30 nucleotides, 12 to 30 nucleotides, 14 to 30 nucleotides, 16 to 30 nucleotides, 18 to 30 nucleotides, 20 to 30 nucleotides, 12 or more nucleotides, 13 or more nucleotides, 14 or more nucleotides, 15 or more nucleotides, 16 or more nucleotides, 17 or more nucleotides, 18 or more nucleotides, 19 or more nucleotides, 20 or more nucleotides, 30 or more nucleotides, 40 or more nucleotides, 50 or more nucleotides, 60 or more nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 35 nucleotides, 40 nucleotides, 45 nucleotides, 50 nucleotides, 55 nucleotides, 60 nucleotides etc. Exemplary oligonucleotide sequences for probes, circle oligonucleotides, bridge oligonucleotides, and imager oligonucleotides are shown in Example 1 and SEQ ID NOS:1-464 of the Sequence Listing.

In some instances, the oligonucleotides of the subject disclosure may include one or more nucleoside analogs. For example, in some instances, imager oligonucleotides of the instant disclosure may include one or more deoxyribouracil (i.e., deoxyribose uracil, -deoxyuridine, etc.) nucleosides/nucleotides. In certain instances, an oligonucleotide may include 2 or more nucleoside analogs including but not limited to e.g., 3 or more, 4 or more, 5 or more, 6 or more, etc. In some instances, the number of nucleoside analogs as a percentage of the total bases of an oligonucleotide is 1% or more, including but not limited to e.g., 2% or more, 3% or more, 4% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, etc.

Probes, circle oligonucleotides, bridge oligonucleotides, and imager oligonucleotides for use in the assays described herein are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly(A) or poly(C), or other non-complementary nucleotide extensions may be incorporated into oligonucleotides using these same methods. Hexaethylene oxide extensions may be coupled to the oligonucleotides by methods known in the art. Cload et al., J. Am. Chem. Soc. (1991) 113:6324-6326; U.S. Pat. No. 4,914,210 to Levenson et al.; Durand et al., Nucleic Acids Res. (1990) 18:6353-6359; and Horn et al., Tet. Lett. (1986) 27:4705-4708.

B. Multiplexing

The methods described herein can be readily used to screen a sample for the presence of target nucleic acids. The methods are suitable for detection of a single target nucleic acid as well as multiplex analyses in which two or more different target nucleic acids are detected in a sample. In some instances, multiple nucleic acids (e.g., RNA transcripts) may be screened in a single sample, and the presence or quantities of each target nucleic acid may be assessed. The detection methods described herein may be utilized in parallel for the detection and measurement of large numbers of target nucleic acids in a cell or tissue sample. The methods of the invention are capable of highly sensitive and highly multiplexed assessment of many different target nucleic acids in a single sample.

In some embodiments, a plurality of different target nucleic acids are detected in a sample, such as up to 2, up to 3, up to 4, up to 5, up to 6, up to 7, up to 8, up to 9, up to 10, up to 12, up to 15, up to 18, up to 20, up to 25, up to 30, up to 40, up to 50, up to 60, up to 70, up to 80, up to 90, up to 100, up to 500, up to 1000, or more distinct target nucleic acids.

A multiplexed assay may make use of various different probes, circle oligonucleotides, bridge oligonucleotides, and uniquely labeled imager oligonucleotides for detection of particular target nucleic acids. For multiplex assays, the number of different probe sets, circle oligonucleotides, bridge oligonucleotides, and imager oligonucleotides that may be employed typically ranges from about 2 to about 20 or higher, e.g., up to 100 or higher, 1000 or higher, etc., including but not limited to e.g., 2 to 50, 2 to 100, 10 to 100, 50 to 100, 50 to 200, 50 to 300, 50 to 400, 50 to 500, etc.

Multiplexed assays are generally performed using a plurality of probe sets. The number of probe sets will vary depending on the number and/or type of target nucleic acids to be screened. Accordingly, in some instances, probe set libraries may be used for screening large numbers of target nucleic acids. Libraries may be categorized by the type of RNA transcripts targeted by probes contained in the library, including e.g., libraries which contain various probes for detection of mRNAs in particular cell types, tissues, or organs, or associated with particular disease states, developmental stages, or physiological conditions.

The number of different probes sets will vary and may range from 10 or less to 1000 or more, including but not limited to e.g., 10 to 1000, 20 to 1000, 30 to 1000, 40 to 1000, 50 to 1000, 60 to 1000, 70 to 1000, 80 to 1000, 90 to 1000, 100 to 1000, 100 to 900, 100 to 800, 100 to 700, 100 to 600, 100 to 500, 100 to 400, 100 to 300, 100 to 200, 10 to 900, 10 to 800, 10 to 700, 10 to 600, 10 to 500, 10 to 400, 10 to 300, 10 to 200, 10 to 100, 20 to 100, 30 to 100, 40 to 100, 50 to 100, 60 to 100, 70 to 100, 80 to 100, 90 to 100, 5, 10, 15, 20, 25, 30, 35, 40, 45, 50, 60, 70, 80, 90, 100, 250, 500, 1000, etc. The different probes of a library may be physically separated, e.g., in separate containers or separate wells of a multi-well plate, or may not be physically separated, i.e., may be pooled, in a single solution, in a single container, etc.

In some instances, a library of probe sets may include a corresponding library of circle oligonucleotides, bridge oligonucleotides, or imager oligonucleotides for multiplexed detection of the target nucleic acids. Libraries of the present disclosure may also include one or more additional reagents for performing all or part of a method as described herein, including e.g., additional reagents for ligation, rolling circle amplification, detection, etc. In some instances, additional reagents may be included in a pooled library. For example, in some instances, reagents for ligation (e.g., a ligase) or rolling circle amplification (polymerase and deoxyribonucleotides) may be included within a pooled library of probe sets. In some instances, additional reagents may be included in the individual wells of a multi-well plate. For example, in some instances, reagents for ligation or rolling circle amplification (e.g., a polymerase, dNTPs, etc.) may be included within the wells of a multi-well plate probe set library. Appropriate buffers, salts, etc. may or may not be included in the libraries as described. In some instances, libraries and/or components thereof, e.g., a probe set library, may be provided in a lyophilized form and may be rehydrated upon use.

C. Detection

The presence of target nucleic acids is determined by using detectably labeled imager oligonucleotides that bind to sites in the circular DNA sequence that is amplified by rolling circle amplification. Imager oligonucleotides may be detectably labeled with any molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. Representative examples of detectable labels, which may be used in the practice of the invention, include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647,and Alexa Fluor 784, a cyanine dye such as Cy 3, Cy3.5, Cy5, Cy5.5, and Cy7, fluorescein, 2′,4′,5′,7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neo®, G418′) dihydrofolate reductase (DHFR), hygromycin-B-phosphotransferase (HPH), thymidine kinase (TK), β-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. Detectable labels also include chemiluminescent labels such as luminol, isoluminol, acridinium esters, and peroxyoxalate and bioluminescent proteins such as firefly luciferase, bacterial luciferase, Renilla luciferase, and aequorin. Detectable labels also include isotopic labels, including radioactive and non-radioactive isotopes, such as, 3H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Ln, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr. Detectable labels also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.), near infrared (NIR) probes, and nanoshells. Detectable labels also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

The label may be a directly detectable label, which can be directly detected without the use of additional reagents, or an indirectly detectable label, which is detectable by employing one or more additional reagents (e.g., where the label is a member of a signal producing system made up of two or more components). In some embodiments, the imager oligonucleotides comprise directly detectable labels such as, but not limited to, fluorescent labels, radioisotopic labels, chemiluminescent labels, chelated metals, and the like.

In some embodiments, the label is a fluorescent label, wherein detection of a target nucleic acid involves detection of a fluorescent signal from bound imager oligonucleotides. A concatemer comprising a repeating circular DNA sequence is produced by rolling circle amplification, and the amplification product is detected by hybridization of one or more fluorescently labeled imager oligonucleotides to the amplification product. Any convenient means for detecting fluorescence may be used for detecting the bound imager oligonucleotides, including but not limited to, e.g., fluorescence microscopy, flow cytometry, imaging flow cytometry, etc.

For multiplex assays, each RNA species can be detectably labeled in a unique color by using imager oligonucleotides with spectrally-distinct fluorophores. Fluorescence micrographs can be interpreted by direct visual inspection. Typically, up to five distinct channels can be simultaneously detected and imaged by conventional fluorescence microscopy, as well as allowing a determination of RNA abundance.

Multiple cycles of fluorescence imaging may be performed to allow detection of larger numbers of transcripts. Subsets of the target nucleic acids may be imaged sequentially. For example, a sample may be contacted with a subset of the imager oligonucleotides designed for detection of specific target nucleic acids, followed by performing a cycle of fluorescence imaging. Before performing another round of fluorescence imaging, the imager oligonucleotides are removed from the sample, for example, by using a wash step. Then, additional imager oligonucleotides are added to the sample to detect additional target nucleic acids.

Highly multiplexed measurement of different RNA species may require a large number of iterated data collection cycles. Ideally, the cycles should be fast, and removal of the bound imager oligonucleotides between cycles should not cause any mechanical or chemical damage to the sample. Short imager oligonucleotides (e.g., up to 11 nucleotides in length), which equilibrate rapidly on and off of the RCA amplicons, can be removed with a simple buffer exchange (see Example 1). Alternatively, uracil-containing imager oligonucleotides can be used, which can be readily removed by a brief enzymatic digestion (e.g., see Example 1 for a description of removal of uracil-containing imager oligonucleotides with uracil-specific excision reagent (USER) enzyme).

In certain embodiments, RNA species are imaged in sets of 5, with differently colored fluorophores associated with different targets (most fluorescence microscopes can only accommodate 5 color channels). In order to overcome the limit of 5 color channels on a typical fluorescence microscope, iterative rounds of staining, imaging and erasing can be used to colocalize large numbers of distinct RNA species in sequential images.

D. Applications

The methods and compositions described herein have particular utility in the detection, quantification, and/or mapping of target nucleic acids present in a sample. Such detection may find various applications in a variety of technological fields including but not limited to e.g., basic scientific research (e.g., biomedical research, biochemistry research, immunological research, molecular biology research, microbiological research, cellular biology research, genetics, and the like), medical and/or pharmaceutical research (e.g., drug discovery research, drug design research, drug development research, pharmacology, toxicology, medicinal chemistry, preclinical research, clinical research, personalized medicine, and the like), medicine, epidemiology, public health, biotechnology, veterinary science, veterinary medicine, agriculture, material science, molecular detection, molecular diagnostics, and the like.

Multiplexed assays can be used in molecular profiling to identify distinct cell-types and cell populations. The methods of the invention can be used to map all or some of the molecularly distinct cell types that make up a complex tissue based on their expression of target nucleic acids. Multiplexed assays can be used, for example, in molecular profiling to identify distinct cell populations within a tissue to determine the organization of cells in various systems including solid tumors and developing organs.

In particular, the methods of the invention should have many applications, for example, in the discovery and localization of novel cell types, the mapping of signaling centers, analysis of development, or molecular profiling of cell-types associated with disease. The methods of the invention can be used in analysis of formalin-fixed and paraffin-embedded samples, cryo-preserved samples and legacy tissue bank samples. In particular, the methods are applicable to clinical pathology labs. Additionally, the methods can be used in medical diagnostics based on multiplexed expression profiling in primary patient samples, with no prior purification or isolation of cells. Examples include: (a) direct liquid biopsy, such as for detection of circulating cancer cells or fetal cells by profiling patient blood products on a microscope slide, (b) quality control of patient stem cells monitoring the gene expression of stem cells that are being differentiated ex vivo for therapeutic purposes, and (c) discovery and use of context-dependent biomarkers, i.e., biomarkers that provide a definitive diagnosis when observed in a specific tissue context.

E. Automated Image Analysis and Cell Classification

In some embodiments, image analysis and identification of cell types in a tissue based on the detected target nucleic acids present is automated by use of an algorithm or classifier. Automated analysis will be particularly useful for multiplex assays involving detection of large numbers of RNA transcripts. Cell types can be identified and classified using techniques known in the art. For example, a machine learning algorithm or clustering algorithm may be used.

The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include a t-distributed stochastic neighbor embedding algorithm, artificial neural network, data clustering, expectation-maximization algorithm, self-organizing map, radial basis function network, vector quantization, generative topographic map, information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

F. Kits

The above-described assay reagents, including probes, circle oligonucleotides, bridge oligonucleotides, and imager oligonucleotides, and optionally reagents for performing ligation and rolling circle amplification can be provided in kits, with suitable instructions and other necessary reagents, in order to conduct the assays for detecting target nucleic acids (e.g., DNA or RNA transcripts) as described above. The kit will normally contain in separate containers the probes, circle oligonucleotides, bridge oligonucleotides, and imager oligonucleotides, and other reagents that the assay format requires. Instructions (e.g., written, CD-ROM, DVD, Blu-ray, flash drive, digital download, etc.) for carrying out the assays usually will be included in the kit. The kit can also contain, depending on the particular assay used, other packaged reagents and materials (i.e., wash buffers, and the like). Assays for detecting nucleic acids, as described herein, can be conducted using these kits.

In certain embodiments, the kit comprises one or more oligonucleotide reagents (e.g., probe, circle, bridge, and imager oligonucleotides) comprising a nucleotide sequence selected from the group consisting of SEQ ID NOS:1-464, or sequences displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Automated Cell-Type Classification in Intact Tissues by Single-Cell Molecular Profiling

Introduction

Here, we report an in situ hybridization technique with performance characteristics that enable rapid and scalable single-cell expression profiling in tissue. Our approach is a simplified variant of the padlock/RCA technique which replaces padlock probes with RNA-templated proximity ligation (Soderberg et al. (2006) Nature Methods 3:995-1000; Frei et al. (2016) Nature Methods 13:269-275) at Holliday junctions (Labib et al. (2013) Analytical Chemistry 85:9422-9427); hence, we term it proximity ligation in situ hybridization (PLISH).

As demonstrated below, PLISH generates data of exceptionally high signal-to-noise. Multiplexed hybridization and signal amplification of all target RNA species is carried out in a single parallel reaction, and the RNAs are then localized with rapid label-image-erase cycles. PLISH exhibits high detection efficiency because it probes multiple sites in each target RNA, and high specificity because of the proximity ligation mechanism. PLISH utilizes only commodity reagents, so it can be scaled up inexpensively to cover many genes. It works well on conventional formalin-fixed tissues that have been cryo- or paraffin-embedded, and can be performed concurrently with immunostaining, making it extremely versatile.

Using the murine lung as a characterized model tissue, we show that multiplexed PLISH can rediscover and spatially map the distinct cell types of a tissue in an automated and unsupervised fashion. An unexpected discovery from this experiment is that murine Club cells separate into two populations that differ molecularly and segregate anatomically. PLISH constitutes a novel, single cell spatial-profiling technology that combines high performance, versatility and low cost. Because of its technical simplicity, it will be accessible to a broad scientific community.

Results

Proximity Ligation In Situ Hybridization (PLISH)

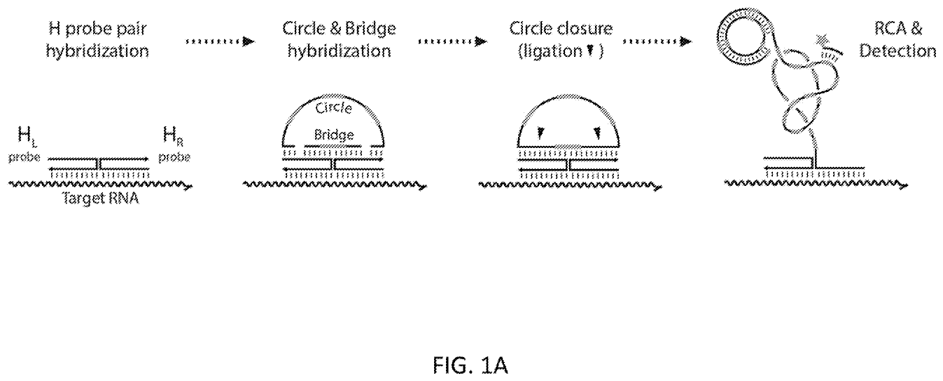

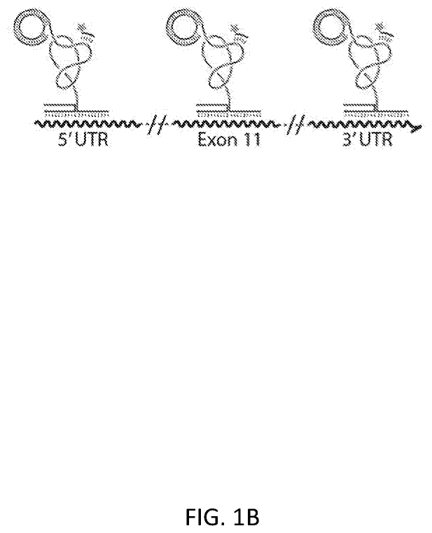

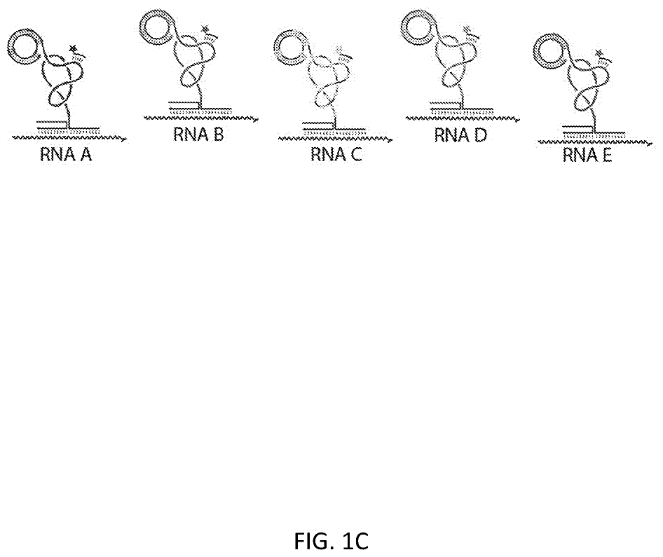

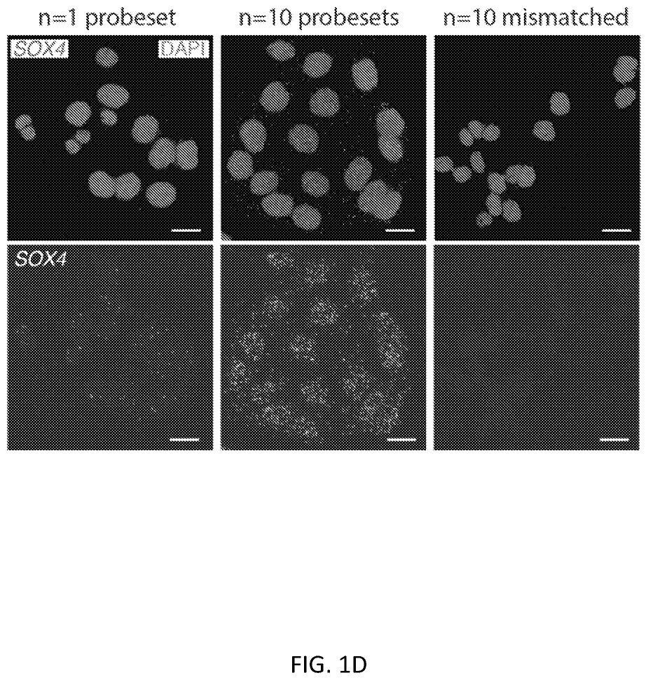

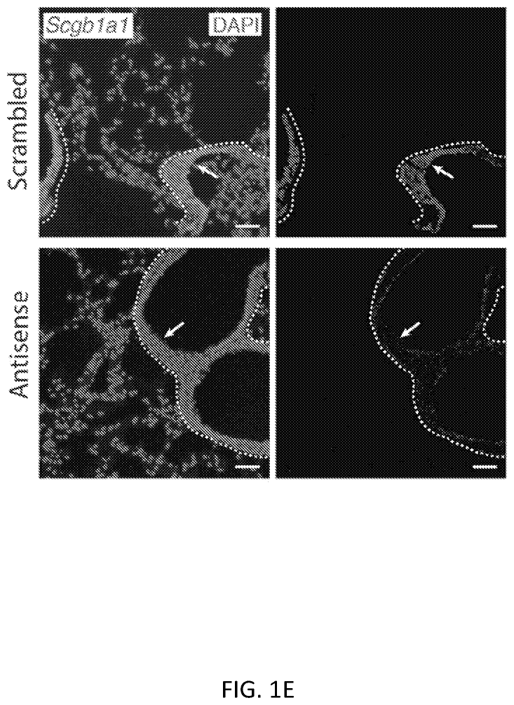

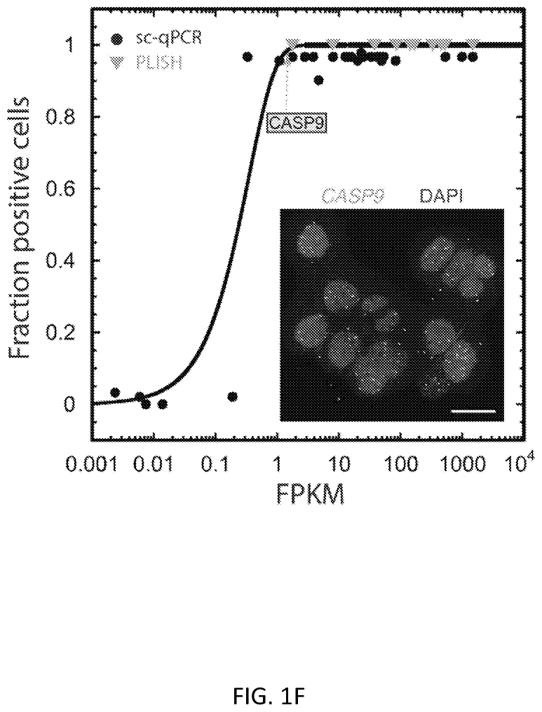

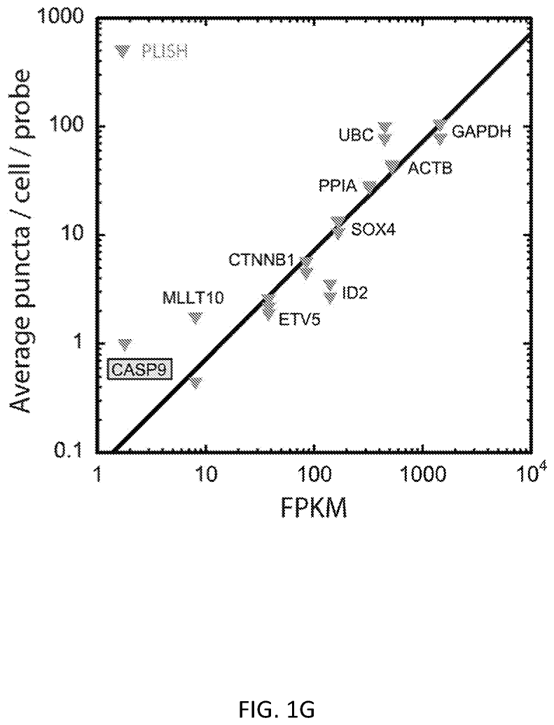

Proximity ligation at Holliday junctions offers a simple mechanism for the amplified detection of RNA (Labib et al., supra). First, a transcript is targeted with a pair of oligonucleotide ‘H’ probes designed to hybridize at adjacent positions along its sequence (FIG. 1A). The left H probe includes a single-stranded 5′ overhang while the right probe includes a 3′ overhang. Importantly, target RNAs can be tiled with H probe pairs at multiple sites, which is critical for efficient detection of low abundance transcripts (FIG. 1B). The overhangs are then hybridized to ‘bridge’ and linear ‘circle’ oligonucleotides with embedded barcode sequences to form a Holliday junction structure, after which ligation at the nick sites creates a closed circle. Finally, the 3′ end of the right H probe primes rolling-circle replication, which generates a long single-stranded amplicon of tandem repeats. Addition of fluorescently-labeled ‘imager’ oligonucleotides complementary to the barcodes generates an extremely bright punctum at the site of each labeled transcript. Because each barcode sequence is unique, the puncta derived from different target RNAs can be labeled with different colors (FIG. 1C).

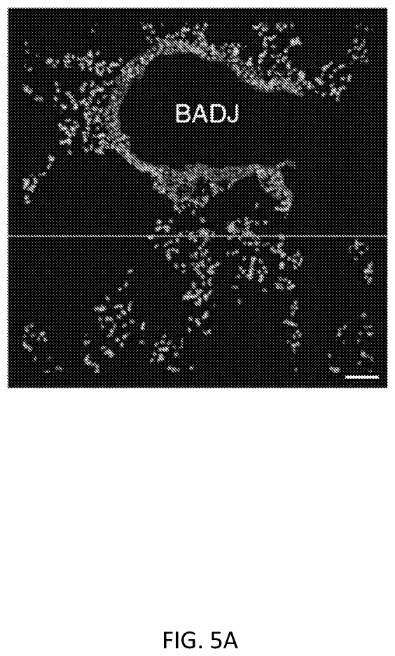

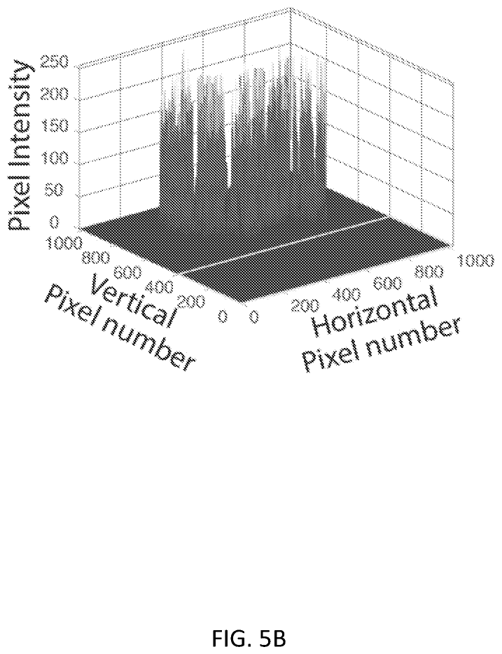

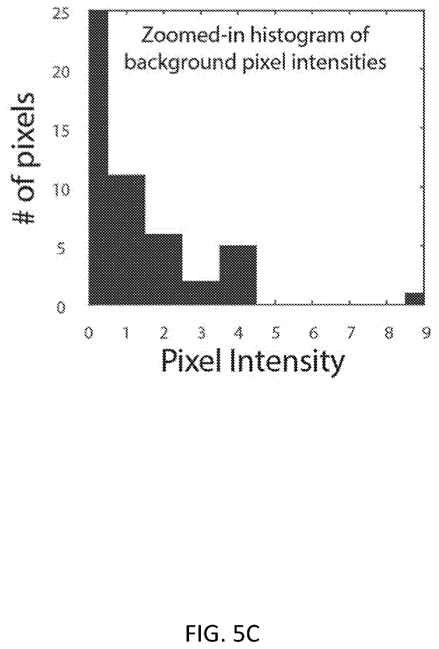

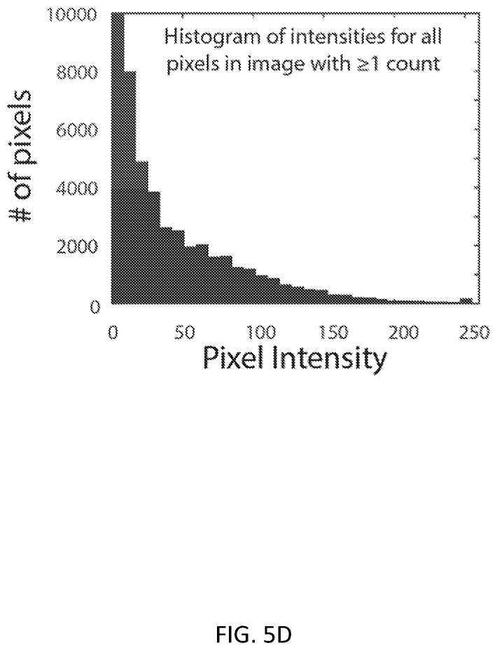

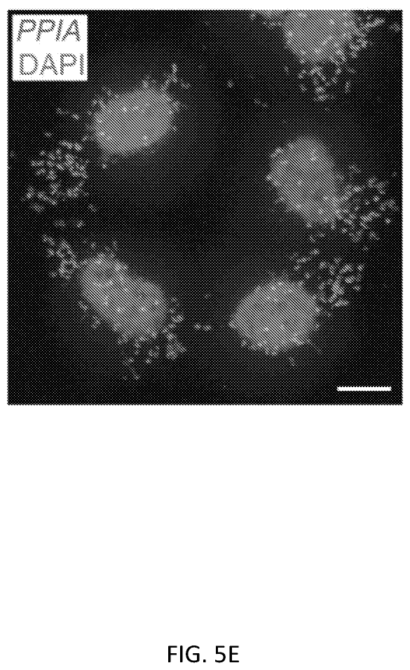

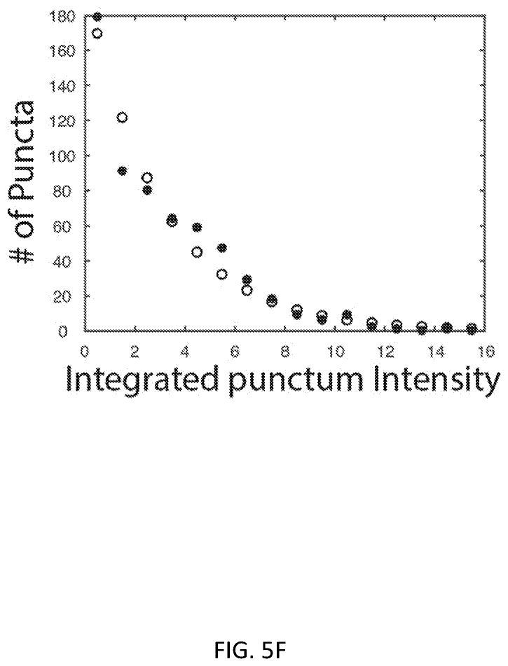

To implement PLISH, we adapted protocols for antibody-based proximity ligation (Soderberg et al., supra). The technique utilizes conventional oligonucleotides, two commercially available enzymes, and procedures familiar to molecular biologists. The ligase and polymerase enzymes are less than half the size of an immunoglobulin G, and they diffuse at least as rapidly as the 60 mer DNA hairpins used for HCR amplification (Choi et al. (2014) ACS Nano 8:4284-4294; Joubert et al. (2003) Journal of Biological Chemistry 278:25341-25347; Lapham et al. (1997) Journal of Biomolecular NMR 10:255-262; Modrich et al. (1973) The Journal of Biological Chemistry 248:7495-7501). Our initial studies produced bright puncta that were absent if any of the oligonucleotide or enzyme reagents was withheld. The signal from the individual RCA amplicons exceeded cellular and tissue fluorescence background by more than 30-fold, rendering autofluorescence inconsequential (FIGS. 5A-5B and Jarvius et al. (2006) Nature Methods 3:725-727; Blab et al. (2004) Analytical Chemistry 76:495-498). Histograms of puncta intensities fit to a negative binomial distribution, as expected for a DNA replication process that terminates stochastically and irreversibly (FIGS. 5C-5F). The coefficients of variation for the puncta intensity distributions were typically between one and two.

Highly Specific and Sensitive Detection of RNA Transcripts

The requirement for coincident hybridization of two probes at adjacent sites in an RNA transcript should make PLISH highly specific. To evaluate this, we performed several experiments. First, we used PLISH to detect the transcription factor SRY-box 4 (SOX4) in cultured HCT116 cells. A pool of ten H probe sets exhibited much higher RNA detection efficiency than a single H probe set, as expected (FIG. 1D). However, when the RNA-recognition sequence of either the left or right H probe in each set was scrambled, there were no detectable puncta. Thus, both H probes had to be correctly targeted to generate a signal.

Second, we tested the sequence-specificity of the PLISH signal in tissue by pre-incubating samples with antisense ‘blocking’ oligonucleotides complementary to the target RNA at the H probe hybridization sites. For these experiments, we stained mouse lung sections for secretoglobin 1a1 (Scgb1a1), a marker of airway Club cells. Antisense oligonucleotides drastically attenuated the number of PLISH puncta, whereas scrambled blocking oligonucleotides of the same length had no apparent effect (FIG. 1E).

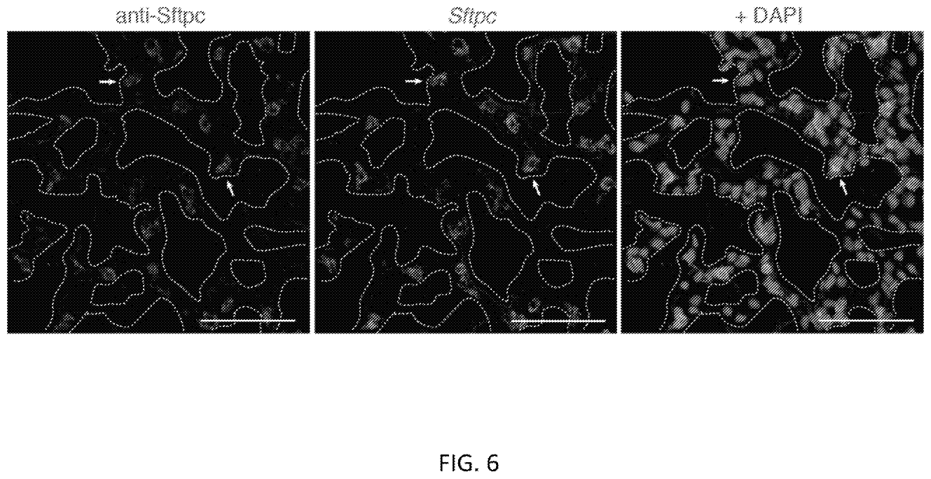

Third, we analyzed murine lung sections for the co-localization of the mRNA transcript and protein product of surfactant protein C (Sftpc), which is expressed in alveolar epithelial type II (AT2) cells. Of the cells that were positive for PLISH signal, 98.5% were also positive for antibody staining (n=184, FIG. 6). This level of specificity is excellent relative to HCR-amplified smISH, where off-target binding of hybridization probes can account for a quarter of the observed puncta (Shah et al. (2016) Neuron 92:342-357).

To quantify the sensitivity and accuracy of RNA detection, we benchmarked PLISH measurements against a reference-standard dataset of single-cell, quantitative reverse transcription polymerase chain reaction (qPCR) and RNA-seq measurements on HCT116 cells (Wu et al. (2014) Nature Methods 11:41-46). For genes with fragment-per-kilobase-per-million-read (FPKM) values greater than one, the single-cell qPCR technique detected mRNA in >90% of the cells (FIG. 1F). However, the fraction of transcript-positive cells dropped quickly between FPKM values of 1 and 0.1. A fit of the qPCR data to a Poisson sampling model suggested that an FPKM value of one corresponded to 2.5 copies per cell (see also Marinov et al. (2014) Genome Research 24:496-510; Battich et al. (2013) Nature Methods 10:1127-1133). The PLISH technique detected RNA transcripts with a sensitivity comparable to single-cell qPCR. For example, Caspase-9 (CASP9) has an FPKM value of 2, and it was observed in 100% of the cells by PLISH. We detected an average of 8 puncta per cell, which is consistent with the prediction of 5 copies per cell from the fit to the qPCR data (FIG. 1F, inset). For a set of ten genes covering the full spectrum of expression levels in HCT116 cells, the number of PLISH puncta per cell correlated with bulk FPKM values (FIG. 1G).

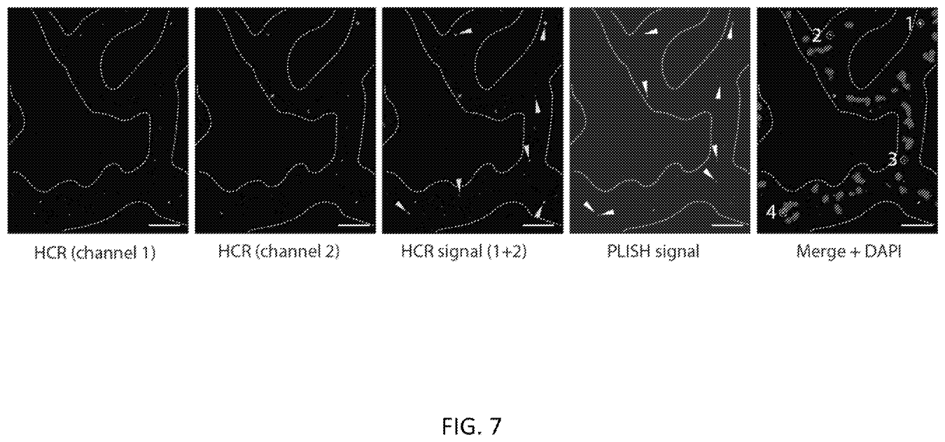

To quantify RNA-detection efficiency in tissue, we marked a set of axin 2 (Axin2) transcripts in mouse lung sections using an HCR-amplified smISH procedure (Choi et al. (2014) ACS Nano 8:4284-4294) and then determined the fraction of the marked transcripts that could be identified by PLISH. We chose the Axin2 gene because of its low expression level in the lung. HCR detected a sparse population of cells with one to two puncta each (the HCR detection efficiency was low because we used a single HCR probe rather than 24). PLISH puncta generated with a pool of four H probe pairs co-localized with 32% of the HCR puncta (FIG. 7). Thus, the four PLISH probe sets detected Axin2 transcripts with a composite efficiency of 32% and an average per-site efficiency of 9%. This probe efficiency matches or exceeds that of other smISH techniques. The PLISH detection efficiency can be tuned on a per gene basis by altering the number of H probe pairs. Decreasing the number of probe sets pro-rates the number of puncta from highly-expressed genes, while increasing the number of probe sets can facilitate sensitive detection of very low-abundance transcripts.

Visualization of Molecular and Histological Features in Tissue

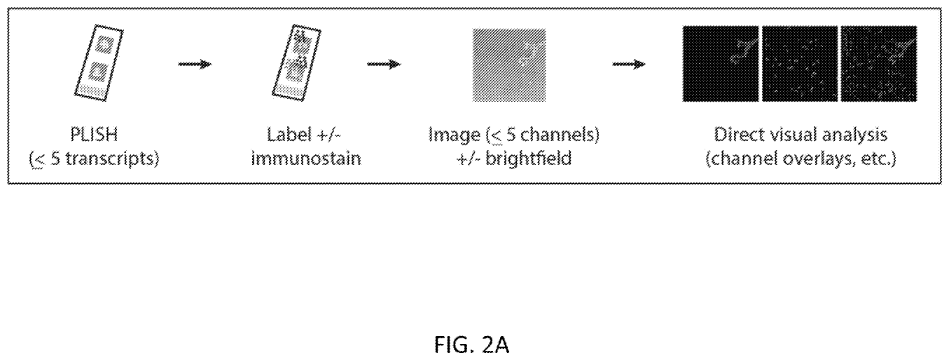

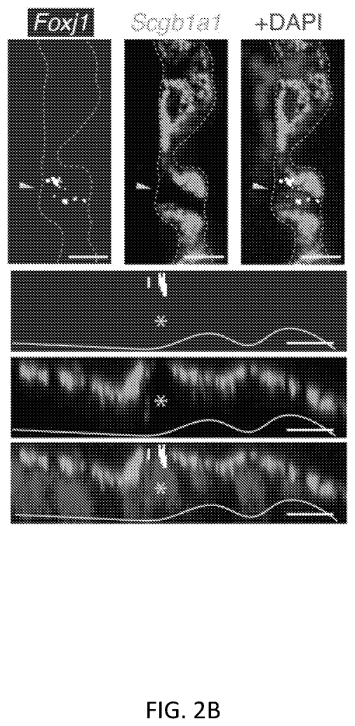

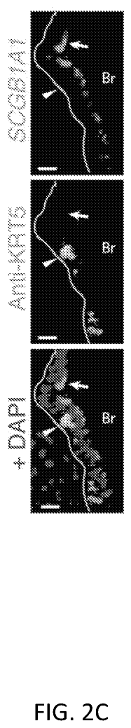

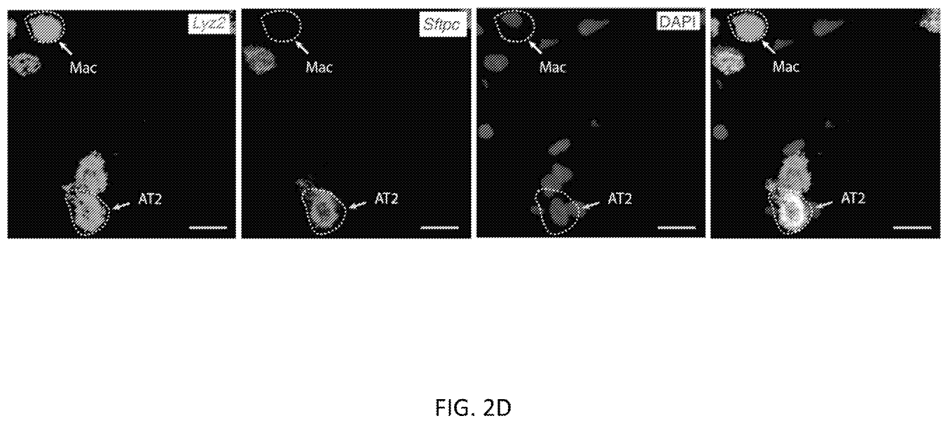

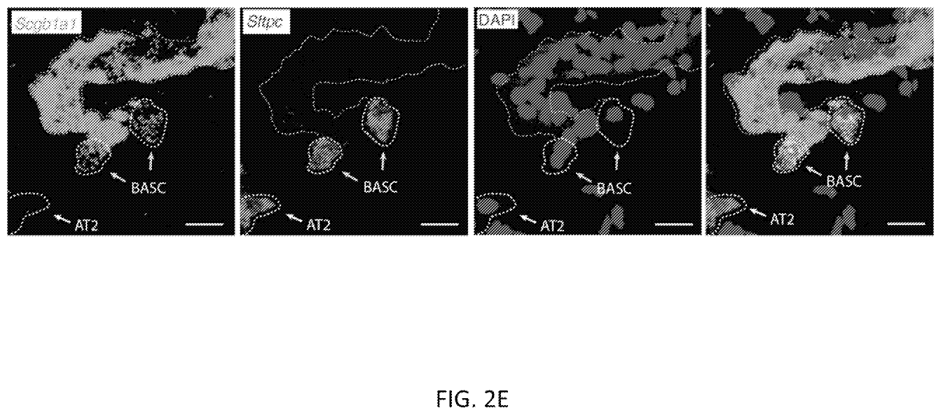

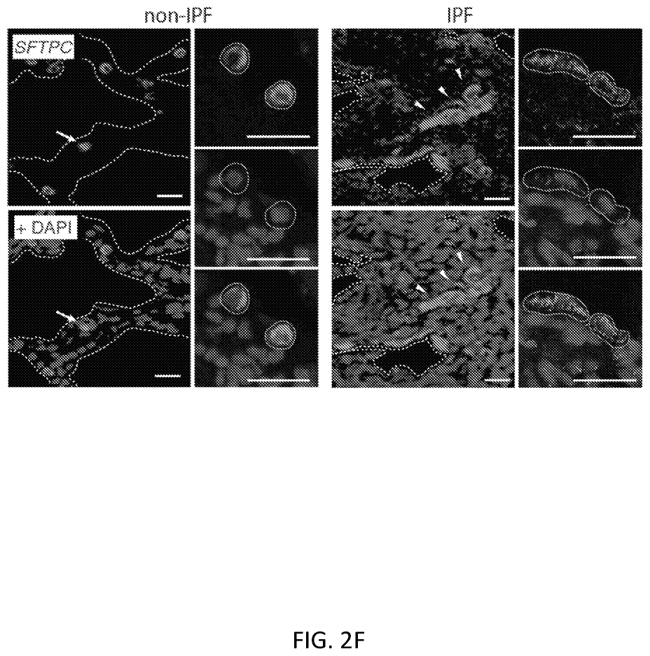

We next characterized the performance of PLISH for low-plex RNA localization in tissues. This experimental format uses a disposable hybridization chamber that is sealed to a coverslip or slide surrounding a tissue section (FIG. 2A). PLISH detection of up to 5 RNA species is accomplished by stepwise application of reagents through the inlet and outlet ports of the chamber. The puncta from each RNA species are then labeled in a unique color by hybridization to ‘imager’ oligonucleotides with spectrally-distinct fluorophores. After imaging, the fluorescence micrographs are interpreted by direct visual inspection. We aimed to test whether PLISH provides single-molecule and single-cell resolution in tissues, whether it robustly detects low-abundance RNA species, whether the spatial distribution of RNA is consistent with prior knowledge, whether PLISH is compatible with simultaneous immunostaining, and whether it is compatible with formalin-fixed, paraffin-embedded (FFPE) samples.

First, we analyzed murine lung sections for RNA expression of the ciliated-cell marker Forkhead box J1 (Foxj1), and the Club-cell marker Scgb1a1. Foxj1 is a low-abundance transcript with an FPKM value of 10 in ciliated cells, as measured by scRNA-seq (Treutlein et al. (2014) Nature 509:371-375). We observed single cells with multiple discrete Foxj1 puncta in the terminal bronchiolar epithelium, surrounded by numerous strongly Scgb1a1 positive cells (FIG. 2B). These data establish PLISH's single-molecule and single-cell resolution in tissues, and its ability to detect low-abundance transcripts.

Second, we analyzed human lung FFPE sections for RNA expression of SCGB1A1, and for protein expression of the basal cell marker, Keratin 5 (KRT5). To do this, we appended two antibody incubation steps to the standard PLISH protocol. Strongly SCGB1A1 positive cells were localized to the lumen of the airways, overlying KRT5 positive cells (FIG. 2C), matching the known anatomical distribution of Club and basal cells, respectively. These data establish PLISH's compatibility with simultaneous immunostaining, and with FFPE samples.

Third, we analyzed murine lung sections for RNA expression of three genes: the AT2 cell marker Sftpc, the macrophage-enriched marker Lysozyme 2 (Lyz2), and Scgb1a1. Overlays of the three channels provided a striking visual depiction of the different cell types. Macrophages were bright in the Lyz2 channel, but absent in the other channels (FIG. 2D). AT2 cells were bright in the Sftpc channel, moderately bright in the Lyz2 channel and absent in the Scgb1a1 channel (FIG. 2D, white cells in the overlay). Club cells were very bright in the Scgb1a1 channel, but otherwise absent (FIG. 2E). Finally, putative bronchioalveolar stem cells (Kim et al. (2005) Cell 121:823-835) (BASCs) were bright in the Sftpc channel with a weak punctate signal in the Scgb1a1 channel (FIG. 2E). Thus, raw PLISH data can be interpreted without any computational processing, made possible by PLISH's exceptional signal-to-noise in tissues.

We also evaluated how PLISH performs in primary samples of diseased human tissue, to assess whether it will be useful for molecular analysis of the many human diseases that cannot be accurately modeled in animals. One example is idiopathic pulmonary fibrosis (IPF), a fatal lung disease of unknown pathogenesis (Travis et al. (2013) American Journal of Respiratory and Critical Care Medicine 188:733-748). The diagnosis of IPF is based on the presence of specific histological features, including clusters of spindle-shaped fibroblasts, stereotyped ‘honeycomb’ cysts, and epithelial cell hyperplasia. In this regard, single-cell profiling approaches that operate on dissociated tissue (Xu et al. (2016) JCI Insight 1:e90558) are intrinsically limited because they cannot correlate molecular data with cytologic and spatial features. As a preliminary test, we used PLISH to analyze RNA expression of the AT2 cell marker SFTPC in resected lung tissue from control and IPF patients. In contrast to the uniformly cuboidal SFTPC-expressing AT2 cells distributed throughout alveoli of non-IPF lungs (FIG. 2F), we observed clusters of SFTPC^{Hi} cells of heterogeneous size and varying degrees of flattening lining the airspace lumen of IPF lungs. Surprisingly, many cells that did not appear to be epithelial (i.e., they were not lining an airway lumen) expressed SFTPC at low levels. Based on this pilot experiment, PLISH should be a suitable tool for building atlases of RNA expression in human disease. The PLISH data can be overlaid with monoclonal antibody staining patterns that are the mainstay of pathologic diagnosis and classification.

Multiplexed and Iterative PLISH in Tissues

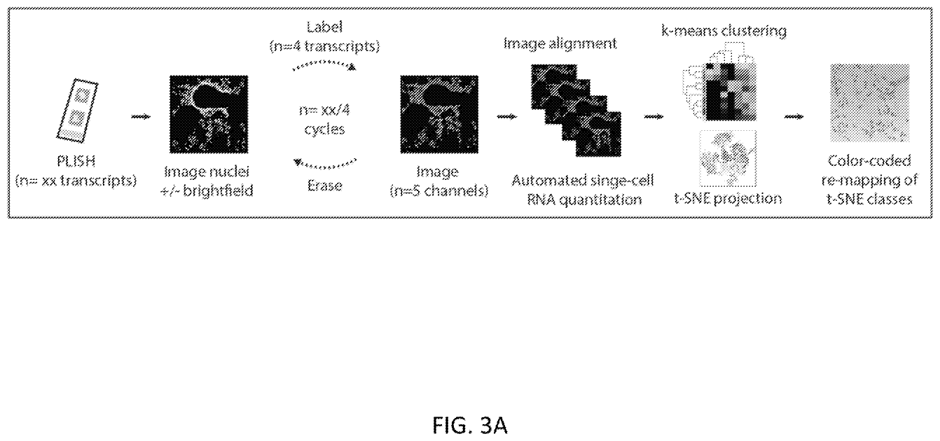

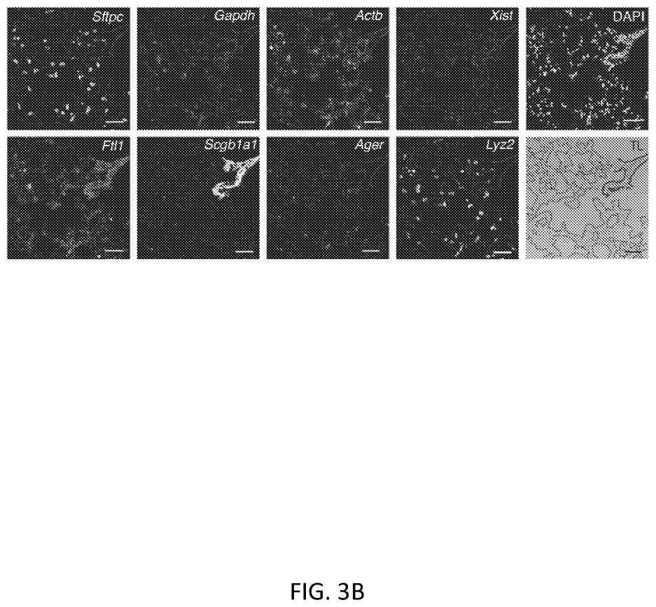

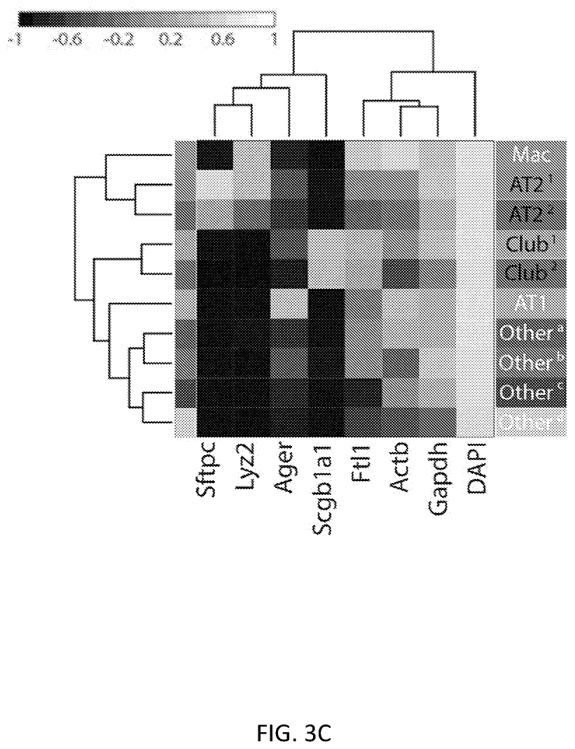

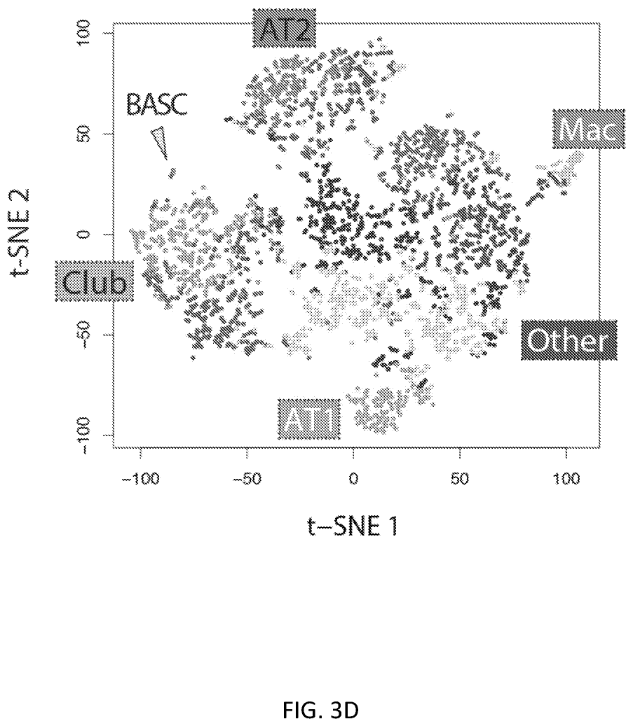

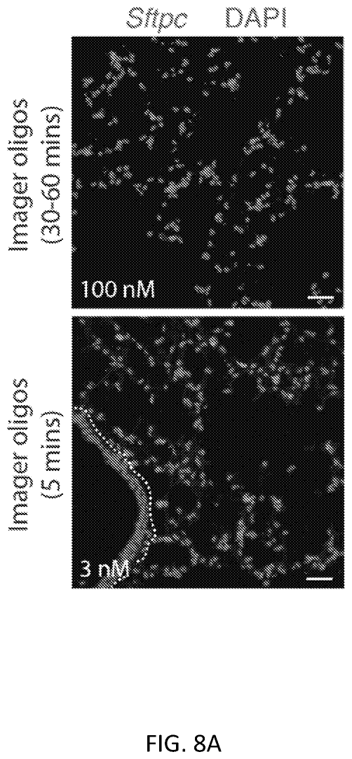

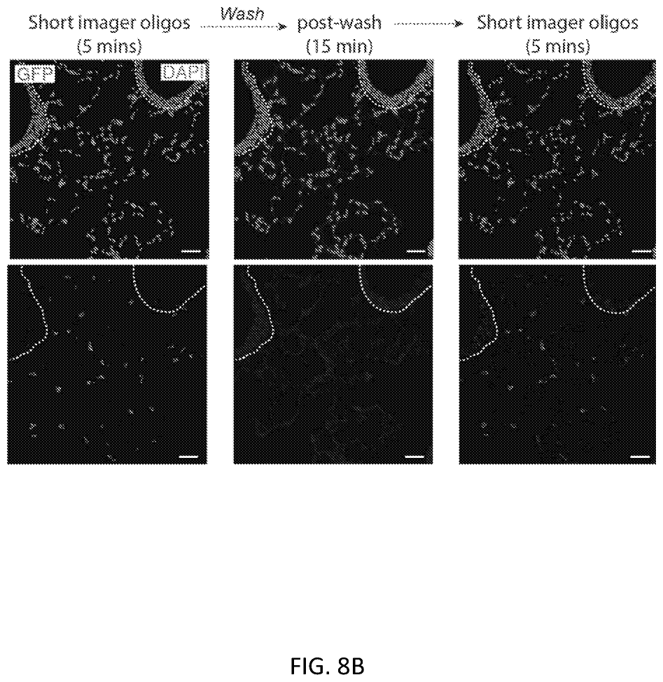

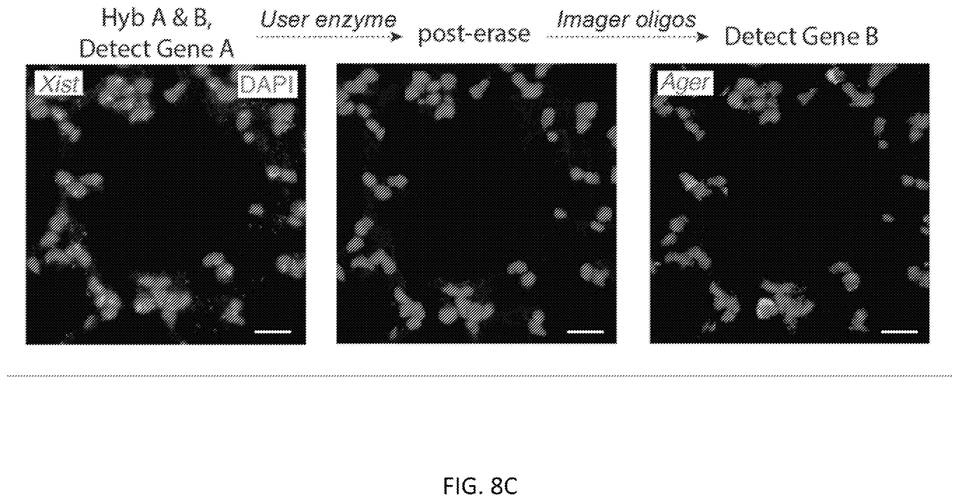

Highly multiplexed measurement of different RNA species requires iterated data collection cycles, since conventional fluorescence microscopy only provides up to five channels (FIG. 3A). The data collection cycles include fluorescent labeling of a subset of the ‘barcodes’ (i.e., unique nucleotide sequences complementary to fluorescently labeled ‘imager’ oligonucleotides) in a sample, imaging of the labeled transcripts, and erasure of the fluorescent signal. Ideally, the cycles should be fast, and the erasure should not cause any mechanical or chemical damage to the sample. Consistent with prior work, we found that PLISH puncta could be imaged in the presence of a 3 nM background of freely-diffusing imager oligonucleotides (FIG. 8A and Blab et al. (2004) Analytical Chemistry 76:495-498). This allowed us to streamline data collection by eliminating a wash step, and also presented a simple erasure strategy. By using short imager oligonucleotides that equilibrate rapidly on and off of RCA amplicons (Jungmann et al. (2014) Nature Methods 11:313-318), we could erase fluorescence from a previous cycle by a simple buffer exchange (FIG. 8B). We also established an erasure method based on uracil-containing imager oligonucleotides, which were removed with a 15 minutes enzymatic digestion (FIG. 8C). Thus, we could image PLISH puncta in five different color channels with spectrally-distinct fluorophores, and we were able to complete cycles in as little as 20 minutes, which approaches the cycle time of an Illumina MiSeq instrument (support.illumina.com).

To demonstrate and validate the multiplexing capacity of PLISH, we co-localized the mRNA of eight selected genes in 2900 single cells from an adult mouse lung (FIG. 3B). Our panel included four commonly used lung cell type markers that have been previously characterized, and four ubiquitously expressed genes. The targeted transcripts were Sftpc (AT2 cells), advanced glycosylation end product-specific receptor (Ager, AT1 cells), Scgb1a1 (Club cells), Lyz2 (macrophage and AT2 cell subset), ferritin light polypeptide 1 (Ftl1), beta actin (Actb), inactive X specific transcripts (Xist), and glyceraldehyde-3-phosphate dehydrogenase (Gapdh). The eight RNA species were barcoded in a single PLISH reaction, and the data were collected with a pair of label-image-erase cycles using the enzymatic erasure approach described above (FIG. 3A). A nuclear counterstain (DAPI) and transmitted light micrograph were also obtained.

To quantify the expression of all eight genes on a per-cell basis, we created a PLISH-specific pipeline in CellProfiler, an open-source software package (Kamentsky et al. (2011) Bioinformatics 27:1179-1180). The pipeline first identified nuclei in the DAPI channel, which were used as anchor points for expansion to full-cell assignments. Fortuitously, the bulk of the detected mRNAs in AT1 cells, which have an extremely flat and broad morphology, were clustered around the nuclei. We summed the PLISH signal for each gene in the nuclear and peri-nuclear regions of each cell, and saved the results as single-cell expression profiles indexed on anatomical location. We also created a utility to pseudocolor cells in a transmitted light micrograph according to their inferred cell type (see below), so that we could visualize the relationship between cellular gene expression and anatomical localization.

Automated Cell Classification and Insights into Lung Biology

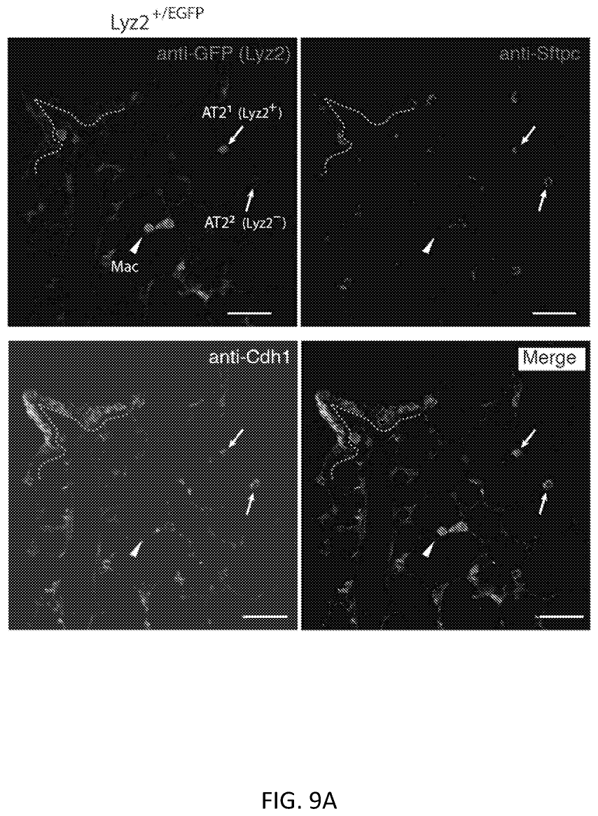

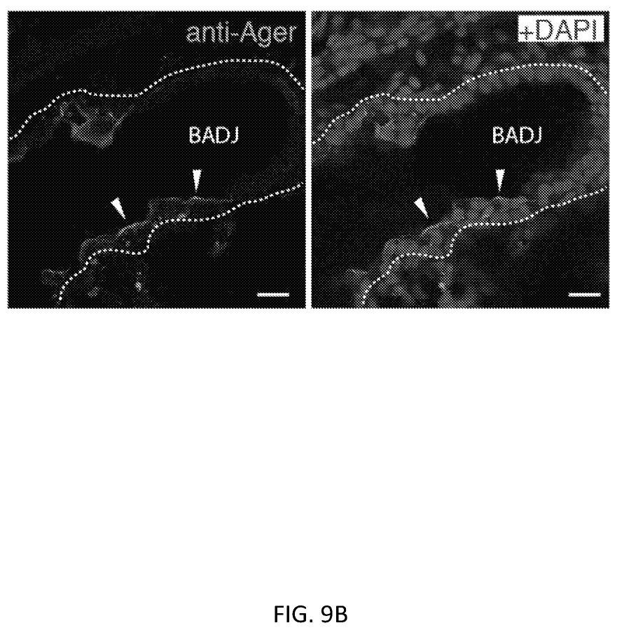

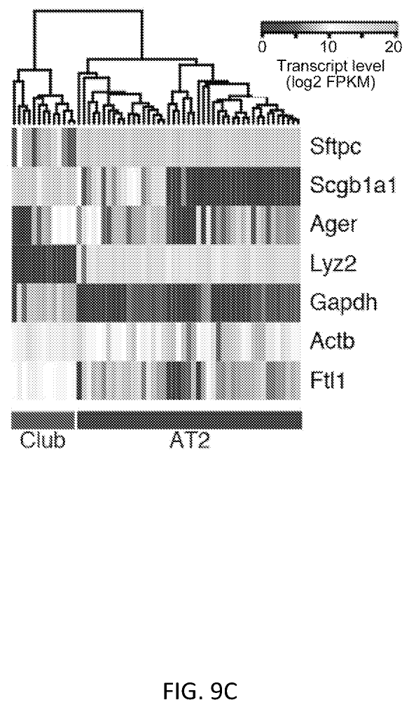

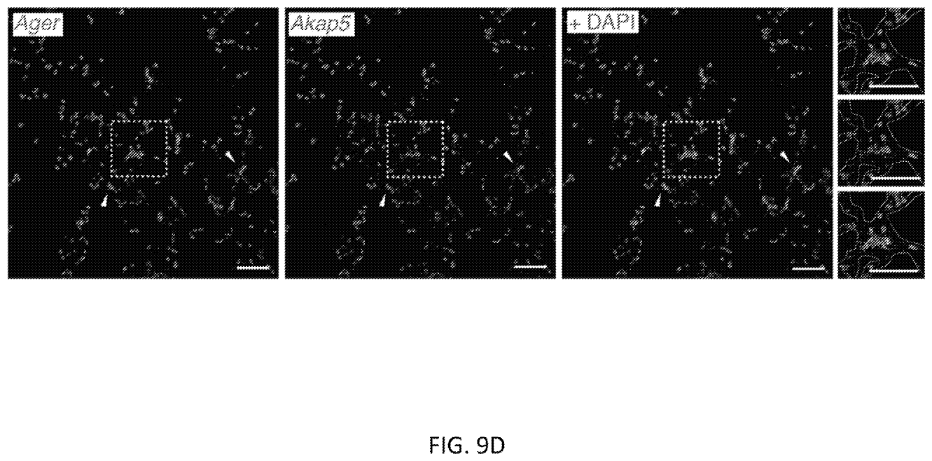

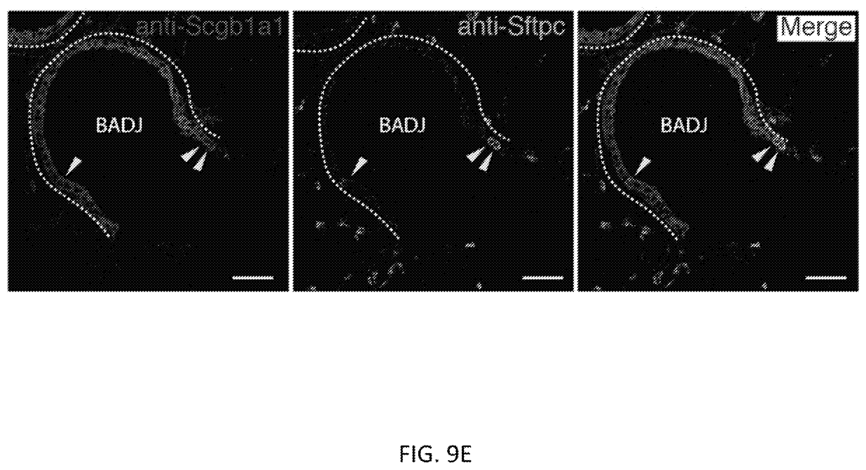

An important scientific challenge is to identify and map all of the molecularly distinct cell types that make up complex tissues, and in situ single-cell profiling should be a powerful tool for working towards this goal. As a proof-of-concept for this, we asked whether known lung cell types could be rediscovered by an automated and unsupervised analysis of our multiplexed PLISH data set. We used two standard data analysis tools, K-means clustering (FIG. 3C) and t-distributed stochastic neighbor embedding (van der Maaten and Hinton, (2008) Journal of Machine Learning Research: JMLR 9:2579-2605) (t-SNE, FIG. 3D), to classify and visualize the entire population of cells. The automated analysis identified ten cell classes, four of which were labeled ‘other’ because they were defined primarily by ‘signature’ profiles of ubiquitously-expressed genes. The remaining six classes were associated with a known lung cell type based on marker-gene expression. The Sftpc and Scgb1a1 positive cell classes were labeled as AT2 and Club, respectively, while the Lyz2 positive class was labeled as macrophage (one of the two AT2 cell classes was also Lyz2 positive as previously reported in (Desai et al., (2014) Nature 507:190-194), FIG. 9A). The cell class with the highest Ager expression was labeled as AT1, but Ager mRNA was also detected in a subset of AT2 and Club cells, and in one of the four ‘other’ cell classes, indicating it is not particularly specific for AT1 cells. We validated the PLISH results by indirect immunohistochemistry (FIG. 9B) and by comparison with previously published scRNA-seq data (FIG. 9C), which confirmed the low specificity of Ager for AT1 cells. We also analyzed the RNA expression of Akap5, another transcript that is highly-enriched in AT1 cells (Treutlein et al. (2014) Nature 509:371-375), and found that its localization correlated closely with Ager's (FIG. 9D).

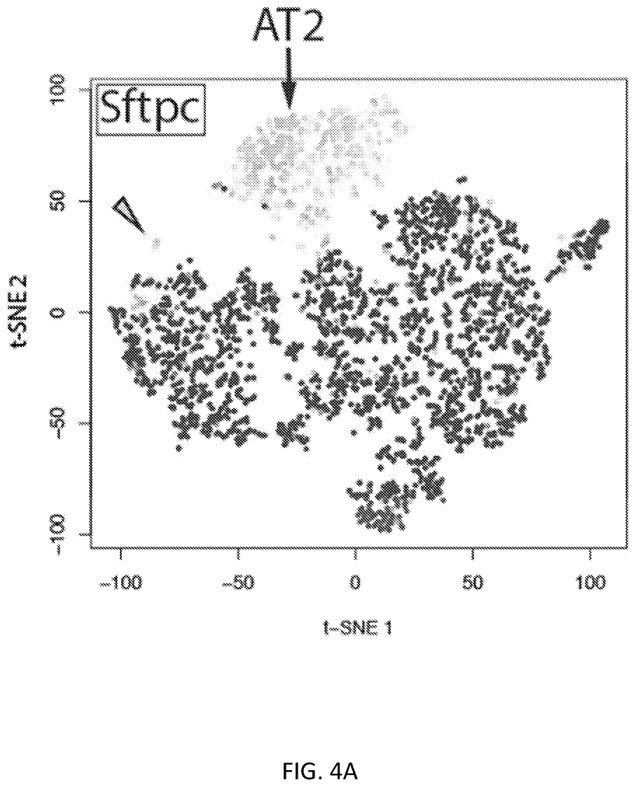

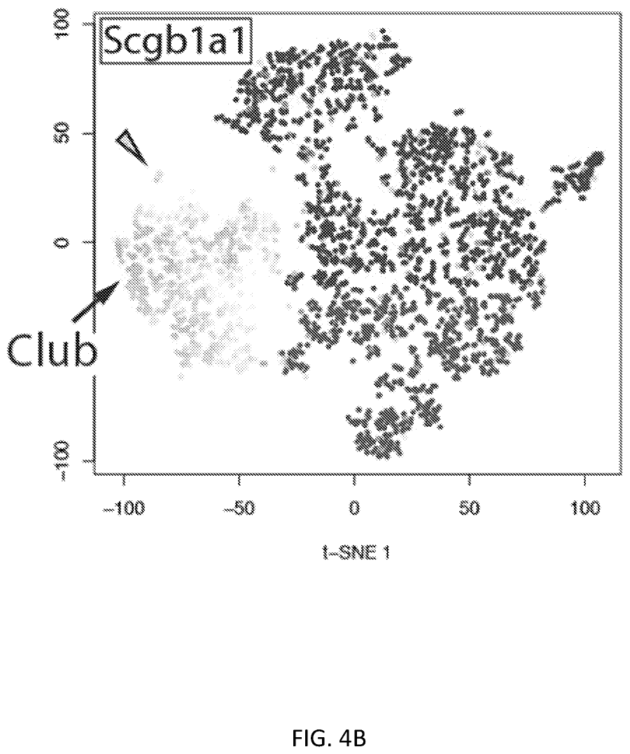

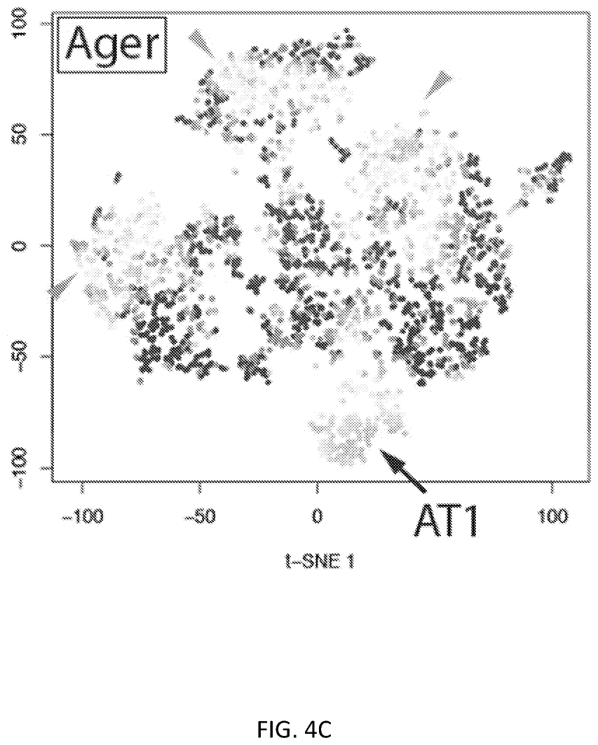

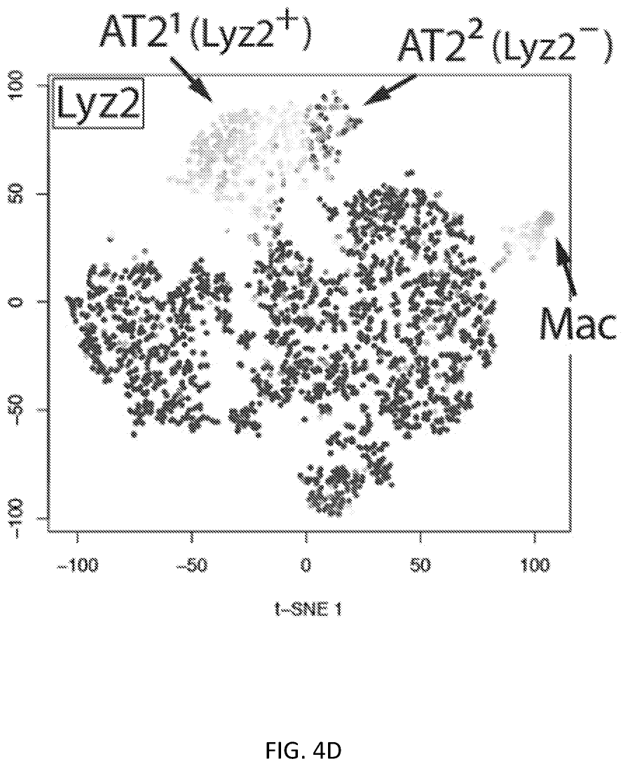

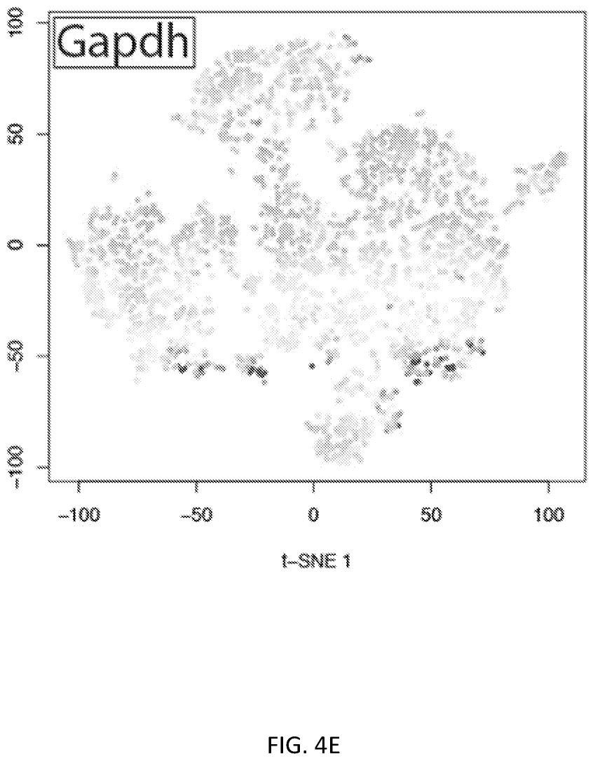

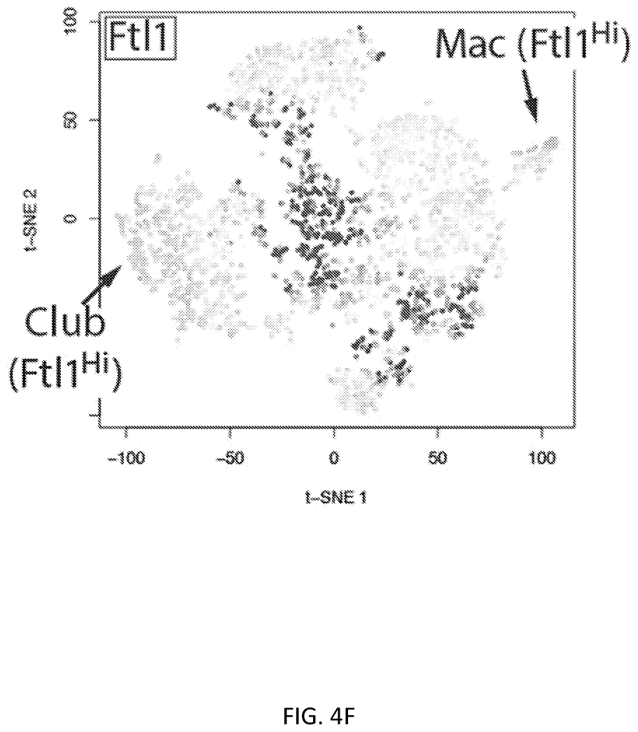

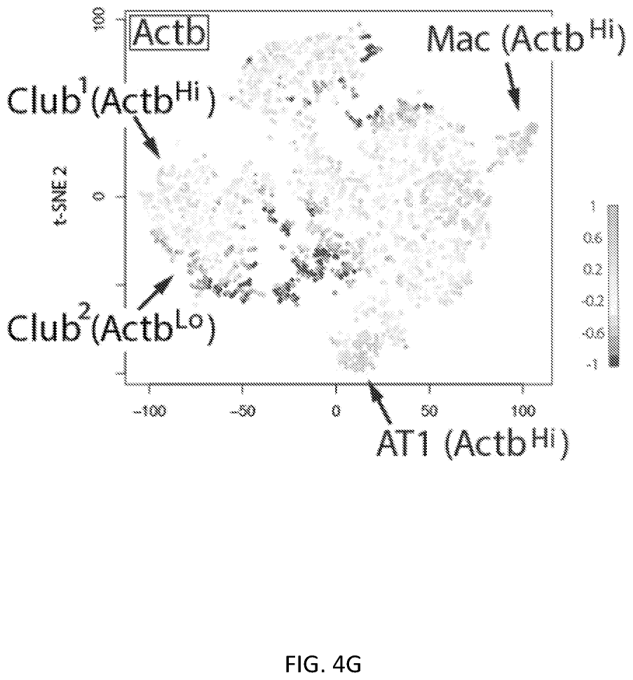

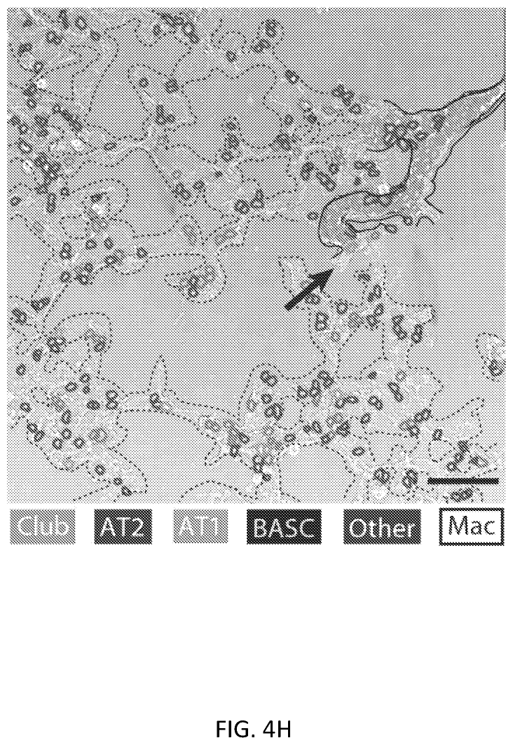

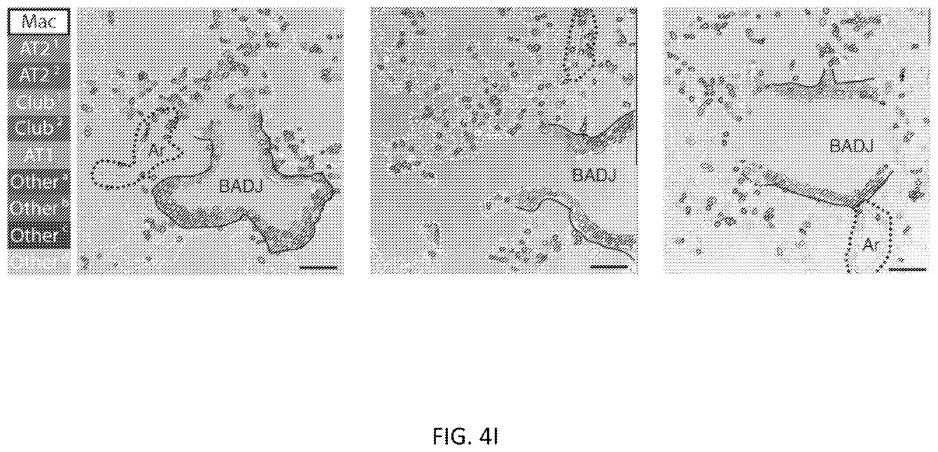

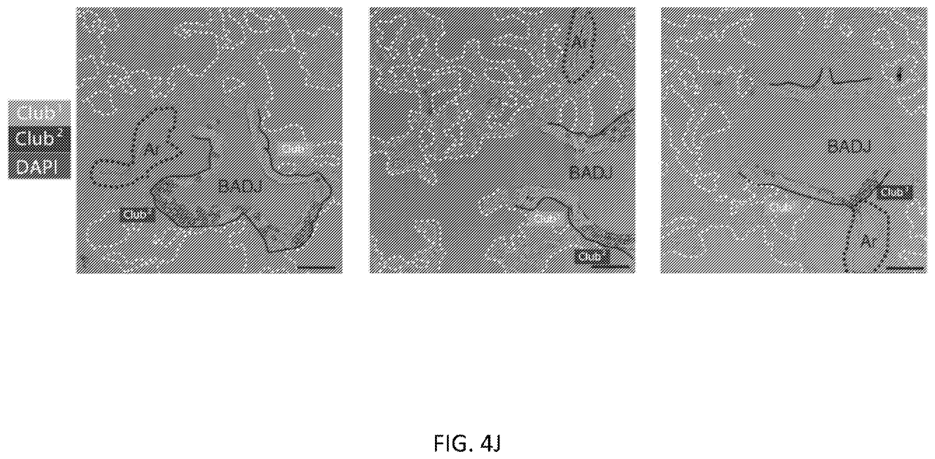

For a higher-resolution analysis of cellular gene expression, we examined the expression pattern of individual genes in re-colored t-SNE plots (FIGS. 4A-4D). We found a small cloud of cells between the Club and AT2 clusters that expressed both Sftpc and Scgb1a1. On the basis of this dual expression, we assigned them as the BASC type (Kim et al. (2005) Cell 121:823-835). We also noted that Lyz2 expression partitioned the AT2 cells into two classes designated Lyz2⁺ and Lyz2⁻, while Actb segregated Club cells into two classes designated Actb^{Hi} and Actb^{Lo}. Gapdh was the most uniformly expressed transcript, consistent with its role as a ‘housekeeping’ gene (FIGS. 4E-4G). Ftl1 expression was highest in alveolar macrophages, as expected, where it is believed to play a role in processing iron from ingested red blood cells (McGowan and Henley (1988) The Journal of Laboratory and Clinical Medicine 111:611-617). Unexpectedly, Ftl1 was also highly expressed in Club cells. Actb expression was highest in macrophages, presumably because of its functional role in motility, and in AT1 cells, which must maintain a flat morphology and expansive cytoskeleton (Foster et al. (2010) Pediatric Research 67:585-590).

To validate the PLISH results, we pseudocolored the cells in transmitted-light images according to their class (FIGS. 4H-4I). Importantly, no spatial information was included in the k-means clustering. Several observations confirmed the accuracy of the automated classification. First, the Club cell class mapped perfectly onto the bronchial epithelium, while cells from the AT1 and AT2 classes were distributed throughout the alveolar compartment. The rare BASCs also localized precisely to the bronchioalveolar junctions, where they have been shown to reside by immunostaining (Kim et al. (2005) Cell 121:823-835) (FIG. 4H and FIG. 9J). The macrophage class was primarily found inside the alveolar lumen, and many exhibited a characteristic rounded cell shape. The Other^{d} class of cells was enriched in pulmonary arteries, and therefore might represent endothelial or perivascular cells. We further observed a striking spatial segregation of the two Club cell classes. Actb^{Hi} Club cells clustered together at the bronchial terminus, while Actb^{Lo} Club cells populated more proximal domains (FIGS. 4I-4J). While the significance of this pattern is not immediately obvious, it emphasizes how PLISH can readily integrate molecular and spatial features of single cells to generate insights that would be missed with either piece of information alone.

Discussion

PLISH represents a practical technology for multiplexed expression profiling in tissues. It combines high performance in four key areas: specificity, detection efficiency, signal-to-noise, and speed. The specificity derives from coincidence detection, which requires two probes to hybridize next to one another for signal generation. Efficient detection of low-abundance transcripts is accomplished by targeting multiple sites along the RNA sequence. Enzymatic amplification produces extremely bright puncta and allows many different RNA transcripts to be marked with unique barcodes in one step. The different RNA transcripts can then be iteratively detected to rapidly generate high dimensional data.

While low-plex PLISH on a handful of different genes can be valuable, the PLISH technology is also scalable, without requiring specialized microscopes (or other equipment), software, or computational expertise. The oligonucleotides and enzymes are inexpensive and commercially available from multiple vendors. The H probes are the cost-limiting reagent, but can be synthesized in pools (Murgha et al. (2014) PLoS One 9:e94752; Beliveau et al. (2012) PNAS 109:21301-21306). Assuming five pairs of H probes for each target RNA species, and 20 cents for a 40 mer oligonucleotide, the cost of PLISH reagents amounts to $3 per gene. It should therefore be practical to simultaneously interrogate entire molecular systems, such as signaling pathways or super-families of adhesion receptors. The high specificity and signal-to-noise of PLISH will be advantageous for deep profiling, where non-specific background increases with increasingly complex mixtures of hybridization probes (Moffitt et al. (2016) PNAS 113:11046-11051).

Our initial studies demonstrate PLISH's capacity for rapid, automated and unbiased cell-type classification, and illustrate how it can complement single-cell RNA sequencing (sc-RNAseq). Sc-RNAseq offers greater gene depth than in situ hybridization approaches, but it is less sensitive, fails to capture spatial information, and induces artefactual changes in gene expression during tissue dissociation (van den Brink et al. (2017) Nature Methods 14:935-936; Lee et al. (2015) Nature Protocols 10:442-458). PLISH provides the missing cytological and spatial information, and it is applied to intact tissues. Going forward, sequencing can be used to nominate putative cell types and molecular states based on the coordinate expression of ‘signature genes’, and multiplexed PLISH can be used to distinguish true biological variation from technical noise and experimentally-induced perturbations. Importantly, multiplexed PLISH provides the tissue context of distinct cell populations, which is essential for understanding the higher-order organization of intact systems like solid tumors and developing organs. In diseases like IPF where morphology and gene expression are severely deranged (Xu et al. (2016) JCI Insight 1:e90558), histological, cytological and spatial features may even be essential for making biological sense of sequencing data.

Currently, efforts are underway to more deeply characterize cellular states by integrating diverse types of molecular information. We have already demonstrated the combined application of PLISH with conventional immunostaining. Going one step further, oligonucleotide-antibody conjugates make it possible to mix and match protein and RNA targets in a multiplexed format (Weibrecht et al. (2013) Nature Protocols 8:355-372). The generation of comprehensive, multidimensional molecular maps of intact tissues, in both healthy and diseased states, will have a fundamental impact on basic science and medicine.

Materials and Methods

Materials

Unless otherwise specified, all reagents were from Thermo-Fisher and Sigma-Aldrich. Oligonucleotides were purchased from Integrated DNA Technologies. T4 polynucleotide kinase, T4 ligase, USER enzyme and their respective buffers were purchased from New England Biolabs. Nxgen phi29 poly-merase and its buffer were purchased from Lucigen.

Abbreviations: BSA, bovine serum albumin; DAPI, 4,6-diamidino-2-phenylindole; DEPC, diethyl-pyrocarbonate; EDTA, ethylenediaminetetraacetic acid; min, minutes; PBS, phosphate buffered saline; PFA, paraformaldehyde; RCA, rolling circle amplification; RT, room temperature. All oligonucleotide sequences are listed in Table 1.

Sample Preparation

HCT116 cells (ATCC; CCL-247) were authenticated by HLA typing and confirmed negative for Mycoplasma contamination using PCR. Cells were grown on poly-lysine coated #1.5 coverslips (Fisher-brand 12-544 G) using standard cell culture protocols until they reached the desired confluency. The cells were rinsed in 1× PBS and fixed in 3.7% formaldehyde with 0.1% DEPC at RT for 20 minutes. The fixed cells were treated with 10 mM citrate buffer (pH 6.0) at 70° C. for 30 minutes, dehydrated in an ethanol series, then enclosed by application of a seal chamber (Grace Biolabs 621505) to the coverslip.

Lungs were collected from adult B6 mice (Jackson Labs) and fixed by immersion in 4% PFA as previously described (Desai et al. (2014) Development 143:3632-3637). Non-IPF human lung tissue was obtained from a surgical resection, and IPF tissue from an explant. All mouse and human research were approved by the Institutional Animal Care and Use Committee and Internal Review Board, respectively, at Stanford University. The tissues were fixed by immersion in 10% neutral buffered formalin in PBS at 4° C. overnight under gentle rocking, cryoprotected in 30% sucrose at 4° C. overnight, submerged in OCT (Tissue Tek) in an embedding mold, frozen on dry ice, and stored at −80° C. 20 mm sections were cut on a cryostat (LeicaCM 3050S) and collected on either poly-lysine coated #1.5 coverslips or glass slides (Fisherbrand Superfrost), air dried for 10 minutes, and post-fixed with 4% PFA at RT for 20 minutes. The human lung tissue in FIG. 2A was formalin-fixed and paraffin-embedded (FFPE) according to standard protocols, and 20 mm sections were cut on a microtome and collected on glass slides. The FFPE sections were deparaffinized by immersion in Histoclear (National Diagnostics, HS-200) for 3×5 minutes, then dehydrated in an ethanol series and post-fixed with 4% PFA at RT for 20 minutes. Tissue sections were treated with 10 mM citrate buffer (pH 6.0) containing 0.05% lithium dodecyl sulfate at 70° C. for 30 minutes, or in some experiments, with 0.1 mg/ml Pepsin in 0.1M HCl for 8 minutes at 37° C. and dehydrated in an ethanol series. Following treatment, sections were air dried for 10 minutes and enclosed by application of a seal chamber.

PLISH Probe Design and Preparation

Target RNAs were probed at ˜40 nucleotide detection sites, with 1 to 10 sites per RNA species depending on expression level. NCBI BLAST searches were used to eliminate detection sites that shared 10 or more contiguous nucleotides with a non-target RNA. The detection sites were also selected to minimize self-complementarity as indicated by the IDT oligo analyzer. Each detection site was targeted with a pair of H probes designated HL (left H probe) and HR (right H probe). The HL and HR probes included ˜20 nucleotide binding sequences that were complementary respectively to the 5′ and 3′ halves of the detection site. The binding sequences were chosen so that the 5′ end of the HL binding sequence and the 3′ end of the HR binding sequence would abut at a 5′-AG-3′ or a 5′-TA-3′ dinucleotide in the target RNA. The lengths of the binding sequences were adjusted so that the melting temperature of the corresponding DNA duplex would fall between 45-65° C. as computed by IDT Oligo analyzer using default settings of 0.25 mM oligo concentration and 50 mM salt concentration. To generate H probes, suitable HL and HR binding sequences were catenated at their respective 5′ and 3′ ends with overhang sequences taken from one of eight modular design templates (Table 1). The left and right overhang sequences in each design template were complementary to a specific bridge (B) and circle (C) oligonucleotide, which directed a desired fluorescent readout. The design templates reported here utilized a common 31 base oligonucleotide for the bridge. Following previous work (Soderberg et al., supra), the circle oligonucleotides were ˜60 bases long with 11 base regions of complementarity to cognate H probes on either end. The circle sequences were chosen to minimize self-complementarity. Each imager oligonucleotide was complementary to a barcode embedded in one of the C oligonucleotides, allowing unique detection of the corresponding RCA amplicon.

The H-probe oligonucleotides were ordered on a 25 nanomole scale with standard desalting. The B and C oligonucleotides were ordered on a 100 nanomole scale with HPLC purification and phosphorylated with T4 polynucleotide kinase according to the manufacturer's recommendations. Imager oligonucleotides were purchased either as HPLC-purified fluorophore conjugates (A488, Texas Red, Cy3, Cy5), or as amine-modified oligonucleotides that were subsequently coupled to Pacific Blue-NETS ester according to the manufacturer recommendations.

PLISH Barcoding Procedure

Six buffers were used for PLISH barcoding: H-probe buffer (1M sodium trichloroacetate, 50 mM Tris pH 7.4, 5 mM EDTA, 0.2 mg/mL Heparin), bridge-circle buffer (2% BSA, 0.2 mg/mL heparin, 0.05% Tween-20, 1× T4 ligase buffer in RNAse-free water), PBST (PBS+0.1% Tween-20), ligation buffer (10 CEU/μl T4 DNA ligase, 2% BSA, 1× T4 ligase buffer, 1% RNaseOUT and 0.05% Tween-20 in RNAse-free water), labeling buffer (2×SSC/20% formamide in RNAse-free water), and RCA buffer (1 U/μl Nxgen phi29 polymerase, 1× Nxgen phi29 polymerase buffer, 2% BSA, 5% glycerol, 10 mM dNTPs, 1% RNaseOUT in RNAse-free water).

An H cocktail was prepared by mixing H probes in H-probe buffer at a final concentration of 100 nM each. If an RNA was targeted with more than five probe sets, the concentrations of the H probes for that RNA were pro-rated so that their sum did not exceed 1000 nM. A BC cocktail was also prepared by mixing B and C oligonucleotides in bridge-circle buffer at a final concentration of 6 μM each.

Single-step barcoding was performed in sealed chambers. The workflow consisted of three steps: (i) The sample was incubated in the H cocktail at 37° C. for 2 hours. The sample was then washed 4 x 5 minutes with H-probe buffer at RT, and incubated in the BC cocktail at 37° C. for 1 hour. (ii) Following a 5 minutes wash with PBST at RT, the sample was incubated in ligation buffer at 37° C. for 1 hour. (iii) The sample was washed 2×5 minutes with labeling buffer at RT, and washed with 1× Nxgen phi29 polymerase buffer at RT for 5 minutes. The sample was then incubated in RCA buffer at 37° C. for 2 hours (typical for cultured cells) to overnight (typical for tissue). Finally, the sample was washed 2×5 minutes with labeling buffer.

Imaging

Barcoded PLISH samples were fluorescently labeled by two different procedures, designated ‘washout’ and ‘fast’. In the washout procedure, the sample was incubated with imager oligonucleotides in imager buffer (labeling buffer with 0.2 mg/mL heparin) at a final concentration of 100 nM each for 30 minutes, and then washed 2×5 minutes with PBST at RT. In the fast procedure, the sample was incubated for 5 minutes with imager oligonucleotides in imager buffer at a final concentration of 3 nM each, and then imaged immediately. Samples that did not require label-image-erase cycles were stained with DAPI (stock 1 mg/ml; final concentration—1:1000 in PBS) for 5 minutes and mounted in H-1000 Vec-tashield mounting medium (Vector).

Data were collected by confocal microscopy (Leica Sp8 and Zeiss LSM 800) using a 40× oil immersion or a 25× water immersion objective lens. 20 μm z-stacks were scanned, and maximum projection images were saved for analysis. For 5-color experiments, DAPI was added after the Pacific Blue channel had been imaged, and the Texas Red and Cy3 channels were linearly unmixed using Zeiss software. Transmitted light images were acquired on a Leica Sp8 confocal microscope using the 488 nm Argon laser and the appropriate PMT-TL detector. Images from serial rounds of data collection were aligned using the nuclear stain from each round as a fiducial marker. Unless otherwise stated, imaging data of cells and mouse lung tissue are representative of three independent experiments with ˜4 fields of view each. Imaging data of human lung tissue are representative of two independent experiments with ˜4 fields of view each.

PLISH and HCR Co-Localization

HCR was performed following a published protocol (Choi et al., supra) with probes that targeted two sites covering nucleotides 621-670 and 1159-1208 in the mouse Axin2 transcript, and Alexa-Fluor 488-/AlexaFluor 647-labeled amplifier oligonucleotides. The samples were then processed for PLISH with H probes targeting four sites covering nucleotides 347-386, 1878-1917, 2412-2451 and 2956-2995 in the Axin2 transcript, and imaged using a Cy3-labeled imager oligonucleotide.

PLISH with Concurrent Immunohistochemistry

PLISH barcoding was performed as described above. Subsequently, the sample was washed 3×5 minutes with PBST at RT, and incubated in blocking solution (50 ml/ml [5%] normal goat serum, 1 ml/ml [0.1%] Triton X-100, 5 mM EDTA and 0.03 g/ml [3%] BSA in PBS) at RT for 1 hour. The sample was then incubated with primary antibody (Rabbit anti-pro-Sftpc, Millipore, 1:500 or Rabbit anti-Cytokeratin 5, Abcam Ab193895, 1:400) in blocking solution at 37° C. for 2 hours under gentle rocking, washed 4×5 minutes with PBST at RT, and incubated with secondary antibody (Goat anti-Rabbit-Cy5, Jackson Lab, 1:250) and DAPI (1:1000) in blocking solution at RT for 1 hour. The sample was washed 3×5 minutes in PBST at RT and mounted in H-1000 Vectashield.

Antisense Blocking Oligonucleotide

Mouse lung tissue cryosections were collected on slides, post-fixed and processed as described above. The samples were incubated with a 60-base oligonucleotide complementary to nucleotides 219-278 in the Scgb1a1 mRNA, or with a scrambled 60-base oligonucleotide, at 100 nM final concentration in H-probe buffer at 37° C. for 2 hours. The samples were then washed 2×5 minutes with H-probe buffer at RT, and processed for PLISH using H probes that targeted nucleotides 229-268 in the Scgb1a1 transcript.

Signal Erasure for Iterative Cycles of PLISH

To perform enzymatic erasure, 15-20 base imager oligonucleotides were ordered with the dT nucleotides replaced by dU nucleotides. Following imaging, the signal was erased by incubating the sample with 0.1 U/mL USER enzyme in 1× USER enzyme buffer at 37° C. for 20 minutes, followed by washing 2×3 minutes with PBST at RT. To perform rapid erasure, short 10-11 base oligonucleotides were ordered. Following imaging, the signal was erased by incubating the sample with PBST at 37° C. for 15 minutes.

Correlative Immunostaining

Lungs collected from B6 and the Lyz2^{+/EGFP} mouse strains (Faust et al., (2000) Blood 96:719-726) were fixed and immunostained as whole mounts as previously described (Desai et al., supra). Primary antibodies were chicken anti-GFP (Abcam ab13970), rat anti-Ecad/Cdh1 (Invitrogen ECCD-2), goat anti-Scgb1a1 (gift from Barry Stripp), rabbit anti-pro-Sftpc (Chemicon AB3786), and rat anti-Ager (R and D MAB1179). Fluorophore-conjugated secondary antibodies raised in Goat (Invitrogen) or Donkey (Jackson Labs) were used at 1:250 and DAPI at 1:1000.

Data Analysis

FIJI was used to pseudocolor unprocessed micrographs for display as three-color overlays. A custom CellProfiler (Kamentsky et al. (2011) Bioinformatics 27:1179-1180) pipeline was created to measure RNA signal intensities at the single-cell level. Briefly, the centers of cell nuclei were first identified as maxima in a filtered DAPI image, and associated with a numerical index. Nuclear boundaries were assigned by a propagation algorithm, and then expanded by ˜1 micron to define sampling areas. The following data were then recorded: (i) average pixel intensities for each data channel over each sampling area; (ii) the coordinates of the sampling areas; (iii) shape metrics for the corresponding nuclei; and (iv) an image with the boundary pixels of each nucleus set equal to the associated index value. For each RNA species, the PLISH data were first normalized onto a 0:10 scale by dividing through by the largest value observed in any cell over all of the fields of view, and then multiplying by ten. The data were then log-transformed onto a −1:1 scale by the operation: transformed_data=log(0.1+normalized_data). Custom Matlab scripts were used to perform hierarchical clustering of the log-transformed single-cell expression profiles, to generate heatmaps, and to create images with the boundary pixels of each nucleus colored according to a cluster assignment. Custom R scripts were used for k-means clustering and to make t-SNE projection plots.

TABLE 1
Oligonucleotide Reagents for PLISH
Connector circles

| | |
|---|---|
| CC06-DP4 | TTCCTGACCTAACAAAACATGCGTCTATTTAGTGGAGCCACA |
| | TAATTATAACCTGGCTAT (SEQ ID NO: 1) |
| CC03 -X1 | TCGAACGAATAACAAAGGGAGACTGATTACACGATAACAAC |
| | AATACACACAATACGTCGG (SEQ ID NO: 2) |
| CC02-Y1 | TAGACGTACGAGATTAAAACATAGAGGCACTTGAGCTAGAAT |
| | CATATAACAGGTTGTTCT (SEQ ID NO: 3) |
| CC04-X4 | TCGCCTACTAAACAAAGAACCTCCATTAACATCGCAACAACA |
| | ATACACACGCACTTATGT (SEQ ID NO: 4) |
| CC05-DP5 | TGTTAGCGCTAACAAAATGCTGCTGCTGTACTACGAACAACA |
| | ATACACATGTTACGACGT (SEQ ID NO: 5) |
| CC07-Y7 | GACAATTACACGACTAAAACATCTCGGTGCTTGAACTGGTCT |
| | CAACTAACCTGAGGAATA (SEQ ID NO: 6) |
| CC11X-X8 | TAACACGAACACTAAACAAGTCTA ACT AACGAT |
| | ACCATAAAGAAGAATAACCGAATCAAGAT (SEQ ID NO: 7) |
| CC12X-X9mod | ACCATACTTGAACAAACCACACTCACATGGATCCACTCGTAA |
| | CAACAATAACAGGATACATC (SEQ ID NO: 8) |

Common Bridge

| | |
|---|---|
| CB2-31AC | TCAACTCGACGTATAACATAACGACGTAAGT (SEQ ID NO: 9) |

Detection oligonucleotides

| | |
|---|---|
| AF488-Y1 | /5Alex488N/AGAGGCACTTGAGCTAGAAT |
| | (SEQ ID NO: 10) |
| Cy5-DP5 | /5Cy5/AATGCTGCTGCTGTACTACGG |
| | (SEQ ID NO: 11) |
| X1-Cy3 | GGGAGACTGATTACACGATA/3Cy3Sp/ |
| | (SEQ ID NO: 12) |
| TEX615-X4 | /5TEX615/AGAACCTCCATTAACATCGC |
| | (SEQ ID NO: 13) |

Detection oligos with deoxy-U

| uY7-A488 | /5Alex488N/CUCGGUGCUUGAACUGGUCU | | |
|---|---|---|---|
| | (SEQ ID NO: 14) | | |
| uX8-Cy3 | GUCUAACUAACGAUACCAUA/3Cy3Sp/ | | |
| | (SEQ ID NO: 15) | | |
| uX9-Texas red | /5Tex615/CUCACAUGGAUCCACUCGU | | |
| | (SEQ ID NO: 16) | | |
| uDP4-Cy5 | /5Cy5/AUGCGUCUAUUUAGUGGAGCC | | |
| | (SEQ ID NO: 17) | | |
| | | H Probes (2X, 3X, | Design Templates for H Probes |
| | | 4X, 5X, 6X,7X,11X, | (2X, 3X, 4X, 5X, 6X, |
| Fluorophore | Compatible CC | 12X) | 7X, 11X, 12X) |
| DP5- Cy5 | CC05-DP5 + | HL5X-Gene-Position | TAGCGCTAACAACTTACGTCGTTATG: LLLLL |
| | CB2-31AC | | (SEQ ID NO: 18) |
| | | HR5X-Gene-Position | RRRRR: TTATACGTCGAGTTGAACGTCGTAACA |
| | | | (SEQ ID NO: 19) |
| Y1-A488 | CC02-Y1 + CB2- | HL2X-Gene-Position | TCGTACGTCTAACTTACGTCGTTATG: LLLLL |
| | 31AC | | (SEQ ID NO: 20) |
| | | HR2X-Gene-Position | RRRRR: TTATACGTCGAGTTGAAGAACAACCTG |
| | | | (SEQ ID NO: 21) |
| X1-Cy3 | CC03-X1 + CB2- | HL3X-Gene-Position | TATTCGTTCGAACTTACGTCGTTATG: LLLLL |
| | 31AC | | (SEQ ID NO: 22) |
| | | HR3X-Gene-Position | RRRRR: TTATACGTCGAGTTGACCGACGTATTG |
| | | | (SEQ ID NO: 23) |
| X4- Texas Red | CC04-X4 + CB2- | HL4X-Gene-Position | TTAGTAGGCGAACTTACGTCGTTATG: LLLLL |
| | 31AC | | (SEQ ID NO: 24) |
| | | HR4X-Gene-Position | RRRRR: TTATACGTCGAGTTGAACATAAGTGCG |
| | | | (SEQ ID NO: 25) |
| uDP4-Cy5 | CC06-DP4 + | HL6X-Gene-Position | TAGGTCAGGAAACTTACGTCGTTATG: LLLLL |
| | CB2-31AC | | (SEQ ID NO: 26) |
| | | HR6X-Gene-Position | RRRRR: TTATACGTCGAGTTGAATAGCCAGGTT |
| | | | (SEQ ID NO: 27) |
| uY7-A488 | CC07-Y7 + CB2- | HL7X-Gene-Position | GTGTAATTGTCACTTACGTCGTTATG: LLLLL |
| | 31AC | | (SEQ ID NO: 28) |
| | | HR7X-Gene-Position | RRRRR: TTATACGTCGAGTTGATATTCCTCAGG |
| | | | (SEQ ID NO: 29) |
| uX8- Cy3 | CC11X-X8 + CB2- | HL11X-Gene-Position | TGTTCGTGTTAACTTACGTCGTTATG: LLLLLLL |
| | 31AC | | (SEQ ID NO: 30) |
| | | HR11X-Gene-Position | RRRRRR: TTATACGTCGAGTTGAATCTTGATTCGGT |
| | | | (SEQ ID NO: 31) |
| uX9-Texas Red | CC12X-X9mod + | HL12X-Gene-Position | TCAAGTATGGTACTTACGTCGTTATG: LLLLL |
| | CB2-31AC | | (SEQ ID NO: 32) |
| | | HR12X-Gene-Position | RRRRR: TTATACGTCGAGTTGAGATGTATCCTG |
| | | | (SEQ ID NO: 33) |

Mouse - PLISH probes

| | |
|---|---|
| mm-HL4X-Ager-1335 | TTAGTAGGCGAACTTACGTCGTTATGTCTGGTTGGA |
| | GAAGGAAGTG (SEQ ID NO: 34) |
| mm-HR4X-Ager-1335 | AGCATGGATCATGTGGGCTTATACGTCGAGTTGAAC |
| | ATAAGTGCG (SEQ ID NO: 35) |
| mm-HL4X-Ager-1280 | TTAGTAGGCGAACTTACGTCGTTATGTGCTCTAGGG |
| | CCATCACAC (SEQ ID NO: 36) |
| mm-HR4X-Ager-1280 | GATGGAATGTGGGGGAGCTTATACGTCGAGTTGAAC |
| | ATAAGTGCG (SEQ ID NO: 37) |
| mm-HL4X-Ager-1031 | TTAGTAGGCGAACTTACGTCGTTATGTCACCCACAG |
| | AGCCTTCAG (SEQ ID NO: 38) |
| mm-HR4X-Ager-1031 | CTAGCGTACCCAGCCCAGACTTATACGTCGAGTTGA |
| | ACATAAGTGCG (SEQ ID NO: 39) |
| mm-HL4X-Ager-771 | TTAGTAGGCGAACTTACGTCGTTATGTATTCCACCTT |
| | CAGGCTCAA (SEQ ID NO: 40) |
| mm-HR4X-Ager-771 | AGTCCCACCAGGAGCGACTTATACGTCGAGTTGAAC |
| | ATAAGTGCG (SEQ ID NO: 41) |
| mm-HL4X-Ager-652 | TTAGTAGGCGAACTTACGTCGTTATGTGCAGGAGAA |
| | GGTAGGATGG (SEQ ID NO: 42) |
| mm-HR4X-Ager-652 | GAAGGCCCAGGCTGAAACTTATACGTCGAGTTGAAC |
| | ATAAGTGCG (SEQ ID NO: 43) |
| mm-HL4X-Ager-496 | TTAGTAGGCGAACTTACGTCGTTATGTAAGGGTCCC |
| | TGCAGGGTAG (SEQ ID NO: 44) |
| mm-HR4X-Ager-496 | TTTCCCATCTAAGTGCCAGCTTATACGTCGAGTTGA |
| | ACATAAGTGCG (SEQ ID NO: 45) |
| mm-HL4X-Ager-436 | TTAGTAGGCGAACTTACGTCGTTATGTGGCTGTGAG |
| | TTCAGAGGC (SEQ ID NO: 46) |
| mm-HR4X-Ager-436 | TCCCCACCTTATTAGGGACACTTATACGTCGAGTTG |
| | AACATAAGTGCG (SEQ ID NO: 47) |
| mm-HL4X-Ager-350 | TTAGTAGGCGAACTTACGTCGTTATGTTCCCTCGCCT |
| | GTTAGTTG (SEQ ID NO: 48) |
| mm-HR4X-Ager-350 | CGGTAGTTGGACTTGACCTCCTTATACGTCGAGTTG |
| | AACATAAGTGCG (SEQ ID NO: 49) |
| mm-HL4X-Ager-1211 | TTAGTAGGCGAACTTACGTCGTTATGTCCGCTTCCTC |
| | TGACTGATT (SEQ ID NO: 50) |
| mm-HR4X-Ager-1211 | GGCACCATTCTCTGGCATCTTATACGTCGAGTTGAA |
| | CATAAGTGCG (SEQ ID NO: 51) |
| mm-HL4X-Ager-976 | TTAGTAGGCGAACTTACGTCGTTATGTGACAGGAGG |
| | GCTTTCCTG (SEQ ID NO: 52) |
| mm-HR4X-Ager-976 | GGTTTCTGTGACCCTGATGCTTATACGTCGAGTTGA |
| | ACATAAGTGCG (SEQ ID NO: 53) |
| mm-HL3X-Ftl1-808 | TATTCGTTCGAACTTACGTCGTTATGTCCTAGTCGTG |
| | CTTGAGAGTGA (SEQ ID NO: 54) |
| mm-HR3X-Ftl1-808 | CCTTGGAAGGTACAGAGGCCTTATACGTCGAGTTGA |
| | CCGACGTATTG (SEQ ID NO: 55) |
| mm-HL3X-Ftl1-515 | TATTCGTTCGAACTTACGTCGTTATGTTGAGATGGCT |
| | TCTGCACAT (SEQ ID NO: 56) |
| mm-HR3X-Ftl1-515 | TGGGTTTTACCCCATTCATCTTATACGTCGAGTTGAC |
| | CGACGTATTG (SEQ ID NO: 57) |
| mm-HL3X-Ftl1-152 | TATTCGTTCGAACTTACGTCGTTATGTTGTAAAGGC |
| | GGCTGGAAG (SEQ ID NO: 58) |
| mm-HR3X-Ftl1-152 | AGGCTGCGACTGGAGAGAC:TTATACGTCGAGTTGA |
| | CCGACGTATTG (SEQ ID NO: 59) |
| mm-HL3X-Ftl1-204 | TATTCGTTCGAACTTACGTCGTTATG:TAGGAGCTGA |
| | AGGCAG CG (SEQ ID NO: 60) |
| mm-HR3X-Ftl1-204 | GAC GGT GCA GAC TGG |
| | TCC: TTATACGTCGAGTTGACCGACGTATTG |
| | (SEQ ID NO: 61) |
| mm-HL3X-Ftl1-152 | TATTCGTTCGAACTTACGTCGTTATG: T TGT AAA |
| | GGC GGC TGG AAG (SEQ ID NO: 58) |
| mm-HR3X-Ftl1-152 | AGGCTGCGACTGGAGAGA |
| | C: TTATACGTCGAGTTGACCGACGTATTG |
| | (SEQ ID NO: 59) |
| mm-HL3X-Ftl1-204 | TATTCGTTCGAACTTACGTCGTTATG: T AGG AGC |
| | TGA AGG CAG CG (SEQ ID NO: 60) |
| mm-HR3X-Ftl1-204 | GAC GGT GCA GAC TGG |
| | TCC: TTATACGTCGAGTTGACCGACGTATTG |
| | (SEQ ID NO: 61) |
| mm-HL3X-Ftl1-808 | TATTCGTTCGAACTTACGTCGTTATG: TCCTAGTCGT |
| | GCTTGAGAGTGA (SEQ ID NO: 54) |
| mm-HR3X-Ftl1-808 | CCTTGGAAGGTACAGAGGCC: TTATACGTCGAGTTG |
| | ACCGACGTATTG (SEQ ID NO: 55) |
| mm-HL3X-Ftl1-515 | TATTCGTTCGAACTTACGTCGTTATG: TTGAGATGGC |
| | TTCTGCACAT (SEQ ID NO: 56) |
| mm-HR3X-Ftl1-515 | TGGGTTTTACCCCATTCATC: TTATACGTCGAGTTGA |
| | CCGACGTATTG (SEQ ID NO: 57) |
| mm-HL3X-Ftl1-391 | TATTCGTTCGAACTTACGTCGTTATGTCCAGAGCCA |
| | CGTCATCC (SEQ ID NO: 62) |
| mm-HR3X-Ftl1-391 | AAGAAGTGGCCTACGCCCTTATACGTCGAGTTGACC |
| | GACGTATTG (SEQ ID NO: 63) |
| mm-HL5X-Scgb1a1- | TAGCGCTAACAACTTACGTCGTTATGTCTTCAGCTG |
| 229 | GGTGCCC (SEQ ID NO: 64) |
| mm-HR5X-Scgb1a1- | GGGAGGGTATCCACCAGTCTTATACGTCGAGTTGAA |
| 229 | CGTCGTA ACA (SEQ ID NO: 65) |
| mm-HL5X-Scgb1a1- | TAGCGCTAACAACTTACGTCGTTATGTCTGAAATCC |
| 367 | AGTGAGCTTCAG (SEQ ID NO: 66) |
| mm-HR5X-Scgb1a1- | TGACAAGGCTTTAGCAGTAGAATATCTTATACGTCG |
| 367 | AGTTGAACGTCGTAACA (SEQ ID NO: 67) |
| mm-HL2X-Sftpc-783 | TCGTACGTCTAACTTACGTCGTTATGTTTATTCTTTT |
| | GTGATAGGATCCC (SEQ ID NO: 68) |
| mm-HR2X-Sftpc-783 | TTGTTTTCCAATCAGGCTGCTT ATA |
| | CGTCGAGTTGAAGAACAACCT G (SEQ ID NO: 69) |
| mm-HL2X-Sftpc-646 | TCG TAC GTC TAA |
| | CTTACGTCGTTATGTGCGGTTTCTACCGACC |
| | (SEQ ID NO: 70) |
| mm-HR2X-Sftpc-646 | GGTCTTTCCTGTCCCGCTT ATA CGT CGA GTT GAA |
| | GAA CAA CCTG (SEQ ID NO: 71) |
| mm-HL6X-Actb-610 | TAGGTCAGGAAACTTACGTCGTTATGTCGTAGATGG |
| | GCACAGTGTG (SEQ ID NO: 72) |
| mm-HR6X-Actb-610 | GTG AGGGAGAGCATAGCC |
| | CTTATACGTCGAGTTGAATAGCCAGGTT |
| | (SEQ ID NO: 73) |
| mm-HL6X-Actb-1111 | TAGGTCAGGAAACTTACGTCGTTATGTCAGGAGGAG |
| | CAATGA TCTTG (SEQ ID NO: 74) |
| mm-HR6X-Actb-1111 | TCCACACAGAGTACTTGCGC: TTATACGTCGAGTTGA |
| | ATAGCCAGGTT (SEQ ID NO: 75) |
| mm-HL6X-Actb-1465 | TAGGTCAGGAAACTTACGTCGTTATGTCCAACCAAC |
| | TGCTGTCG (SEQ ID NO: 76) |
| mm-HR6X-Actb-1465 | AACTTTGGGGGATGTTTGCTTATACGTCGAGTTGAA |
| | TAGCCAGGTT (SEQ ID NO: 77) |
| mm-HL6X-Actb-1771 | TAGGTCAGGAAACTTACGTCGTTATGTGGGCCATTC |
| | AGAAATTAA AA (SEQ ID NO: 78) |
| mm-HR6X-Actb-1771 | AAAAGGGAGGCCTCAGACCTTATACGTCGAGTTGA |
| | ATAGCCAGGTT (SEQ ID NO: 79) |
| mm-HL11X-Gapdh- | TGTTCGTGTTAACTTACGTCGTTATGACTGGAACAT |
| 360 | GTAGACCATG (SEQ ID NO: 80) |
| mm-HR11X-Gapdh- | TTTGCCGTGAGTGGAGTCATTTATACGTCGAGTTGA |
| 360 | ATCTTGATTCGGT (SEQ ID NO: 81) |
| mm-HL11X-Gapdh- | TGTTCGTGTTAACTTACGTCGTTATGATTTCTCGTGG |
| 632 | TTCACACCC (SEQ ID NO: 82) |
| mm-HR11X-Gapdh- | AATCTTGAGTGAGTTGTCATTTATACGTCGAGTTGA |
| 632 | ATCTTGATTCGGT (SEQ ID NO: 83) |
| mm-HL11X-Gapdh- | TGTTCGTGTTAACTTACGTCGTTATGAGGAACACGG |
| 923 | AAGGCCATGC (SEQ ID NO: 84) |
| mm-HR11X-Gapdh- | ACGACGGACACATTGGGGGTTTATACGTCGAGTTGA |
| 923 | ATCTTGATTCGGT (SEQ ID NO: 85) |
| mm-HL11X-Gapdh- | TGTTCGTGTTAACTTACGTCGTTATGACCAGGAAAT |
| 1155 | GAGCTTGACA (SEQ ID NO: 86) |
| mm-HR11X-Gapdh- | GTAGCCGTATTCATTGTCATTTATACGTCGAGTTGA |
| 1155 | ATCTTGATTCGGT (SEQ ID NO: 87) |
| mm-HL11X-Gapdh- | TGTTCGTGTTAACTTACGTCGTTATGATTGATGGTAT |
| 1385 | TCAAGAGAG (SEQ ID NO: 88) |
| mm-HR11X-Gapdh- | TTGTGGGTGCAGCGAACTTTTTATACGTCGAGTTGA |
| 1385 | ATCTTGATTCGGT (SEQ ID NO: 89) |
| mm-HL7X-Xist-1447 | GTGTAATTGTCACTTACGTCGTTATGTTAGACACATT |
| | CAAGAGCAT (SEQ ID NO: 90) |
| mm-HR7X-Xist-1447 | TCACATGACTTCCGCCATCTTATACGTCGAGTTGAT |
| | ATTCCTCAGG (SEQ ID NO: 91) |
| mm-HL7X-Xist-2130 | GTGTAATTGTCACTTACGTCGTTATGTTGAGATCAGT |
| | GCTGGCTAA (SEQ ID NO: 92) |
| mm-HR7X-Xist-2130 | CAGTAGGCTTAGAGAACCGCTTATACGTCGAGTTGA |
| | TATTCCTCAGG (SEQ ID NO: 93) |
| mm-HL7X-Xist-8362 | GTGTAATTGTCACTTACGTCGTTATGTAAGCTTCCTT |
| | CTCAGGAAG (SEQ ID NO: 94) |
| mm-HR7X-Xist-8362 | AAGAACGGAAAGAAAAGTGCTTATACGTCGAGTTG |
| | ATATTCCTCAGG (SEQ ID NO: 95) |
| mm-HL7X-Xist-10145 | GTGTAATTGTCACTTACGTCGTTATGTTGATCACGCT |
| | GAAGACCCA (SEQ ID NO: 96) |
| mm-HR7X-Xist-10145 | CTTTTGTGTGTTCACATTGCTTATACGTCGAGTTGAT |
| | ATTCCTCAGG (SEQ ID NO: 97) |
| mm-HL7X-Xist-16942 | GTGTAATTGTCACTTACGTCGTTATGTTTCGTTCCTC |
| | CAGTTTTCT (SEQ ID NO: 98) |
| mm-HR7X-Xist-16942 | CAAGTTCTTTCAATTTGGTCTTATACGTCGAGTTGAT |
| | ATTCCTCAGG (SEQ ID NO: 99) |
| mm-HL12X-Lyz2-920 | TCAAGTATGGTACTTACGTCGTTATGTCCTGTGCCCT |
| | CAGAAACCT (SEQ ID NO: 100) |
| mm-HR12X-Lyz2-920 | TCAGTTCTCATCCACTGAGACTTATACGTCGAGTTG |
| | AGATGTATCCTG (SEQ ID NO: 101) |
| mm-HL12X-Lyz2-791 | TCAAGTATGGTACTTACGTCGTTATGTTGTATTTTTA |
| | TTCACACAG (SEQ ID NO: 102) |
| mm-HR12X-Lyz2-791 | TGGTCTCCTATAAGCAGTTCTTATACGTCGAGTTGA |
| | GATGTATCCTG (SEQ ID NO: 103) |
| mm-HL12X-Lyz2-607 | TCAAGTATGGTACTTACGTCGTTATGTGAAGCTTGT |
| | GAGGAGGAAG (SEQ ID NO: 104) |
| mm-HR12X-Lyz2-607 | TTGCACAACACAGCATCAGCTTATACGTCGAGTTGA |
| | GATGTATCCTG (SEQ ID NO: 105) |
| mm-HL12X-Lyz2-422 | TCAAGTATGGTACTTACGTCGTTATGTCGGTTTTGAC |
| | AGTGTGCTC (SEQ ID NO: 106) |
| mm-HR12X-Lyz2-422 | CGAATATACTGGGACAGATCTTATACGTCGAGTTGA |
| | GATGTATCCTG (SEQ ID NO: 107) |
| mm-HL12X-Lyz2-246 | TCAAGTATGGTACTTACGTCGTTATGTATTGATCTGA |
| | AATATCCCA (SEQ ID NO: 108) |
| mm-HR12X-Lyz2-246 | ATCATTACACCAGTATCGGCTTATACGTCGAGTTGA |
| | GATGTATCCTG (SEQ ID NO: 109) |
| mm-HL12X-Lyz2-67 | TCAAGTATGGTACTTACGTCGTTATGTTGGCCTGAG |
| | CAGTGACAGA (SEQ ID NO: 110) |
| mm-HR12X-Lyz2-67 | ACTCACAACGTTCATAGACCTTATACGTCGAGTTGA |
| | GATGTATCCTG (SEQ ID NO: 111) |

Blocking oligonucleotides - Scgb1a1

| | |
|---|---|
| TRN10 + HLR + 10- | GGTCTCTTGTGGGAGGGTATCCACCAGTCTCTT |
| Scgb1a1-248 | CAGCTGGGTGCCCGCATTTTGCA (SEQ ID NO: 112) |
| Control | TATCGTGTAATCAGTCTCCCAAAGACTCGGACAACA |
| Scrambled oligo | TAACGACGTAAGTTAGACGTACGA (SEQ ID NO: 113) |

Short imager oligo

| | |
|---|---|
| ImO-Y1-A488-2 | /5Alex488N/TGAGCTAGAAT (SEQ ID NO: 114) |

Mouse Axin2 - PLISH probes

| | |
|---|---|
| mm-HL3X-Axin2-2956 | TATTCGTTCGAACTTACGTCGTTATG: ATTTCGTGGC |
| | TGTTGCGTA (SEQ ID NO: 115) |
| mm-HR3X-Axin2-2956 | TTTCATTTTCCTTCAGAAT: TTATACGTCGAGTTGACC |
| | GACGTATTG (SEQ ID NO: 116) |
| mm-HL3X-Axin2-2412 | TATTCGTTCGAACTTACGTCGTTATG: AGCTGTGCCA |
| | AAGTGTTGG (SEQ ID NO: 117) |
| mm-HR3X-Axin2-2412 | CCTGCGGCAGGCTTCCTCT: TTATACGTCGAGTTGAC |
| | CGACGTATTG (SEQ ID NO: 118) |
| mm-HL3X-Axin2-1878 | TATTCGTTCGAACTTACGTCGTTATG: AGTGGTGGTG |
| | AACGTGCT (SEQ ID NO: 119) |
| mm-HR3X-Axin2-1878 | ACAGCGTGGTGGTGGATGT: TTATACGTCGAGTTGAC |
| | CGACGTATTG (SEQ ID NO: 120) |
| mm-HL3X-Axin2-347 | TATTCGTTCGAACTTACGTCGTTATG: AACACGGCGC |
| | TACTCATGGT (SEQ ID NO: 121) |
| mm-HR3X-Axin2-347 | GATCTGGAAGGAGAGTCACT: TTATACGTCGAGTTG |
| | ACCGACGTATTG (SEQ ID NO: 122) |

Mouse Axin2 - HCR probes

| | |
|---|---|
| HCR1-H1-A488 | GAAGCGAATATGGTGAGAGTTGGAGGTAGGTTGAGGCAC |
| | ATTTACAGACCTCAACCTACCTCCAACTCTCAC |
| | (SEQ ID NO: 123) |
| HCR1-H2-A488 | CCTCAACCTACCTCCAACTCTCACCATATTCGCTTCGTGAG |
| | AGTTGGAGGTAGGTTGAGGTCTGTAAATGTG |
| | (SEQ ID NO: 124) |
| HCR1-Axin2- | CCTCAACCTACCTCCAACTCTCACCATATTCGCTTCTAAAA |
| 621-A488 | CGTATCCACACATTTCTCCCTCTCCAGGAAAGTCCGGAAG |
| | AGGTATGCACATTTTCACATTTACAGACCTCAACCTACCTC |
| | CAACTCTCAC (SEQ ID NO: 125) |
| HCR2-H1-A647 | GGCGGTTTACTGGATGATTGATGAGGATTTACGAGGAGCT |
| | CAGTCCATCCTCGTAAATCCTCATCAATCATC |
| | (SEQ ID NO: 126) |
| HCR2-H2-A647 | /5Cy5/CCTCGTAAATCCTCATCAATCATCCAGTAAACCGCCG |
| | ATGATTGATGAGGATTTACGAGGATGGACTGAGCT |
| | (SEQ ID NO: 127) |
| HCR2-Axin2- | CCTCGTAAATCCTCATCAATCATCCAGTAAACCGCCAAAA |
| 1159-A647 | AAACCTACGTGATAAGGATTGACTGGGTCGCTTCTCTTGA |
| | AGGACCTGAATAAAAAAGCTCAGTCCATCCTCGTAAATCC |
| | TCATCAATCATCC (SEQ ID NO: 128) |

Human PLISH probes

| | |
|---|---|
| HL4X-MLLT10-4613 | TTAGTAGGCGAACTTACGTCGTTATGTGTCACATCA |
| | AAAGCGTGG (SEQ ID NO: 129) |
| HR4X-MLLT10-4613 | GGGGAATCACTCTGCGGTACTTATACGTCGAGTTGA |
| | ACATAAGTGCG (SEQ ID NO: 130) |
| HL4X-MLLT10-4532 | TTAGTAGGCGAACTTACGTCGTTATGTTTTAATGGTC |
| | AGTAATGGGAAGTG (SEQ ID NO: 131) |
| HR4X-MLLT10-4532 | TTTCAGGAACTCTAGTGGTGAGCTTATACGTCGAGT |
| | TGAACATAAGTGCG (SEQ ID NO: 132) |
| HL5X-MLLT10-4613 | TAGCGCTAACAACTTACGTCGTTATGTGTCACATCA |
| | AAAGCGTGG (SEQ ID NO: 133) |
| HL5X-MLLT10-4418 | TAGCGCTAACAACTTACGTCGTTATGTGAGCTATCT |
| | ACAGTGGCACAGA (SEQ ID NO: 134) |
| HL5X-MLLT10-4277 | TAGCGCTAACAACTTACGTCGTTATGTTGATCAAGG |
| | GCAAACCC (SEQ ID NO: 135) |
| HL5X-MLLT10-3629 | TAGCGCTAACAACTTACGTCGTTATGTTAC AAGAAT |
| | GTCCTTGCAGCA (SEQ ID NO: 136) |
| HR5X-MLLT10-4613 | GGGGAATCACTCTGCGGTACTTATACGTCGAGTTGA |
| | ACGTCGTAACA (SEQ ID NO: 137) |
| HR5X-MLLT10-4418 | AAATACCCAGGTAATAGTTTTGCCTTATACGTCGAG |
| | TTGAACGTCGTAACA (SEQ ID NO: 138) |
| HR5X-MLLT10-4277 | GCAGCACAATGGAGGCACTTATACGTCGAGTTGAAC |
| | GTCGTAACA (SEQ ID NO: 139) |
| HR5X-MLLT10-3629 | GCAACAAGAAAACTAATCAAAGCCTTATACGTCGA |
| | GTTGAACGTCGTAACA (SEQ ID NO: 140) |
| HL5X-MLLT10-4532 | TAGCGCTAACAACTTACGTCGTTATGTTTTAATGGTC |
| | AGTAATGGGAAGTG (SEQ ID NO: 141) |
| HL5X-MLLT10-4338 | TAGCGCTAACAACTTACGTCGTTATGTGCTCGGAGG |
| | ACTGACTGT (SEQ ID NO: 142) |
| HL5X-MLLT10-4177 | TAGCGCTAACAACTTACGTCGTTATGTCACTTAACT |
| | CAAGATTTCTTCAAAA (SEQ ID NO: 143) |
| HL5X-MLLT10-3579 | TAGCGCTAACAACTTACGTCGTTATGTTGTTGCGTTT |
| | CTTCATAGCTG (SEQ ID NO: 144) |
| HL5X-MLLT10-2221 | TAGCGCTAACAACTTACGTCGTTATGTCCTACTTGTT |
| | GCAAATGCC (SEQ ID NO: 145) |
| HL5X-MLLT10-1312 | TAGCGCTAACAACTTACGTCGTTATGTGAAGATTTC |
| | TTCCCTTTGCC (SEQ ID NO: 146) |
| HR5X-MLLT10-1312 | CTTTGACCTGAGCTGTGAGCTTATACGTCGAGTTGA |
| | ACGTCGTAACA (SEQ ID NO: 147) |
| HR5X-MLLT10-4532 | TTTCAGGAACTCTAGTGGTGAGCTTATACGTCGAGT |
| | TGAACGTCGTAACA (SEQ ID NO: 148) |
| HR5X-MLLT10-4338 | GTGACGGGATGATTCCTGCTTATACGTCGAGTTGAA |
| | CGTCGTAACA (SEQ ID NO: 149) |
| HR5X-MLLT10-4177 | CCCCAGCAGCCTCAGAACTTATACGTCGAGTTGAAC |
| | GTCGTAACA (SEQ ID NO: 150) |
| HR5X-MLLT10-3579 | CCTTTGTTGTGCATTGAGTTTCTTATACGTCGAGTTG |
| | AACGTCGTAACA (SEQ ID NO: 151) |
| HR5X-MLLT10-2221 | AGCTGAGGGAGAGAGCGCTTATACGTCGAGTTGAA |
| | CGTCGTAACA (SEQ ID NO: 152) |
| HL2X-MLLT10-4613 | TCGTACGTCTAACTTACGTCGTTATGTGTCACATCAA |
| | AAGCGTGG (SEQ ID NO: 153) |
| HL2X-MLLT10-4418 | TCGTACGTCTAACTTACGTCGTTATGTGAGCTATCTA |
| | CAGTGGCACAGA (SEQ ID NO: 154) |
| HL2X-MLLT10-4277 | TCGTACGTCTAACTTACGTCGTTATGTTGATCAAGG |
| | GCAAACCC (SEQ ID NO: 155) |
| HL2X-MLLT10-3629 | TCGTACGTCTAACTTACGTCGTTATGTTACAAGAAT |
| | GTCCTTGCAGCA (SEQ ID NO: 156) |
| HL2X-MLLT10-2559 | TCGTACGTCTAACTTACGTCGTTATGTCCAAAAGCT |
| | GTTCTATGCTG (SEQ ID NO: 157) |
| HL2X-MLLT10-1475 | TCGTACGTCTAACTTACGTCGTTATGTCAGGAAATC |
| | CTGGGGAGAC (SEQ ID NO: 158) |
| HR2X-MLLT10-4613 | GGGGAATCACTCTGCGGTACTTATACGTCGAGTTGA |
| | AGAACAACCTG (SEQ ID NO: 159) |
| HR2X-MLLT10-4418 | AAATACCCAGGTAATAGTTTTGCCTTATACGTCGAG |
| | TTGAAGAACAACCTG (SEQ ID NO: 160) |
| HR2X-MLLT10-4277 | GCAGCACAATGGAGGCACTTATACGTCGAGTTGAA |
| | GAACAACCTG (SEQ ID NO: 161) |
| HR2X-MLLT10-3629 | GCAACAAGAAAACTAATCAAAGCCTTATACGTCGA |
| | GTTGAAGAACAACCTG (SEQ ID NO: 162) |
| HR2X-MLLT10-2559 | GTCCTTCACTCCACTGCCTCTTATACGTCGAGTTGAA |
| | GAACAACCTG (SEQ ID NO: 163) |
| HR2X-MLLT10-1475 | CGCAGATCTGAGTCTGTAAAGCTTATACGTCGAGTT |
| | GAAGAACAACCTG (SEQ ID NO: 164) |
| HL6X-ETV5-1840 | TAGGTCAGGAAACTTACGTCGTTATGTTGTTTGCCT |
| | GAATGGGAA (SEQ ID NO: 165) |
| HR6X-ETV5-1840 | CACAAACAAAACCACTGCCCTTATACGTCGAGTTGA |
| | ATAGCCAGGTT (SEQ ID NO: 166) |
| HL6X-ETV5-1911 | TAGGTCAGGAAACTTACGTCGTTATGTTGGGTTCTC |
| | CAGATAATACTCAA (SEQ ID NO: 167) |
| HR6X-ETV5-1911 | GTGCCAATCCAGAGACAGCTTATACGTCGAGTTGAA |
| | TAGCCAGGTT (SEQ ID NO: 168) |
| HL2X-ETV5-1424 | TCGTACGTCTAACTTACGTCGTTATGTCGACCTGTCC |
| | AGGCAAT (SEQ ID NO: 169) |
| HR2X-ETV5-1424 | ATCAGCTTGAACTCCATGCCTTATACGTCGAGTTGA |
| | AGAACAACCTG (SEQ ID NO: 170) |
| HL2X-ETV5-1709 | TCGTACGTCTAACTTACGTCGTTATGTTCAAAGTGG |
| | GTCAGCGG (SEQ ID NO: 171) |
| HR2X-ETV5-1709 | GAGGTAAGCGGGGCTGTCTTATACGTCGAGTTGAAG |
| | AACAACCTG (SEQ ID NO: 172) |
| HL2X-ETV5-3344 | TCGTACGTCTAACTTACGTCGTTATGTGCCTCCTTCA |
| | CATACGAGG (SEQ ID NO: 173) |
| HR2X-ETV5-3344 | CACACTCACGGAGTCAGCACTTATACGTCGAGTTGA |
| | AGAACAACCTG (SEQ ID NO: 174) |
| HL2X-ETV5-3453 | TCGTACGTCTAACTTACGTCGTTATGTCTTCACAAAT |
| | GTGCTGTTGG (SEQ ID NO: 175) |
| HR2X-ETV5-3453 | CCCTTATCCCTGCGAACCTTATACGTCGAGTTGAAG |
| | AACAACCTG (SEQ ID NO: 176) |
| HL2X-ETV5-1821 | TCGTACGTCTAACTTACGTCGTTATGTTGTTTGCCTG |
| | AATGGGAA (SEQ ID NO: 177) |
| HR2X-ETV5-1840 | CACAAACAAAACCACTGCCCTTATACGTCGAGTTGA |
| | AGAACAACCTG (SEQ ID NO: 178) |
| HL5X-ETV5-3453 | TAGCGCTAACAACTTACGTCGTTATGTCTTCACAAA |
| | TGTGCTGTTGG (SEQ ID NO: 179) |
| HR5X-ETV5-3453 | CCCTTATCCCTGCGAACCTTATACGTCGAGTTGAAC |
| | GTCGTAACA (SEQ ID NO: 180) |
| HL5X-ETV5-222 | TAGCGCTAACAACTTACGTCGTTATGTTTGAGAGGT |
| | TTCAGCATTGAG (SEQ ID NO: 181) |
| HR5X-ETV5-222 | GCTTTCAGCGTCTCTAATACCACTTATACGTCGAGTT |
| | GAACGTCGTAACA (SEQ ID NO: 182) |
| HL5X-ETV5-1132 | TAGCGCTAACAACTTACGTCGTTATGTCCTGCTTGAT |
| | TCCCATTG (SEQ ID NO: 183) |
| HR5X-ETV5-1132 | ACGCAGTAATCC CGAGGCTTATACGTCGAGTTGAAC |
| | GTCGTAACA (SEQ ID NO: 184) |
| HL5X-ETV5-1991 | TAGCGCTAACAACTTACGTCGTTATGTTTTGGTGGTT |
| | TTCTGCCC (SEQ ID NO: 185) |
| HR5X-ETV5-1991 | AAGAGTTGAGGCACTGGCCTTATACGTCGAGTTGAA |
| | CGTCGTAACA (SEQ ID NO: 186) |
| HL5X-ETV5-2091 | TAGCGCTAACAACTTACGTCGTTATGTGAACAAGAT |
| | ATTGCTTTGCATG (SEQ ID NO: 187) |
| HR5X-ETV5-2091 | CCGTTTTGCGGGTACTAACCTTATACGTCGAGTTGA |
| | ACGTCGTAACA (SEQ ID NO: 188) |
| HL5X-ETV5-2423 | TAGCGCTAACAACTTACGTCGTTATGTACTTGGCGA |
| | CCACATGG (SEQ ID NO: 189) |
| HR5X-ETV5-2423 | AGAGAAAGGGGGCTTGCTCTTATACGTCGAGTTGAA |
| | CGTCGTAACA (SEQ ID NO: 190) |
| HL5X-ETV5-2508 | TAGCGCTAACAACTTACGTCGTTATGTGTCAAAAGT |
| | GTTAATCGCCC (SEQ ID NO: 191) |
| HR5X-ETV5-2508 | CAAATCAGGGCAAAACAAAATACTTATACGTCGAG |
| | TTGAACGTCGTAACA (SEQ ID NO: 192) |
| HL5X-ETV5-2672 | TAGCGCTAACAACTTACGTCGTTATGTGGCTAAAGG |
| | ACACAGTGCA (SEQ ID NO: 193) |
| HR5X-ETV5-2672 | CATAAGTTGGCACGGCCCTTATACGTCGAGTTGAAC |
| | GTCGTAACA (SEQ ID NO: 194) |
| HL5X-ETV5-2837 | TAGCGCTAACAACTTACGTCGTTATGTGCCCTGCAC |
| | ACTTCAAA (SEQ ID NO: 195) |
| HR5X-ETV5-2837 | GAATCTAAGCTTGTGTCCAGGCTTATACGTCGAGTT |
| | GAACGTCGTAACA (SEQ ID NO: 196) |
| HL5X-ETV5-1887 | TAGCGCTAACAACTTACGTCGTTATGTTGGGTTCTCC |
| | AGATAATACTCAA (SEQ ID NO: 197) |
| HR5X-ETV5-1911 | GTGCCAATCCAGAGACAGCTTATACGTCGAGTTGAA |
| | CGTCGTAACA (SEQ ID NO: 198) |
| HL3X-CASP9-1542 | TATTCGTTCGAACTTACGTCGTTATGTCTTCCTCCAC |
| | TGTTCAGCA (SEQ ID NO: 199) |
| HR3X-CASP9-1542 | ACGGCATTCATCTGTCCCTTATACGTCGAGTTGACC |
| | GACGTATTG (SEQ ID NO: 200) |
| HL3X-CASP9-972 | TATTCGTTCGAACTTACGTCGTTATGTTTCTGCTCCC |
| | CACCACAG (SEQ ID NO: 201) |
| HR3X-CASP9-972 | CCACCTCAAACCCATGGTCTTATACGTCGAGTTGAC |
| | CGACGTATTG (SEQ ID NO: 202) |
| HL5X-CASP9-1850 | TAGCGCTAACAACTTACGTCGTTATGTACAGACCAG |
| | TGGTTGTCAGG (SEQ ID NO: 203) |
| HR5X-CASP9-1850 | TTGTGTGTAGCCAAAAATCCCTTATACGTCGAGTTG |
| | AACGTCGTAACA (SEQ ID NO: 204) |
| HL5X-CASP9-1789 | TAGCGCTAACAACTTACGTCGTTATGTGAGAACCTT |
| | TTTGTTTGGCA (SEQ ID NO: 205) |
| HR5X-CASP9-1789 | CAGGACATATTTGGCAACACCTTATACGTCGAGTTG |
| | AACGTCGTAACA (SEQ ID NO: 206) |
| HL5X-CASP9-1695 | TAGCGCTAACAACTTACGTCGTTATGTCACAGCAAA |
| | GGGTGACCT (SEQ ID NO: 207) |
| HR5X-CASP9-1695 | CCACAATGTACAGGACAGCCTTATACGTCGAGTTGA |
| | ACGTCGTAACA (SEQ ID NO: 208) |
| HL5X-CASP9-1292 | TAGCGCTAACAACTTACGTCGTTATGTGTTTATAAA |
| | TCCCTTTCACCGA (SEQ ID NO: 209) |
| HR5X-CASP9-1292 | GAAATTAAAGCAACCAGGCATCTTATACGTCGAGTT |
| | GAACGTCGTAACA (SEQ ID NO: 210) |
| HL5X-CASP9-1211 | TAGCGCTAACAACTTACGTCGTTATGTCAAAGATGT |
| | CGTCCAGGGT (SEQ ID NO: 211) |
| HR5X-CASP9-1211 | TTCAGAGTGAGCCCACTGCTTATACGTCGAGTTGAA |
| | CGTCGTAACA (SEQ ID NO: 212) |
| HL5X-CASP9-1096 | TAGCGCTAACAACTTACGTCGTTATGTAGATATGGC |
| | GTCCAGCTGG (SEQ ID NO: 213) |
| HR5X-CASP9-1096 | CACTGGGTGTGGGCAAACTTATACGTCGAGTTGAAC |
| | GTCGTAACA (SEQ ID NO: 214) |
| HL5X-CASP9-881 | TAGCGCTAACAACTTACGTCGTTATGTTCTCGACCG |
| | ACACAGGG (SEQ ID NO: 215) |
| HR5X-CASP9-881 | CCCATTGAAGATGTTCACAATCTTATACGTCGAGTT |
| | GAACGTCGTAACA (SEQ ID NO: 216) |
| HL5X-CASP9-701 | TAGCGCTAACAACTTACGTCGTTATGTCCACCATGA |
| | AATGCAGC (SEQ ID NO: 217) |
| HR5X-CASP9-701 | AGTCAGGTCGCCCTTCACCTTATACGTCGAGTTGAA |
| | CGTCGTAACA (SEQ ID NO: 218) |
| HL5X-CASP9-341 | TAGCGCTAACAACTTACGTCGTTATGTGGCCTGTGT |
| | CCTCTAAGCA (SEQ ID NO: 219) |
| HR5X-CASP9-341 | AAACGAAGCCAGCATGTCCTTATACGTCGAGTTGAA |
| | CGTCGTAACA (SEQ ID NO: 220) |
| HL5X-CASP9-284 | TAGCGCTAACAACTTACGTCGTTATGTCCAGATCTA |
| | TGATCAGCTGC (SEQ ID NO: 221) |
| HR5X-CASP9-284 | AGCCTGACTCCCTCGAGTCTTATACGTCGAGTTGAA |
| | CGTCGTAACA (SEQ ID NO: 222) |
| HL2X-CASP9-1602 | TCGTACGTCTAACTTACGTCGTTATGTAGCCCTGGA |
| | CCAGCCAC (SEQ ID NO: 223) |
| HR2X-CASP9-1602 | GGATCATGGGACACAAGTCACTTATACGTCGAGTTG |
| | AAGAACAACCTG (SEQ ID NO: 224) |
| HL2X-CASP9-1542 | TCGTACGTCTAACTTACGTCGTTATGTCTTCCTCCAC |
| | TGTTCAGCA (SEQ ID NO: 225) |
| HR2X-CASP9-1542 | ACGGCATTCATCTGTCCCTTATACGTCGAGTTGAAG |
| | AACAACCTG (SEQ ID NO: 226) |
| HL2X-CASP9-1499 | TCGTACGTCTAACTTACGTCGTTATGTAAGAGCCTG |
| | TCTGTCACTGG (SEQ ID NO: 227) |
| HR2X-CASP9-1499 | GTCGTCAATCTGGAAGCTGCTTATACGTCGAGTTGA |
| | AGAACAACCTG (SEQ ID NO: 228) |
| HL2X-CASP9-1417 | TCGTACGTCTAACTTACGTCGTTATGTCAGCCTCTTT |
| | CAGGCCTG (SEQ ID NO: 229) |
| HR2X-CASP9-1417 | TGCAGGAAAGTCCAGGCCTTATACGTCGAGTTGAAG |
| | AACAACCTG (SEQ ID NO: 230) |
| HL2X-CASP9-1024 | TCGTACGTCTAACTTACGTCGTTATGTGCCAGGGGA |
| | CTCGTCTT (SEQ ID NO: 231) |
| HR2X-CASP9-1024 | CATCTGGCTCGGGGTTACTTATACGTCGAGTTGAAG |
| | AACAACCTG (SEQ ID NO: 232) |
| HL2X-CASP9-972 | TCGTACGTCTAACTTACGTCGTTATGTTTCTGCTCCC |
| | CACCACAG (SEQ ID NO: 233) |
| HR2X-CASP9-972 | CCACCTCAAACCCATGGTCTTATACGTCGAGTTGAA |
| | GAACAACCTG (SEQ ID NO: 234) |
| HL2X-CASP9-543 | TCGTACGTCTAACTTACGTCGTTATGTGCATTTCCCC |
| | TCAAACTCT (SEQ ID NO: 235) |
| HR2X-CASP9-543 | GCTCAGGATGTAAGCCAAATCTTATACGTCGAGTTG |
| | AAGAACAACCTG (SEQ ID NO: 236) |
| HL2X-CASP9-475 | TCGTACGTCTAACTTACGTCGTTATGTGGGTGTTTCC |
| | GGTCTGAG (SEQ ID NO: 237) |
| HR2X-CASP9-475 | CAGAACCAATGTCCACTGGTCTTATACGTCGAGTTG |
| | AAGAACAACCTG (SEQ ID NO: 238) |
| HL2X-CASP9-395 | TCGTACGTCTAACTTACGTCGTTATGTTCGACAACTT |
| | TGCTGCTTG (SEQ ID NO: 239) |
| HR2X-CASP9-395 | GGTAAGGTTTTCTAGGGTTGGCTTATACGTCGAGTT |
| | GAAGAACAACCTG (SEQ ID NO: 240) |
| HL2X-CASP9-82 | TCGTACGTCTAACTTACGTCGTTATGTAAGACTCCA |
| | GGCCGCCT (SEQ ID NO: 241) |
| HR2X-CASP9-82 | CCATGGCGAGTAGCCAACTTATACGTCGAGTTGAAG |
| | AACAACCTG (SEQ ID NO: 242) |
| HL2X-GAPDH-1085 | TCGTACGTCTAACTTACGTCGTTATGTACTTCTCATG |
| | GTTCACACCCA (SEQ ID NO: 243) |
| HR2X-GAPDH-1107 | GATGATCTTGAGGCTGTTGTCATTATACGTCGAGTT |
| | GAAGAACAACCTG (SEQ ID NO: 244) |
| HL2X-GAPDH-1329 | TCGTACGTCTAACTTACGTCGTTATGGATGACCTTGC |
| | CCACAGCCT (SEQ ID NO: 245) |
| HR2X-GAPDH-1349 | AGCTTCCCGTTCAGCTCAGGTTATACGTCGAGTTGA |
| | AGAACAACCTG (SEQ ID NO: 246) |
| HL2X-GAPDH-1757 | TCGTACGTCTAACTTACGTCGTTATGGACTGAGTGT |
| | GGCAGGGACT (SEQ ID NO: 247) |
| HR2X-GAPDH-1777 | GGAGATTCAGTGTGGTGGGGTTATACGTCGAGTTGA |
| | AGAACAACCTG (SEQ ID NO: 248) |
| HL2X-GAPDH-1334 | TCGTACGTCTAACTTACGTCGTTATGTCAGGGATGA |
| | CCTTGCCC (SEQ ID NO: 249) |
| HR2X-GAPDH-1353 | AGTGAGCTTCCCGTTCAGCTTATACGTCGAGTTGAA |
| | GAACAACCTG (SEQ ID NO: 250) |
| HL5X-GAPDH-1755 | TAGCGCTAACAACTTACGTCGTTATGTGAGTGTGGC |
| | AGGGACTCC (SEQ ID NO: 251) |
| HL5X-GAPDH-1715 | TAGCGCTAACAACTTACGTCGTTATGTTCCTCTTGTG |
| | CTCTTGCTG (SEQ ID NO: 252) |
| HL5X-GAPDH-1634 | TAGCGCTAACAACTTACGTCGTTATGTGTTGCTGTA |
| | GCCAAATTCG (SEQ ID NO: 253) |
| HL5X-GAPDH-1500 | TAGCGCTAACAACTTACGTCGTTATGTCAGTGTAGC |
| | CCAGGATGC (SEQ ID NO: 254) |
| HL5X-GAPDH-1390 | TAGCGCTAACAACTTACGTCGTTATGTGACACGTTG |
| | GCAGTGGG (SEQ ID NO: 255) |
| HL5X-GAPDH-1311 | TAGCGCTAACAACTTACGTCGTTATGTTGGCAGCGC |
| | CAGTAGAG (SEQ ID NO: 256) |
| HL5X-GAPDH-1224 | TAGCGCTAACAACTTACGTCGTTATGTTCTGGGTGG |
| | CAGTGATG (SEQ ID NO: 257) |
| HL5X-GAPDH-984 | TAGCGCTAACAACTTACGTCGTTATGTCCATGGTGG |
| | TGAAGACG (SEQ ID NO: 258) |
| HL5X-GAPDH-903 | TAGCGCTAACAACTTACGTCGTTATGTCCTGGAAGA |
| | TGGTGATGG (SEQ ID NO: 259) |
| HR5X-GAPDH-1755 | ATTCAGTGTGGTGGGGGACTTATACGTCGAGTTGAA |
| | CGTCGTAACA (SEQ ID NO: 260) |
| HR5X-GAPDH-1715 | CAGCAGTGAGGGTCTCTCTCTTATACGTCGAGTTGA |
| | ACGTCGTAACA (SEQ ID NO: 261) |
| HR5X-GAPDH-1634 | CCATGAGGTCCACCACCCTTATACGTCGAGTTGAAC |
| | GTCGTAACA (SEQ ID NO: 262) |
| HR5X-GAPDH-1500 | AGAGGAGACCACCTGGTGCTTATACGTCGAGTTGAA |
| | CGTCGTAACA (SEQ ID NO: 263) |
| HR5X-GAPDH-1390 | GCAGGTCAGGTCCACCACTTATACGTCGAGTTGAAC |
| | GTCGTAACA (SEQ ID NO: 264) |
| HR5X-GAPDH-1311 | ATGACCTTGCCCACAGCCTTATACGTCGAGTTGAAC |
| | GTCGTAACA (SEQ ID NO: 265) |
| HR5X-GAPDH-1224 | GAGGGGCCATCCACAGTCTTATACGTCGAGTTGAAC |
| | GTCGTAACA (SEQ ID NO: 266) |
| HR5X-GAPDH-984 | AAATGAGCCCCAGCCTTCTTATACGTCGAGTTGAAC |
| | GTCGTAACA (SEQ ID NO: 267) |
| HR5X-GAPDH-903 | GATTTTGGAGGGATCTCGCTTATACGTCGAGTTGAA |
| | CGTCGTAACA (SEQ ID NO: 268) |
| HL5X1-GAPDH-1757 | TAGCGCTAACATTAAGACGCTTGACTGAGTGTGGCA |
| | GGGACT (SEQ ID NO: 269) |
| HR5X1-GAPDH-1777 | GGAGATTCAGTGTGGTGGGGAAATATGACAGAACA |
| | CGTCGTAACA (SEQ ID NO: 270) |
| HL5X-GAPDH-1757 | TAGCGCTAACAACTTACGTCGTTATGGACTGAGTGT |
| | GGCAGGGACT (SEQ ID NO: 271) |
| HR5X-GAPDH-1777 | GGAGATTCAGTGTGGTGGGGTTATACGTCGAGTTGA |
| | ACGTCGTAACA (SEQ ID NO: 272) |
| HL4X-GAPDH-1084 | TTAGTAGGCGAACTTACGTCGTTATGACTTCTCATG |
| | GTTCACACCCA (SEQ ID NO: 273) |
| HR4X-GAPDH-1107 | GATGATCTTGAGGCTGTTGTCATTTATACGTCGAGTT |
| | GAACATAAGTGCG (SEQ ID NO: 274) |
| HL4X-GAPDH-1757 | TTAGTAGGCGAACTTACGTCGTTATGGACTGAGTGT |
| | GGCAGGGACT (SEQ ID NO: 275) |
| HR4X-GAPDH-1777 | GGAGATTCAGTGTGGTGGGGTTATACGTCGAGTTGA |
| | ACATAAGTGCG (SEQ ID NO: 276) |
| HL3X1-GAPDH-1757 | TATTCGTTCGATTAAGACGCTTGACTGAGTGTGGCA |
| | GGGACT (SEQ ID NO: 277) |
| HR3X1-GAPDH-1777 | GGAGATTCAGTGTGGTGGGGAAATATGACAGAACC |
| | CGACGTATTG (SEQ ID NO: 278) |
| HL3X-GAPDH-1757 | TATTCGTTCGAACTTACGTCGTTATGGACTGAGTGT |
| | GGCAGGGACT (SEQ ID NO: 279) |
| HR3X-GAPDH-1777 | GGAGATTCAGTGTGGTGGGGTTATACGTCGAGTTGA |
| | CCGACGTATTG (SEQ ID NO: 280) |
| HL5X-ACTB-1652 | TAGCGCTAACAACTTACGTCGTTATGTGGGCCATTC |
| | TCCTTAGAGA (SEQ ID NO: 281) |
| HL5X-ACTB-1490 | TAGCGCTAACAACTTACGTCGTTATGTGCTGTCACC |
| | TTCACCGTT (SEQ ID NO: 282) |
| HL5X-ACTB-1389 | TAGCGCTAACAACTTACGTCGTTATGTCATCTTGTTT |
| | TCTGCGCAA (SEQ ID NO: 283) |
| HL5X-ACTB-1272 | TAGCGCTAACAACTTACGTCGTTATGTGCTTGCTGA |
| | TCCACATCTG (SEQ ID NO: 284) |
| HL5X-ACTB-1176 | TAGCGCTAACAACTTACGTCGTTATGTTGATCTTCAT |
| | TGTGCTGGG (SEQ ID NO: 285) |
| HL5X-ACTB-908 | TAGCGCTAACAACTTACGTCGTTATGTCTTCTCCAG |
| | GGAGGAGCT (SEQ ID NO: 286) |
| HL5X-ACTB-834 | TAGCGCTAACAACTTACGTCGTTATGTCCTTAATGTC |
| | ACGCACGAT (SEQ ID NO: 287) |
| HL5X-ACTB-603 | TAGCGCTAACAACTTACGTCGTTATGTGGATAGCAA |
| | CGTACATGGC (SEQ ID NO: 288) |
| HL5X-ACTB-363 | TAGCGCTAACAACTTACGTCGTTATGTCGTCGCCCA |
| | CATAGGAAT (SEQ ID NO: 289) |
| HR5X-ACTB-1652 | GTGTGGACTTGGGAGAGGACTTATACGTCGAGTTGA |
| | ACGTCGTAACA (SEQ ID NO: 290) |
| HR5X-ACTB-1490 | ATGCTCGCTCCAACCGACTTATACGTCGAGTTGAAC |
| | GTCGTAACA (SEQ ID NO: 291) |
| HR5X-ACTB-1389 | CAAATAAAGCCATGCCAATCTTATACGTCGAGTTGA |
| | ACGTCGTAACA (SEQ ID NO: 292) |
| HR5X-ACTB-1272 | GCCGGACTCGTCATACTCCTTATACGTCGAGTTGAA |
| | CGTCGTAACA (SEQ ID NO: 293) |
| HR5X-ACTB-1176 | GCTCAGGAGGAGCAATGATCTTATACGTCGAGTTGA |
| | ACGTCGTAACA (SEQ ID NO: 294) |
| HR5X-ACTB-908 | CGTCAGGCAGCTCGTAGCTTATACGTCGAGTTGAAC |
| | GTCGTAACA (SEQ ID NO: 295) |
| HR5X-ACTB-834 | GGCGACGTAGCACAGCTTCTTATACGTCGAGTTGAA |
| | CGTCGTAACA (SEQ ID NO: 296) |
| HR5X-ACTB-603 | CGTACAGGGATAGCACAGCCTTATACGTCGAGTTGA |
| | ACGTCGTAACA (SEQ ID NO: 297) |
| HR5X-ACTB-363 | CCTCTCTTGCTCTGGGCCTTATACGTCGAGTTGAACG |
| | TCGTAACA (SEQ ID NO: 298) |
| HL5X-ACTB-1533 | TAGCGCTAACAACTTACGTCGTTATGTCGGCCACAT |
| | TGTGAACTT (SEQ ID NO: 299) |
| HR5X-ACTB-1533 | CAACAATGTGCAATC AAAGTCCTTATACGTCGAGTT |
| | GAACGTCGTAACA (SEQ ID NO: 300) |
| HL5X-ACTB-1819 | TAGCGCTAACAACTTACGTCGTTATGACATCTCAAG |
| | TTGGGGGACA (SEQ ID NO: 301) |
| HR5X-ACTB-1839 | GGGAGACCAAAAGCCTTCATTTATACGTCGAGTTGA |
| | ACGTCGTAACA (SEQ ID NO: 302) |
| HL2X-ACTB-1678 | TCGTACGTCTAACTTACGTCGTTATGTCCCCTGTGTG |
| | GACTTGG (SEQ ID NO: 303) |
| HR2X-ACTB-1678 | CACGAAAGCAATGCTATCACCTTATACGTCGAGTTG |
| | AAGAACAACCTG (SEQ ID NO: 304) |
| HL2X-ACTB-463 | TCGTACGTCTAACTTACGTCGTTATGAGAAGGTGTG |
| | GTGCCAGATT (SEQ ID NO: 305) |
| HR2X-ACTB-481 | CCACACGCAGCTCATTGTTTATACGTCGAGTTGAAG |
| | AACAACCTG (SEQ ID NO: 306) |
| HL1X-ACTB-463 | GACGCTAATAGACTTACGTCGTTATGAGAAGGTGTG |
| | GTGCCAGATT (SEQ ID NO: 307) |
| HR1X-ACTB-481 | CCACACGCAGCTCATTGTTTATACGTCGAGTTGATA |
| | GACACTCTT (SEQ ID NO: 308) |
| HL2X-UBC-2636 | TCGTACGTCTAACTTACGTCGTTATGTTAAAAGGGG |
| | AAACTTAGACACCC (SEQ ID NO: 309) |
| HR2X-UBC-2659 | GTGCAATGAAATTTGTTGAAACCTTATACGTCGAGT |
| | TGAAGAACAACCTG (SEQ ID NO: 310) |
| HL2X-UBC-2482 | TCGTACGTCTAACTTACGTCGTTATGTTGGATCTTTG |
| | CCTTGACATT (SEQ ID NO: 311) |
| HR2X-UBC-2482 | GGAGGGATGCCTTCCTTATCTTATACGTCGAGTTGA |
| | AGAACAACCTG (SEQ ID NO: 312) |
| HL6X-UBC-2055 | TAGGTCAGGAAACTTACGTCGTTATGTGCTGGTCAG |
| | GAGGGATG (SEQ ID NO: 313) |
| HR6X-UBC-2055 | TCCCAGCAAAGATCAACCTCTTATACGTCGAGTTGA |
| | ATAGCCAGGTT (SEQ ID NO: 314) |
| HL5X-UBC-2637 | TAGCGCTAACAACTTACGTCGTTATGTTAAAAGGGG |
| | AAACTTAGACACCC (SEQ ID NO: 315) |
| HL5X-UBC-2580 | TAGCGCTAACAACTTACGTCGTTATGTCTTTCTGGAT |
| | GTTGTAGTCAGACA (SEQ ID NO: 316) |
| HL5X-UBC-2535 | TAGCGCTAACAACTTACGTCGTTATGTGTTTCCCAG |
| | CAAAGATCAA (SEQ ID NO: 317) |
| HL5X-UBC-2491 | TAGCGCTAACAACTTACGTCGTTATGTTCCTTATCTT |
| | GGATCTTTGCC (SEQ ID NO: 318) |
| HL5X-UBC-2381 | TAGCGCTAACAACTTACGTCGTTATGTGAGACGGAG |
| | CACCAGGT (SEQ ID NO: 319) |
| HL5X-UBC-2314 | TAGCGCTAACAACTTACGTCGTTATGTTCCAGCTGTT |
| | TCCCAGC (SEQ ID NO: 320) |
| HL5X-UBC-2283 | TAGCGCTAACAACTTACGTCGTTATGTGCTGGTCAG |
| | GAGGGATG (SEQ ID NO: 321) |
| HL5X-UBC-2232 | TAGCGCTAACAACTTACGTCGTTATGTCAATGGTGT |
| | CACTCGGCT (SEQ ID NO: 322) |
| HL5X-UBC-2153 | TAGCGCTAACAACTTACGTCGTTATGTAAGACGGAG |
| | CACCAGGTG (SEQ ID NO: 323) |
| HR5X-UBC-2637 | TGCAATGAAATTTGTTGAAACCTTATACGTCGAGTT |
| | GAACGTCGTAACA (SEQ ID NO: 324) |
| HR5X-UBC-2580 | GGACCAAGTGCAGAGTGGACTTATACGTCGAGTTGA |
| | ACGTCGTAACA (SEQ ID NO: 325) |
| HR5X-UBC-2535 | GTGCGTCCATCTTCCAGCTTATACGTCGAGTTGAAC |
| | GTCGTAACA (SEQ ID NO: 326) |
| HR5X-UBC-2491 | CTGATCAGGAGGGATGCCTTATACGTCGAGTTGAAC |
| | GTCGTAACA (SEQ ID NO: 327) |
| HR5X-UBC-2381 | GAAGATTTGCATCCCACCTCTTATACGTCGAGTTGA |
| | ACGTCGTAACA (SEQ ID NO: 328) |
| HR5X-UBC-2314 | AGACAGGGTGCGTCCATCTTATACGTCGAGTTGAAC |
| | GTCGTAACA (SEQ ID NO: 329) |
| HR5X-UBC-2283 | TCCCAGCAAAGATCAACCTCTTATACGTCGAGTTGA |
| | ACGTCGTAACA (SEQ ID NO: 330) |
| HR5X-UBC-2232 | TGGATCTTTGCCTTGACATTCTTATACGTCGAGTTGA |
| | ACGTCGTAACA (SEQ ID NO: 331) |
| HR5X-UBC-2153 | GAAGATCTGCATCCCACCTCTTATACGTCGAGTTGA |
| | ACGTCGTAACA (SEQ ID NO: 332) |
| HL5X-UBC-2435 | TAGCGCTAACAACTTACGTCGTTATGTCGAGGGTGA |
| | TGGTCTTACC (SEQ ID NO: 333) |
| HR5X-UBC-2453 | GTGTCACTGGGCTCCACCTTATACGTCGAGTTGAAC |
| | GTCGTAACA (SEQ ID NO: 334) |
| HL1X-UBC-2636 | GACGCTAATAGACTTACGTCGTTATGTTAAAAGGGG |
| | AAACTTAGACACCC (SEQ ID NO: 335) |
| HR1X-UBC-2659 | GTGCAATGAAATTTGTTGAAACCTTATACGTCGAGT |
| | TGATAGACACTCTT (SEQ ID NO: 336) |
| HL3X-PPIA-1397 | TATTCGTTCGAACTTACGTCGTTATGTCAGGAGGCT |
| | GAGGCACTAG (SEQ ID NO: 337) |
| HR3X-PPIA-1397 | ACGCCCATTATCCCAGCTACTTATACGTCGAGTTGA |
| | CCGACGTATTG (SEQ ID NO: 338) |
| HL5X-PPIA-2201 | TAGCGCTAACAACTTACGTCGTTATGTCAGGTCTGA |
| | GCCACAAGTACA (SEQ ID NO: 339) |
| HL5X-PPIA-2124 | TAGCGCTAACAACTTACGTCGTTATGTGAGATGCAC |
| | AAGTGGTGGT (SEQ ID NO: 340) |
| HL5X-PPIA-2028 | TAGCGCTAACAACTTACGTCGTTATGTCTAGGATAG |
| | GCAAACTTGATTGC (SEQ ID NO: 341) |
| HL5X-PPIA-1982 | TAGCGCTAACAACTTACGTCGTTATGTGTTAAGGTG |
| | GGCAGAGAAG (SEQ ID NO: 342) |
| HL5X-PPIA-1883 | TAGCGCTAACAACTTACGTCGTTATGTTAGTGGGAA |
| | GGGTATCCCTTT (SEQ ID NO: 343) |
| HL5X-PPIA-1820 | TAGCGCTAACAACTTACGTCGTTATGTCAAAGCCTC |
| | CATAACCAAA (SEQ ID NO: 344) |
| HL5X-PPIA-1732 | TAGCGCTAACAACTTACGTCGTTATGTGCAGGGAGA |
| | CTGACTGTAGC (SEQ ID NO: 345) |
| HR5X-PPIA-2201 | GATGTTTATTTCCACCTTGCACTTATACGTCGAGTTG |
| | AACGTCGTAACA (SEQ ID NO: 346) |
| HR5X-PPIA-2124 | CGTAACCAGACAACACACAAGACTTATACGTCGAGT |
| | TGAACGTCGTAACA (SEQ ID NO: 347) |
| HR5X-PPIA-2028 | AATGATCAAATCCGCCACCTTATACGTCGAGTTGAA |
| | CGTCGTAACA (SEQ ID NO: 348) |
| HR5X-PPIA-1982 | TGGGTTAAGAACCCTAGAGGTCTTATACGTCGAGTT |
| | GAACGTCGTAACA (SEQ ID NO: 349) |
| HR5X-PPIA-1883 | AATTTCCTGAGAAACCAAGTCCTTATACGTCGAGTT |
| | GAACGTCGTAACA (SEQ ID NO: 350) |
| HR5X-PPIA-1820 | TTGGTCAGGTTTGCAAAACCTTATACGTCGAGTTGA |
| | ACGTCGTAACA (SEQ ID NO: 351) |
| HR5X-PPIA-1732 | TAGTCTGCGCCTTAACCCTCTTATACGTCGAGTTGA |
| | ACGTCGTAACA (SEQ ID NO: 352) |
| HL5X-PPIA-378 | TAGCGCTAACAACTTACGTCGTTATGTGGGAACCAT |
| | TTGTGTTGG (SEQ ID NO: 353) |
| HR5X-PPIA-378 | TGGCAGTGCAGATGAAAAACTTATACGTCGAGTTGA |
| | ACGTCGTAACA (SEQ ID NO: 354) |
| HL5X-PPIA-655 | TAGCGCTAACAACTTACGTCGTTATGTGCAGCGAGA |
| | GCACAAAG (SEQ ID NO: 355) |
| HR5X-PPIA-655 | ACATGGAACCCAAAGGGAACTTATACGTCGAGTTG |
| | AACGTCGTAACA (SEQ ID NO: 356) |
| HL5X-PPIA-588 | TAGCGCTAACAACTTACGTCGTTATGTACAGAAGGA |
| | ATGATCTGGTGG (SEQ ID NO: 357) |
| HR5X-PPIA-606 | AGGGGTGCTCTCCTGAGCTTATACGTCGAGTTGAAC |
| | GTCGTAACA (SEQ ID NO: 358) |
| HL2X-PPIA-473 | TCGTACGTCTAACTTACGTCGTTATGTCCATGGCCTC |
| | CACAATATT (SEQ ID NO: 359) |
| HR2X-PPIA-489 | CCTGGACCCAAAGCGCTTATACGTCGAGTTGAAGAA |
| | CAACCTG (SEQ ID NO: 360) |
| HL1X-PPIA-473 | GACGCTAATAGACTTACGTCGTTATGTCCATGGCCT |
| | CCACAATATT (SEQ ID NO: 361) |
| HR1X-PPIA-489 | CCTGGACCCAAAGCGCTTATACGTCGAGTTGATAGA |
| | CACTCTT (SEQ ID NO: 362) |
| HL5X-SOX4-5611 | TAGCGCTAACAACTTACGTCGTTATGTTAAAAAGGC |
| | AGACGTCCTAGTG (SEQ ID NO: 363) |
| HR5X-SOX4-5611 | AAACCCTTAACGGAACTGCCTTATACGTCGAGTTGA |
| | ACGTCGTAACA (SEQ ID NO: 364) |
| HL5X-SOX4-492 | TAGCGCTAACAACTTACGTCGTTATGTCTGGTGCAA |
| | AATCTGTTCC (SEQ ID NO: 365) |
| HR5X-SOX4-492 | GCATTGGAATAAAGAATCAGCCTTATACGTCGAGTT |
| | GAACGTCGTAACA (SEQ ID NO: 366) |
| HL5X-SOX4-4150 | TAGCGCTAACAACTTACGTCGTTATGTGCCCCTGTA |
| | CCACATCAA (SEQ ID NO: 367) |
| HR5X-SOX4-4169 | CACTCGCCCTCCACTGACTTATACGTCGAGTTGAAC |
| | GTCGTAACA (SEQ ID NO: 368) |
| HL5X-SOX4-1521 | TAGCGCTAACAACTTACGTCGTTATGTCTCTCCCTCT |
| | CTCTCGCTCT (SEQ ID NO: 369) |
| HR5X-SOX4-1521 | TCCCAGGCTGGAGAGTCTCTTATACGTCGAGTTGAA |
| | CGTCGTAACA (SEQ ID NO: 370) |
| HL5X-SOX4-2074 | TAGCGCTAACAACTTACGTCGTTATGTGTCTTTGAG |
| | CAGCTTCCAG (SEQ ID NO: 371) |
| HR5X-SOX4-2074 | AATGAAAGGGATCTTGTCGCTTATACGTCGAGTTGA |
| | ACGTCGTAACA (SEQ ID NO: 372) |
| HL5X-SOX4-3011 | TAGCGCTAACAACTTACGTCGTTATGTCAAAGTTTG |
| | AGCTGGGGTT (SEQ ID NO: 373) |
| HR5X-SOX4-3011 | CTGCCCAGGGACATGCTCTTATACGTCGAGTTGAAC |
| | GTCGTAACA (SEQ ID NO: 374) |
| HL5X-S0X4-3450 | TAGCGCTAACAACTTACGTCGTTATGTCTTCCTCCTC |
| | CGCGTC (SEQ ID NO: 375) |
| HR5X-SOX4-3450 | GTCGCCCCTGTCTACCCTTATACGTCGAGTTGAACG |
| | TCGTAACA (SEQ ID NO: 376) |
| HL5X-SOX4-3809 | TAGCGCTAACAACTTACGTCGTTATGTCAGTCCCGG |
| | CGTTCC (SEQ ID NO: 377) |
| HR5X-SOX4-3809 | CGCCTGCGTGGAGTCTCTTATACGTCGAGTTGAACG |
| | TCGTAACA (SEQ ID NO: 378) |
| HL5X-SOX4-4009 | TAGCGCTAACAACTTACGTCGTTATGTTCCAGTTCGT |
| | GTCCTCCTC (SEQ ID NO: 379) |
| HR5X-SOX4-4009 | AGTTTGACCGTGAACCCCCTTATACGTCGAGTTGAA |
| | CGTCGTAACA (SEQ ID NO: 380) |
| HL5X-SOX4-5145 | TAGCGCTAACAACTTACGTCGTTATGTTTTTACAAG |
| | ACGGGGGCC (SEQ ID NO: 381) |
| HR5X-SOX4-5145 | TTTGGCTAATTCTCCTCCCCTTATACGTCGAGTTGAA |
| | CGTCGTAACA (SEQ ID NO: 382) |
| HL2X-SOX4-4169 | TCGTACGTCTAACTTACGTCGTTATGTGCCCCTGTAC |
| | CACATCAA (SEQ ID NO: 383) |
| HR2X-SOX4-4169 | CACTCGCCCTCCACTGACTTATACGTCGAGTTGAAG |
| | AACAACCTG (SEQ ID NO: 384) |
| HL2X-SOX4-5611 | TCGTACGTCTAACTTACGTCGTTATGTTAAAAAGGC |
| | AGACGTCCTAGTG (SEQ ID NO: 385) |
| HR2X-SOX4-5611 | AAACCCTTAACGGAACTGCCTTATACGTCGAGTTGA |
| | AGAACAACCTG (SEQ ID NO: 386) |
| HL2X-SOX4-3908 | TCGTACGTCTAACTTACGTCGTTATGTCCTCCCCTCA |
| | ACATCTCCT (SEQ ID NO: 387) |
| HR2X-SOX4-3908 | GGTCACACTGGCTGGCCTTATACGTCGAGTTGAAGA |
| | ACAACCTG (SEQ ID NO: 388) |
| HL2X-SOX4-1449 | TCGTACGTCTAACTTACGTCGTTATGTGAGTCTGGA |
| | GACCGTGCTA (SEQ ID NO: 389) |
| HR2X-SOX4-1469 | CAGTTTGCTGTCTCTCGGCTTATACGTCGAGTTGAA |
| | GAACAACCTG (SEQ ID NO: 390) |
| HL2X-SOX4-1933 | TCGTACGTCTAACTTACGTCGTTATGTCGGGGTCTTG |
| | CACCA (SEQ ID NO: 391) |
| HR2X-SOX4-1933 | GTCGCTTGATGTGCCCACTTATACGTCGAGTTGAAG |
| | AACAACCTG (SEQ ID NO: 392) |
| HL2X-ID2-107 | TCGTACGTCTAACTTACGTCGTTATGTAGCTGCGCTT |
| | GGCACC (SEQ ID NO: 393) |
| HR2X-ID2-107 | TGCCGCCTGCTGAGCTTATACGTCGAGTTGAAGAAC |
| | AACCTG (SEQ ID NO: 394) |
| HL2X-ID2-224 | TCGTACGTCTAACTTACGTCGTTATGTGTTTTTCCTA |
| | ACGGACCTCA (SEQ ID NO: 395) |
| HR2X-ID2-224 | GCTGTGGTCCGACAGGCTTATACGTCGAGTTGAAGA |
| | ACAACCTG (SEQ ID NO: 396) |
| HL2X-ID2-284 | TCGTACGTCTAACTTACGTCGTTATGTCATCGGGTCG |
| | TCCACA (SEQ ID NO: 397) |
| HR2X-ID2-284 | TCGTTCATGTTGTATAGCAGGCTTATACGTCGAGTT |
| | GAAGAACAACCTG (SEQ ID NO: 398) |
| HL2X-ID2-411 | TCGTACGTCTAACTTACGTCGTTATGTGCAGGTCCA |
| | AGATGTAGTCG (SEQ ID NO: 399) |
| HR2X-ID2-411 | CGAGTCCAGGGCGATCTTATACGTCGAGTTGAAGAA |
| | CAACCTG (SEQ ID NO: 400) |
| HL2X-ID2-854 | TCGTACGTCTAACTTACGTCGTTATGTCCCCATGGTG |
| | GGAATAGT (SEQ ID NO: 401) |
| HR2X-ID2-873 | CTTGTGATTTTAACGTTTTCGCTTATACGTCGAGTTG |
| | AAGAACAACCTG (SEQ ID NO: 402) |
| HL5X-ID2-149 | TAGCGCTAACAACTTACGTCGTTATGTGGCTGCCCT |
| | GAAGCTC (SEQ ID NO: 403) |
| HR5X-ID2-149 | GAGACCGGGAGGGAGCTTATACGTCGAGTTGAACG |
| | TCGTAACA (SEQ ID NO: 404) |
| HR5X-ID2-190 | ACCTCACGGGACTGAAGGCTTATACGTCGAGTTGAA |
| | CGTCGTAACA (SEQ ID NO: 405) |
| HL5X-ID2-197 | TAGCGCTAACAACTTACGTCGTTATGTGAAGGCTTT |
| | CATGCTGACC (SEQ ID NO: 406) |
| HL5X-ID2-362 | TAGCGCTAACAACTTACGTCGTTATGTCACCTTCTTG |
| | TTCTGGGGG (SEQ ID NO: 407) |
| HR5X-ID2-362 | CTGCAGGATTTCCATCTTGCTTATACGTCGAGTTGA |
| | ACGTCGTAACA (SEQ ID NO: 408) |
| HL5X-ID2-468 | TAGCGCTAACAACTTACGTCGTTATGTGCCCGGGTC |
| | TCTGGT (SEQ ID NO: 409) |
| HR5X-ID2-468 | GTCCTGGACGCCTGGTTCTTATACGTCGAGTTGAAC |
| | GTCGTAACA (SEQ ID NO: 410) |
| HL5X-ID2-552 | TAGCGCTAACAACTTACGTCGTTATGTCAGAAGGGA |
| | ATTCAGAAGCC (SEQ ID NO: 411) |
| HR5X-ID2-552 | GCTTTGCTGTCATTTGACATTAACTTATACGTCGAGT |
| | TGAACGTCGTAACA (SEQ ID NO: 412) |
| HL5X-ID2-593 | TAGCGCTAACAACTTACGTCGTTATGTTATTCAGCC |
| | ACACAGTGCTTT (SEQ ID NO: 413) |
| HR5X-ID2-593 | AAAAGAAATCATGAACACCGCTTATACGTCGAGTTG |
| | AACGTCGTAACA (SEQ ID NO: 414) |
| HL5X-ID2-694 | TAGCGCTAACAACTTACGTCGTTATGTCCTTGTGAA |
| | ATGGTTGAAAAA (SEQ ID NO: 415) |
| HR5X-ID2-694 | AAAAGGTCCATTCAACTTGTCCTTATACGTCGAGTT |
| | GAACGTCGTAACA (SEQ ID NO: 416) |
| HL5X-ID2-951 | TAGCGCTAACAACTTACGTCGTTATGTGGTATTCAC |
| | GCTCCACCTT (SEQ ID NO: 417) |
| HR5X-ID2-951 | AAGTGACTGAATACTGGATCCTTCTTATACGTCGAG |
| | TTGAACGTCGTAACA (SEQ ID NO: 418) |
| HL5X-ID2-1233 | TAGCGCTAACAACTTACGTCGTTATGTCTGAAATAA |
| | AGCAGGCAATCA (SEQ ID NO: 419) |
| HR5X-ID2-1255 | AATAAAAATCAAAGCACTGGTCCTTATACGTCGAGT |
| | TGAACGTCGTAACA (SEQ ID NO: 420) |
| HL5X-ID2-411 | TAGCGCTAACAACTTACGTCGTTATGTGCAGGTCCA |
| | AGATGTAGTCG (SEQ ID NO: 421) |
| HR5X-ID2-411 | CGAGTCCAGGGCGATCTTATACGTCGAGTTGAACGT |
| | CGTAACA (SEQ ID NO: 422) |
| HL2X-CTNNB1-1615 | TCGTACGTCTAACTTACGTCGTTATGTCTATACCACC |
| | CACTTGGCAG (SEQ ID NO: 423) |
| HR2X-CTNNB1-1615 | GGACAGTACGC ACAAGAGCCTTATACGTCGAGTTGA |
| | AGAACAACCTG (SEQ ID NO: 424) |
| HL2X-CTNNB1-2599 | TCGTACGTCTAACTTACGTCGTTATGTGATTGCTGTC |
| | ACCTGGAGG (SEQ ID NO: 425) |
| HR2X-CTNNB1-2599 | CAGTATCAAACC AGGCCAGCTTATACGTCGAGTTGA |
| | AGAACAACCTG (SEQ ID NO: 426) |
| HL2X-CTNNB1-2732 | TCGTACGTCTAACTTACGTCGTTATGTAAACCACTCC |
| | CACCCTACC (SEQ ID NO: 427) |
| HR2X-CTNNB1-2732 | TGTGGCAGATTTAC AAATAGCCTTATACGTCGAGTT |
| | GAAGAACAACCTG (SEQ ID NO: 428) |
| HL2X-CTNNB1-3000 | TCGTACGTCTAACTTACGTCGTTATGTCGAGCCCTCT |
| | CAGCAAC (SEQ ID NO: 429) |
| HR2X-CTNNB1-3000 | GAGATACCAGCCCACCCCTTATACGTCGAGTTGAAG |
| | AACAACCTG (SEQ ID NO: 430) |
| HL2X-CTNNB1-3021 | TCGTACGTCTAACTTACGTCGTTATGTTGGTTAGTGT |
| | GTCAGGCACTT (SEQ ID NO: 431) |
| HR2X-CTNNB1-3041 | TGTTCCCATAGGAAACTCAGCTTATACGTCGAGTTG |
| | AAGAACAACCTG (SEQ ID NO: 432) |
| HL5X-CTNNB1-149 | TAGCGCTAACAACTTACGTCGTTATGTCAGACCTTC |
| | CTCCGTCTCC (SEQ ID NO: 433) |
| HR5X-CTNNB1-149 | GGGGACTGAAGCTGCTCCTTATACGTCGAGTTGAAC |
| | GTCGTAACA (SEQ ID NO: 434) |
| HL5X-CTNNB1-348 | TAGCGCTAACAACTTACGTCGTTATGTAACAGCCGC |
| | TTTTCTGTCTG (SEQ ID NO: 435) |
| HR5X-CTNNB1-348 | AGACTGTTGCTGCC AGTGACTTATACGTCGAGTTGA |
| | ACGTCGTAACA (SEQ ID NO: 436) |
| HL5X-CTNNB1-958 | TAGCGCTAACAACTTACGTCGTTATGTCACGATGAT |
| | GGGAAAGGTT (SEQ ID NO: 437) |
| HR5X-CTNNB1-958 | TAAAGATGGCCAGTAAGCCCTTATACGTCGAGTTGA |
| | ACGTCGTAACA (SEQ ID NO: 438) |
| HL5X-CTNNB1-2117 | TAGCGCTAACAACTTACGTCGTTATGTCTTTGGATGT |
| | TTTCAATGGG (SEQ ID NO: 439) |
| HR5X-CTNNB1-2117 | AGAGGACCCCTGCAGCTACTTATACGTCGAGTTGAA |
| | CGTCGTAACA (SEQ ID NO: 440) |
| HL5X-CTNNB1-2528 | TAGCGCTAACAACTTACGTCGTTATGTGGATAGTCA |
| | GCACCAGGGT (SEQ ID NO: 441) |
| HR5X-CTNNB1-2528 | ATCTGGC AGCCCATCAACTTATACGTCGAGTTGAAC |
| | GTCGTAACA (SEQ ID NO: 442) |
| HL5X-CTNNB1-3105 | TAGCGCTAACAACTTACGTCGTTATGTCCTCGACCA |
| | AAAAGGACC (SEQ ID NO: 443) |
| HR5X-CTNNB1-3105 | TCCCAAAATCCATTTGTATTGTTACTTATACGTCGAG |
| | TTGAACGTCGTAACA (SEQ ID NO: 444) |
| HL5X-CTNNB1-3147 | TAGCGCTAACAACTTACGTCGTTATGTTCACTTCTTG |
| | AGTCACTCCCA (SEQ ID NO: 445) |
| HR5X-CTNNB1-3147 | TGTGATCCATTCTTGTGCATTCTTATACGTCGAGTTG |
| | AACGTCGTAACA (SEQ ID NO: 446) |
| HL5X-CTNNB1-3618 | TAGCGCTAACAACTTACGTCGTTATGTGTCCAAAAC |
| | AAGGTTCCAA (SEQ ID NO: 447) |
| HR5X-CTNNB1-3618 | GGGATAAAAGGCAACTGGTAAACTTATACGTCGAG |
| | TTGAACGTCGTAACA (SEQ ID NO: 448) |
| HL5X-CTNNB1-2580 | TAGCGCTAACAACTTACGTCGTTATGTGATTGCTGT |
| | CACCTGGAG (SEQ ID NO: 449) |
| HR5X-CTNNB1-2599 | CAGTATCAAACC AGGCCAGCTTATACGTCGAGTTGA |
| | ACGTCGTAACA (SEQ ID NO: 450) |
| HL5X-CTNNB1-3000 | TAGCGCTAACAACTTACGTCGTTATGTCGAGCCCTC |
| | TCAGCAAC (SEQ ID NO: 451) |
| HR5X-CTNNB1-3000 | GAGATACCAGCCCACCCCTTATACGTCGAGTTGAAC |
| | GTCGTAACA (SEQ ID NO: 452) |
| HL2X-Neg20 | TCGTACGTCTAACTTACGTCGTTATGAACCGGTACG |
| | TACGGATTGA (SEQ ID NO: 453) |
| HR2X-Neg20 | AACCGGTACGTACGGATTGATTATACGTCGAGTTGA |
| | AGAACAACCTG (SEQ ID NO: 454) |
| HL5X-Neg20 | TAGCGCTAACAACTTACGTCGTTATGAACCGGTACG |
| | TACGGATTGA (SEQ ID NO: 455) |
| HR5X-Neg20 | AACCGGTACGTACGGATTGATTATACGTCGAGTTGA |
| | ACGTCGTAACA (SEQ ID NO: 456) |
| HL4X-SCGB1A1a- 91 | TTAGTAGGCGAACTTACGTCGTTATGTGCAGCAGAG |
| | AGCCAGTGTG (SEQ ID NO: 457) |
| HR4X-SCGB1A1a- 112 | TTAGTAGGCGAACTTACGTCGTTATGAACTGGAGGG |
| | TGTGTCCATG (SEQ ID NO: 458) |
| HL4X-SCGB1A1b-166 | AAGTTCCATGGCAGCCTCATTTATACGTCGAGTTGA |
| | ACATAAGTGCG (SEQ ID NO: 459) |
| HR4X-SCGV1A1b-187 | TTAGTAGGCGAACTTACGTCGTTATGTTAATGATGC |
| | TTTCTCTGGG (SEQ ID NO: 460) |
| HL4X-SCGB1A1c-278 | GGGCTATTTTTTCCATGAGCTTATACGTCGAGTTGA |
| | ACATAAGTGCG (SEQ ID NO: 461) |
| HR4X-SCGB1A1c-299 | GGGCTATTTTTTCCATGAGCTTATACGTCGAGTTGA |
| | ACATAAGTGCG (SEQ ID NO: 462) |
| HL4X-SCGB1A1d-374 | TTAGTAGGCGAACTTACGTCGTTATGTCAAAGCATG |
| | GCAGCGGCAG (SEQ ID NO: 463) |
| HR4X-SCGB1A1d-395 | CAAGGCTGGTGGGCGTGGACTTATACGTCGAGTTGA |
| | ACATAAGTGC (SEQ ID NO: 464) |

While the preferred embodiments of the invention have been illustrated and described, it will be appreciated that various changes can be made therein without departing from the spirit and scope of the invention.

1. A method of detecting one or more target nucleic acids in a sample, the method comprising:
a) providing at least one probe set for each target nucleic acid, wherein each probe set comprises: i) a first probe comprising a 5′ overhang region and a region that hybridizes to the target nucleic acid at a first target site; ii) a second probe comprising a 3′ overhang region and a region that hybridizes to the target nucleic acid at a second target site;
b) contacting the sample with the probe sets;
c) adding at least one bridge oligonucleotide to the sample for each probe set, wherein the bridge oligonucleotide comprises i) a first portion that hybridizes to a complementary portion in the 5′ overhang region of the first probe of the probe set, and ii) a second portion that hybridizes to a complementary portion in the 3′ overhang region of the second probe of the probe set, wherein the first probe and the second probe, when bound to one of the target nucleic acids, are in sufficient proximity to each other to simultaneously hybridize to the bridge oligonucleotide;
d) adding at least one circle oligonucleotide to the sample for each probe set, wherein the circle oligonucleotide comprises a first portion that hybridizes to a complementary region at the 5′ end of the 5′ overhang region of the first probe of the probe set, and a second portion that hybridizes to a complementary region at the 3′ end of the 3′ overhang region of the second probe of the probe set;
e) forming circular DNA where any two probes of a probe set bind sufficiently close to each other on one of the target nucleic acids to allow ligation of the bridge oligonucleotide and circle oligonucleotide that are hybridized to the two probes to generate a closed circle;
f) performing rolling circle amplification, wherein each circular DNA molecule formed serves as a template to produce a concatemer comprising multiple copies of the circular DNA nucleotide sequence;
g) contacting each concatemer with one or more imager oligonucleotides, wherein each imager oligonucleotide comprises a detectable label and a nucleotide sequence complementary to one or more sites in the circular DNA sequence, wherein the imager oligonucleotide binds to said sites in the multiple copies of the circular DNA sequence of the concatemer; and
h) detecting the bound imager oligonucleotides.

2. (canceled)

3. The method of claim 1, wherein the second target site is adjacent to the first target site on the target nucleic acid.

4. The method of claim 3, wherein the first target site and the second target site are contiguous on the target nucleic acid.

5. The method of claim 1, wherein a plurality of probe sets comprising probes capable of hybridizing at a plurality of target sites on a single target nucleic acid are used.

6. The method of claim 1, wherein each probe has a similar melting temperature (Tₘ) for binding to its cognate target site.

7. The method of claim 6, wherein the Tₘ ranges from about 45° C. to about 65° C.

8. The method of claim 1, wherein the target nucleic acid is RNA or DNA.

9-12. (canceled)

13. The method of claim 1, wherein the detectable label is a fluorophore.

14. The method of claim 1, wherein the one or more target nucleic acids are in a cell, a population of cells, a tissue, or an organ.

15. (canceled)

16. The method of claim 14, further comprising mapping an anatomical location for at least one target nucleic acid in the tissue or organ.

17. The method of claim 14, wherein the cell is a eukaryotic cell, a prokaryotic cell, an archaeon cell, or an artificial cell.

18-19. (canceled)

20. The method of claim 14, wherein the cell is a fixed cell or a live cell.

21. (canceled)

22. The method of claim 14, wherein the cell is exposed to a test condition prior to said contacting the sample with one or more probe sets.

23. The method of claim 22, wherein the test condition comprises exposing the cell to a drug, a ligand for a receptor, a hormone, a second messenger, a pathogen, a genetic modification, a change in temperature, a change in growth media, a change in membrane potential, or a change in osmotic pressure.

24. (canceled)

25. The method of claim 1, wherein a plurality of probe sets comprising probes capable of hybridizing at a plurality of target sites on multiple target nucleic acids are used for multiplexed detection of a plurality of target nucleic acids.

26. The method of claim 25, further comprising using a plurality of circle oligonucleotides, wherein each circle oligonucleotide binds to a different probe set.

27. The method of claim 26, further comprising using a plurality of imager oligonucleotides, wherein each imager oligonucleotide comprises a different detectable label.

28. The method of claim 27, wherein each circle oligonucleotide comprises one or more binding sites for a different imager oligonucleotide, such that different circle oligonucleotides are bound by different imager oligonucleotides comprising different detectable labels to allow different target nucleic acids to be detectably distinguished from one another.

29. (canceled)

30. The method of claim 28, wherein the detectable labels are fluorescent labels, bioluminescent labels, chemiluminescent labels, isotopic labels, nanoparticles, or metals.

31. The method of claim 30, wherein the fluorescent labels are detected by performing fluorescence imaging.

32. The method of claim 31, wherein said detecting is performed using multiple cycles of fluorescence imaging to allow detection of subsets of the target nucleic acids sequentially.

33. The method of claim 32, wherein the subsets of the target nucleic acids are detected sequentially by a method comprising:
a) contacting the sample with a subset of the imager oligonucleotides;
b) performing a cycle of fluorescence imaging;
c) removing the imager oligonucleotides from the sample;
d) contacting the sample with another subset of the imager oligonucleotides;
e) performing another cycle of fluorescence imaging; and
f) removing the imager oligonucleotides from the sample.

34-38. (canceled)

39. The method of claim 28, further comprising identifying at least one cell type based on detection of one or more target nucleic acids.

40. The method of claim 39, wherein said identifying is automated by using an algorithm for cell classification.

41. The method of claim 40, wherein the algorithm is a clustering algorithm or a machine learning algorithm.

42. (canceled)

43. A composition for detecting one or more target nucleic acids in a sample comprising:
a) at least one probe set for each target nucleic acid, wherein each probe set comprises: i) a first probe comprising a 5′ overhang region and a region that hybridizes to a target nucleic acid at a first target site; ii) a second probe comprising a 3′ overhang region and a region that hybridizes to the target nucleic acid at a second target site;
b) at least one bridge oligonucleotide for each probe set, wherein the bridge oligonucleotide comprises i) a first portion capable of hybridizing to a complementary portion in the 5′ overhang region of the first probe of the probe set, and ii) a second portion capable of hybridizing to a complementary portion in the 3′ overhang region of the second probe of the probe set, wherein the first probe and the second probe, when bound to one of the target nucleic acids, are in sufficient proximity to each other to simultaneously hybridize to the bridge oligonucleotide; and
c) at least one circle oligonucleotide for each probe set, wherein the circle oligonucleotide comprises a first portion capable of hybridizing to a complementary region at the 5′ end of the 5′ overhang region of the first probe of the probe set, and a second portion capable of hybridizing to a complementary region at the 3′ end of the 3′ overhang region of the second probe of the probe set.

44. A kit comprising the composition of claim 43 and instructions for detecting the one or more target nucleic acids.

45-46. (canceled)

47. An oligonucleotide selected from the group consisting of:
a) an oligonucleotide comprising a nucleotide sequence selected from the group consisting of SEQ ID NOS:1-464; and
b) an oligonucleotide comprising a nucleotide sequence having at least 95% identity to a sequence selected from the group consisting of SEQ ID NOS:1-464.


SEQUENCE LISTING (464 sequences)

SEQ ID NO: 1 - DNA, 60 bp, Artificial Sequence
  CC06-DP4 connector circle
  source (1)..(60): sequence is synthesized
ttcctgacctaacaaaacatgcgtctatttagtggagccacataattataacctggctat

SEQ ID NO: 2 - DNA, 60 bp, Artificial Sequence
  CC03-X1 connector circle
  source (1)..(60): sequence is synthesized
tcgaacgaataacaaagggagactgattacacgataacaacaatacacacaatacgtcgg

SEQ ID NO: 3 - DNA, 60 bp, Artificial Sequence
  CC02-Y1 connector circle
  source (1)..(60): sequence is synthesized
tagacgtacgagattaaaacatagaggcacttgagctagaatcatataacaggttgttct

SEQ ID NO: 4 - DNA, 60 bp, Artificial Sequence
  CC04-X4 connector circle
  source (1)..(60): sequence is synthesized
tcgcctactaaacaaagaacctccattaacatcgcaacaacaatacacacgcacttatgt

SEQ ID NO: 5 - DNA, 60 bp, Artificial Sequence
  CC05-DP5 connector circle
  source (1)..(60): sequence is synthesized
tgttagcgctaacaaaatgctgctgctgtactacgaacaacaatacacatgttacgacgt

SEQ ID NO: 6 - DNA, 60 bp, Artificial Sequence
  CC07-Y7 connector circle
  source (1)..(60): sequence is synthesized
gacaattacacgactaaaacatctcggtgcttgaactggtctcaactaacctgaggaata

SEQ ID NO: 7 - DNA, 62 bp, Artificial Sequence
  CC11X-X8 connector circle
  source (1)..(62): sequence is synthesized
taacacgaacactaaacaagtctaactaacgataccataaagaagaataaccgaatcaagat

SEQ ID NO: 8 - DNA, 62 bp, Artificial Sequence
  CC12X-X9mod connector circle
  source (1)..(62): sequence is synthesized
accatacttgaacaaaccacactcacatggatccactcgtaacaacaataacaggatacatc

SEQ ID NO: 9 - DNA, 31 bp, Artificial Sequence
  CB2-31AC common bridge
  source (1)..(31): sequence is synthesized
tcaactcgacgtataacataacgacgtaagt

SEQ ID NO: 10 - DNA, 20 bp, Artificial Sequence
  AF488-Y1
  source (1)..(20): sequence is synthesized
agaggcacttgagctagaat

SEQ ID NO: 11 - DNA, 21 bp, Artificial Sequence
  Cy5-DP5
  source (1)..(21): sequence is synthesized
aatgctgctgctgtactacgg

SEQ ID NO: 12 - DNA, 20 bp, Artificial Sequence
  X1-Cy3
  source (1)..(20): sequence is synthesized
gggagactgattacacgata

SEQ ID NO: 13 - DNA, 20 bp, Artificial Sequence
  TEX615-X4
  source (1)..(20): sequence is synthesized
agaacctccattaacatcgc

SEQ ID NO: 14 - RNA, 20 nt, Artificial Sequence
  uY7-A488
  source (1)..(20): sequence is synthesized
cucggugcuugaacuggucu

SEQ ID NO: 15 - RNA, 20 nt, Artificial Sequence
  uX8-Cy3
  source (1)..(20): sequence is synthesized
gucuaacuaacgauaccaua

SEQ ID NO: 16 - RNA, 19 nt, Artificial Sequence
  uX9-Texas red
  source (1)..(19): sequence is synthesized
cucacauggauccacucgu

SEQ ID NO: 17 - RNA, 21 nt, Artificial Sequence
  uDP4-Cy5
  source (1)..(21): sequence is synthesized
augcgucuauuuaguggagcc

SEQ ID NO: 18 - DNA, 26 bp, Artificial Sequence
  HL5X H probe
  source (1)..(26): sequence is synthesized
tagcgctaacaacttacgtcgttatg

SEQ ID NO: 19 - DNA, 27 bp, Artificial Sequence
  HR5X H probe
  source (1)..(27): sequence is synthesized
ttatacgtcgagttgaacgtcgtaaca

SEQ ID NO: 20 - DNA, 26 bp, Artificial Sequence
  HL2X H probe
  source (1)..(26): sequence is synthesized
tcgtacgtctaacttacgtcgttatg

SEQ ID NO: 21 - DNA, 27 bp, Artificial Sequence
  HR2X H probe
  source (1)..(27): sequence is synthesized
ttatacgtcgagttgaagaacaacctg

SEQ ID NO: 22 - DNA, 26 bp, Artificial Sequence
  HL3X H probe
  source (1)..(26): sequence is synthesized
tattcgttcgaacttacgtcgttatg

SEQ ID NO: 23 - DNA, 27 bp, Artificial Sequence
  HR3X H probe
  source (1)..(27): sequence is synthesized
ttatacgtcgagttgaccgacgtattg

SEQ ID NO: 24 - DNA, 26 bp, Artificial Sequence
  HL4X H probe
  source (1)..(26): sequence is synthesized
ttagtaggcgaacttacgtcgttatg

SEQ ID NO: 25 - DNA, 27 bp, Artificial Sequence
  HR4X H probe
  source (1)..(27): sequence is synthesized
ttatacgtcgagttgaacataagtgcg

SEQ ID NO: 26 - DNA, 26 bp, Artificial Sequence
  HL6X H probe
  source (1)..(26): sequence is synthesized
taggtcaggaaacttacgtcgttatg

SEQ ID NO: 27 - DNA, 27 bp, Artificial Sequence
  HR6X H probe
  source (1)..(27): sequence is synthesized
ttatacgtcgagttgaatagccaggtt

SEQ ID NO: 28 - DNA, 26 bp, Artificial Sequence
  HL7X H probe
  source (1)..(26): sequence is synthesized
gtgtaattgtcacttacgtcgttatg

SEQ ID NO: 29 - DNA, 27 bp, Artificial Sequence
  HR7X H probe
  source (1)..(27): sequence is synthesized
ttatacgtcgagttgatattcctcagg

SEQ ID NO: 30 - DNA, 26 bp, Artificial Sequence
  HL11X H probe
  source (1)..(26): sequence is synthesized
tgttcgtgttaacttacgtcgttatg

SEQ ID NO: 31 - DNA, 29 bp, Artificial Sequence
  HR11X H probe
  source (1)..(29): sequence is synthesized
ttatacgtcgagttgaatcttgattcggt

SEQ ID NO: 32 - DNA, 26 bp, Artificial Sequence
  HL12X H probe
  source (1)..(26): sequence is synthesized
tcaagtatggtacttacgtcgttatg

SEQ ID NO: 33 - DNA, 27 bp, Artificial Sequence
  HR12X H probe
  source (1)..(27): sequence is synthesized
ttatacgtcgagttgagatgtatcctg

SEQ ID NO: 34 - DNA, 46 bp, Artificial Sequence
  mm-HL4X-Ager-1335
  source (1)..(46): sequence is synthesized
ttagtaggcgaacttacgtcgttatgtctggttggagaaggaagtg

SEQ ID NO: 35 - DNA, 45 bp, Artificial Sequence
  mm-HR4X-Ager-1335
  source (1)..(45): sequence is synthesized
agcatggatcatgtgggcttatacgtcgagttgaacataagtgcg

SEQ ID NO: 36 - DNA, 45 bp, Artificial Sequence
  mm-HL4X-Ager-1280
  source (1)..(45): sequence is synthesized
ttagtaggcgaacttacgtcgttatgtgctctagggccatcacac

SEQ ID NO: 37 - DNA, 45 bp, Artificial Sequence
  mm-HR4X-Ager-1280
  source (1)..(45): sequence is synthesized
gatggaatgtgggggagcttatacgtcgagttgaacataagtgcg

SEQ ID NO: 38 - DNA, 45 bp, Artificial Sequence
  mm-HL4X-Ager-1031
  source (1)..(45): sequence is synthesized
ttagtaggcgaacttacgtcgttatgtcacccacagagccttcag

SEQ ID NO: 39 - DNA, 47 bp, Artificial Sequence
  mm-HR4X-Ager-1031
  source (1)..(47): sequence is synthesized
ctagcgtacccagcccagacttatacgtcgagttgaacataagtgcg

SEQ ID NO: 40 - DNA, 46 bp, Artificial Sequence
  mm-HL4X-Ager-771
  source (1)..(46): sequence is synthesized
ttagtaggcgaacttacgtcgttatgtattccaccttcaggctcaa

SEQ ID NO: 41 - DNA, 45 bp, Artificial Sequence
  mm-HR4X-Ager-771
  source (1)..(45): sequence is synthesized
agtcccaccaggagcgacttatacgtcgagttgaacataagtgcg

SEQ ID NO: 42 - DNA, 46 bp, Artificial Sequence
  mm-HL4X-Ager-652
  source (1)..(46): sequence is synthesized
ttagtaggcgaacttacgtcgttatgtgcaggagaaggtaggatgg

SEQ ID NO: 43 - DNA, 45 bp, Artificial Sequence
  mm-HR4X-Ager-652
  source (1)..(45): sequence is synthesized
gaaggcccaggctgaaacttatacgtcgagttgaacataagtgcg

SEQ ID NO: 44 - DNA, 46 bp, Artificial Sequence
  mm-HL4X-Ager-496
  source (1)..(46): sequence is synthesized
ttagtaggcgaacttacgtcgttatgtaagggtccctgcagggtag

SEQ ID NO: 45 - DNA, 47 bp, Artificial Sequence
  mm-HR4X-Ager-496
  source (1)..(47): sequence is synthesized
tttcccatctaagtgccagcttatacgtcgagttgaacataagtgcg

SEQ ID NO: 46 - DNA, 45 bp, Artificial Sequence
  mm-HL4X-Ager-436
  source (1)..(45): sequence is synthesized
ttagtaggcgaacttacgtcgttatgtggctgtgagttcagaggc

SEQ ID NO: 47 - DNA, 48 bp, Artificial Sequence
  mm-HR4X-Ager-436
  source (1)..(48): sequence is synthesized
tccccaccttattagggacacttatacgtcgagttgaacataagtgcg

SEQ ID NO: 48 - DNA, 45 bp, Artificial Sequence
  mm-HL4X-Ager-350
  source (1)..(45): sequence is synthesized
ttagtaggcgaacttacgtcgttatgttccctcgcctgttagttg

SEQ ID NO: 49 - DNA, 48 bp, Artificial Sequence
  mm-HR4X-Ager-350
  source (1)..(48): sequence is synthesized
cggtagttggacttgacctccttatacgtcgagttgaacataagtgcg

SEQ ID NO: 50 - DNA, 46 bp, Artificial Sequence
  mm-HL4X-Ager-1211
  source (1)..(46): sequence is synthesized
ttagtaggcgaacttacgtcgttatgtccgcttcctctgactgatt

SEQ ID NO: 51 - DNA, 46 bp, Artificial Sequence
  mm-HR4X-Ager-1211
  source (1)..(46): sequence is synthesized
ggcaccattctctggcatcttatacgtcgagttgaacataagtgcg

SEQ ID NO: 52 - DNA, 45 bp, Artificial Sequence
  mm-HL4X-Ager-976
  source (1)..(45): sequence is synthesized
ttagtaggcgaacttacgtcgttatgtgacaggagggctttcctg

SEQ ID NO: 53 - DNA, 47 bp, Artificial Sequence
  mm-HR4X-Ager-976
  source (1)..(47): sequence is synthesized
ggtttctgtgaccctgatgcttatacgtcgagttgaacataagtgcg

SEQ ID NO: 54 - DNA, 48 bp, Artificial Sequence
  mm-HL3X-Ftl1-808
  source (1)..(48): sequence is synthesized
tattcgttcgaacttacgtcgttatgtcctagtcgtgcttgagagtga

SEQ ID NO: 55 - DNA, 47 bp, Artificial Sequence
  mm-HR3X-Ftl1-808
  source (1)..(47): sequence is synthesized
ccttggaaggtacagaggccttatacgtcgagttgaccgacgtattg

SEQ ID NO: 56 - DNA, 46 bp, Artificial Sequence
  mm-HL3X-Ftl1-515
  source (1)..(46): sequence is synthesized
tattcgttcgaacttacgtcgttatgttgagatggcttctgcacat

SEQ ID NO: 57 - DNA, 47 bp, Artificial Sequence
  mm-HR3X-Ftl1-515
  source (1)..(47): sequence is synthesized
tgggttttaccccattcatcttatacgtcgagttgaccgacgtattg

SEQ ID NO: 58 - DNA, 45 bp, Artificial Sequence
  mm-HL3X-Ftl1-152
  source (1)..(45): sequence is synthesized
tattcgttcgaacttacgtcgttatgttgtaaaggcggctggaag

SEQ ID NO: 59 - DNA, 46 bp, Artificial Sequence
  mm-HR3X-Ftl1-152
  source (1)..(46): sequence is synthesized
aggctgcgactggagagacttatacgtcgagttgaccgacgtattg

SEQ ID NO: 60 - DNA, 44 bp, Artificial Sequence
  mm-HL3X-Ftl1-204
  source (1)..(44): sequence is synthesized
tattcgttcgaacttacgtcgttatgtaggagctgaaggcagcg

SEQ ID NO: 61 - DNA, 45 bp, Artificial Sequence
  mm-HR3X-Ftl1-204
  source (1)..(45): sequence is synthesized
gacggtgcagactggtccttatacgtcgagttgaccgacgtattg

SEQ ID NO: 62 - DNA, 44 bp, Artificial Sequence
  mm-HL3X-Ftl1-391
  source (1)..(44): sequence is synthesized
tattcgttcgaacttacgtcgttatgtccagagccacgtcatcc

SEQ ID NO: 63 - DNA, 45 bp, Artificial Sequence
  mm-HR3X-Ftl1-391
  source (1)..(45): sequence is synthesized
aagaagtggcctacgcccttatacgtcgagttgaccgacgtattg

SEQ ID NO: 64 - DNA, 43 bp, Artificial Sequence
  mm-HL5X-Scgb1a1-229
  source (1)..(43): sequence is synthesized
tagcgctaacaacttacgtcgttatgtcttcagctgggtgccc

SEQ ID NO: 65 - DNA, 46 bp, Artificial Sequence
  mm-HR5X-Scgb1a1-229
  source (1)..(46): sequence is synthesized
gggagggtatccaccagtcttatacgtcgagttgaacgtcgtaaca

SEQ ID NO: 66 - DNA, 48 bp, Artificial Sequence
  mm-HL5X-Scgb1a1-367
  source (1)..(48): sequence is synthesized
tagcgctaacaacttacgtcgttatgtctgaaatccagtgagcttcag

SEQ ID NO: 67 - DNA, 53 bp, Artificial Sequence
  mm-HR5X-Scgb1a1-367
  source (1)..(53): sequence is synthesized
tgacaaggctttagcagtagaatatcttatacgtcgagttgaacgtcgtaaca

SEQ ID NO: 68 - DNA, 50 bp, Artificial Sequence
  mm-HL2X-Sftpc-783
  source (1)..(50): sequence is synthesized
tcgtacgtctaacttacgtcgttatgtttattcttttgtgataggatccc

SEQ ID NO: 69 - DNA, 47 bp, Artificial Sequence
  mm-HR2X-Sftpc-783
  source (1)..(47): sequence is synthesized
ttgttttccaatcaggctgcttatacgtcgagttgaagaacaacctg

SEQ ID NO: 70 - DNA, 43 bp, Artificial Sequence
  mm-HL2X-Sftpc-646
  source (1)..(43): sequence is synthesized
tcgtacgtctaacttacgtcgttatgtgcggtttctaccgacc

SEQ ID NO: 71 - DNA, 44 bp, Artificial Sequence
  mm-HR2X-Sftpc-646
  source (1)..(44): sequence is synthesized
ggtctttcctgtcccgcttatacgtcgagttgaagaacaacctg

SEQ ID NO: 72 - DNA, 46 bp, Artificial Sequence
  mm-HL6X-Actb-610
  source (1)..(46): sequence is synthesized
taggtcaggaaacttacgtcgttatgtcgtagatgggcacagtgtg

SEQ ID NO: 73 - DNA, 46 bp, Artificial Sequence
  mm-HR6X-Actb-610
  source (1)..(46): sequence is synthesized
gtgagggagagcatagcccttatacgtcgagttgaatagccaggtt

SEQ ID NO: 74 - DNA, 47 bp, Artificial Sequence
  mm-HL6X-Actb-1111
  source (1)..(47): sequence is synthesized
taggtcaggaaacttacgtcgttatgtcaggaggagcaatgatcttg

SEQ ID NO: 75 - DNA, 47 bp, Artificial Sequence
  mm-HR6X-Actb-1111
  source (1)..(47): sequence is synthesized
tccacacagagtacttgcgcttatacgtcgagttgaatagccaggtt

SEQ ID NO: 76 - DNA, 44 bp, Artificial Sequence
  mm-HL6X-Actb-1465
  source (1)..(44): sequence is synthesized
taggtcaggaaacttacgtcgttatgtccaaccaactgctgtcg

SEQ ID NO: 77 - DNA, 46 bp, Artificial Sequence
  mm-HR6X-Actb-1465
  source (1)..(46): sequence is synthesized
aactttgggggatgtttgcttatacgtcgagttgaatagccaggtt

SEQ ID NO: 78 - DNA, 47 bp, Artificial Sequence
  mm-HL6X-Actb-1771
  source (1)..(47): sequence is synthesized
taggtcaggaaacttacgtcgttatgtgggccattcagaaattaaaa

SEQ ID NO: 79 - DNA, 46 bp, Artificial Sequence
  mm-HR6X-Actb-1771
  source (1)..(46): sequence is synthesized
aaaagggaggcctcagaccttatacgtcgagttgaatagccaggtt

SEQ ID NO: 80 - DNA, 46 bp, Artificial Sequence
  mm-HL11X-Gapdh-360
  source (1)..(46): sequence is synthesized
tgttcgtgttaacttacgtcgttatgactggaacatgtagaccatg

SEQ ID NO: 81 - DNA, 49 bp, Artificial Sequence
  mm-HR11X-Gapdh-360
  source (1)..(49): sequence is synthesized
tttgccgtgagtggagtcatttatacgtcgagttgaatcttgattcggt

SEQ ID NO: 82 - DNA, 46 bp, Artificial Sequence
  mm-HL11X-Gapdh-632
  source (1)..(46): sequence is synthesized
tgttcgtgttaacttacgtcgttatgatttctcgtggttcacaccc

SEQ ID NO: 83 - DNA, 49 bp, Artificial Sequence
  mm-HR11X-Gapdh-632
  source (1)..(49): sequence is synthesized
aatcttgagtgagttgtcatttatacgtcgagttgaatcttgattcggt

SEQ ID NO: 84 - DNA, 46 bp, Artificial Sequence
  mm-HL11X-Gapdh-923
  source (1)..(46): sequence is synthesized
tgttcgtgttaacttacgtcgttatgaggaacacggaaggccatgc

SEQ ID NO: 85 - DNA, 49 bp, Artificial Sequence
  mm-HR11X-Gapdh-923
  source (1)..(49): sequence is synthesized
acgacggacacattgggggtttatacgtcgagttgaatcttgattcggt

SEQ ID NO: 86 - DNA, 46 bp, Artificial Sequence
  mm-HL11X-Gapdh-1155
  source (1)..(46): sequence is synthesized
tgttcgtgttaacttacgtcgttatgaccaggaaatgagcttgaca

SEQ ID NO: 87 - DNA, 49 bp, Artificial Sequence
  mm-HR11X-Gapdh-1155
  source (1)..(49): sequence is synthesized
gtagccgtattcattgtcatttatacgtcgagttgaatcttgattcggt

SEQ ID NO: 88 - DNA, 46 bp, Artificial Sequence
  mm-HL11X-Gapdh-1385
  source (1)..(46): sequence is synthesized
tgttcgtgttaacttacgtcgttatgattgatggtattcaagagag

SEQ ID NO: 89 - DNA, 49 bp, Artificial Sequence
  mm-HR11X-Gapdh-1385
  source (1)..(49): sequence is synthesized
ttgtgggtgcagcgaactttttatacgtcgagttgaatcttgattcggt

SEQ ID NO: 90 - DNA, 46 bp, Artificial Sequence
  mm-HL7X-Xist-1447
  source (1)..(46): sequence is synthesized
gtgtaattgtcacttacgtcgttatgttagacacattcaagagcat

SEQ ID NO: 91 - DNA, 46 bp, Artificial Sequence
  mm-HR7X-Xist-1447
  source (1)..(46): sequence is synthesized
tcacatgacttccgccatcttatacgtcgagttgatattcctcagg

SEQ ID NO: 92 - DNA, 46 bp, Artificial Sequence
  mm-HL7X-Xist-2130
  source (1)..(46): sequence is synthesized
gtgtaattgtcacttacgtcgttatgttgagatcagtgctggctaa

SEQ ID NO: 93 - DNA, 47 bp, Artificial Sequence
  mm-HR7X-Xist-2130
  source (1)..(47): sequence is synthesized
cagtaggcttagagaaccgcttatacgtcgagttgatattcctcagg

SEQ ID NO: 94 - DNA, 46 bp, Artificial Sequence
  mm-HL7X-Xist-8362
  source (1)..(46): sequence is synthesized
gtgtaattgtcacttacgtcgttatgtaagcttccttctcaggaag

SEQ ID NO: 95 - DNA, 47 bp, Artificial Sequence
  mm-HR7X-Xist-8362
  source (1)..(47): sequence is synthesized
aagaacggaaagaaaagtgcttatacgtcgagttgatattcctcagg

SEQ ID NO: 96 - DNA, 46 bp, Artificial Sequence
  mm-HL7X-Xist-10145
  source (1)..(46): sequence is synthesized
gtgtaattgtcacttacgtcgttatgttgatcacgctgaagaccca

SEQ ID NO: 97 - DNA, 47 bp, Artificial Sequence
  mm-HR7X-Xist-10145
  source (1)..(47): sequence is synthesized
cttttgtgtgttcacattgcttatacgtcgagttgatattcctcagg

SEQ ID NO: 98 - DNA, 46 bp, Artificial Sequence
  mm-HL7X-Xist-16942
  source (1)..(46): sequence is synthesized
gtgtaattgtcacttacgtcgttatgtttcgttcctccagttttct

SEQ ID NO: 99 - DNA, 47 bp, Artificial Sequence
  mm-HR7X-Xist-16942
  source (1)..(47): sequence is synthesized
caagttctttcaatttggtcttatacgtcgagttgatattcctcagg

SEQ ID NO: 100 - DNA, 46 bp, Artificial Sequence
  mm-HL12X-Lyz2-920
  source (1)..(46): sequence is synthesized
tcaagtatggtacttacgtcgttatgtcctgtgccctcagaaacct

SEQ ID NO: 101 - DNA, 48 bp, Artificial Sequence
  mm-HR12X-Lyz2-920
  source (1)..(48): sequence is synthesized
tcagttctcatccactgagacttatacgtcgagttgagatgtatcctg

SEQ ID NO: 102 - DNA, 46 bp, Artificial Sequence
  mm-HL12X-Lyz2-791
  source (1)..(46): sequence is synthesized
tcaagtatggtacttacgtcgttatgttgtatttttattcacacag

SEQ ID NO: 103 - DNA, 47 bp, Artificial Sequence
  mm-HR12X-Lyz2-791
  source (1)..(47): sequence is synthesized
tggtctcctataagcagttcttatacgtcgagttgagatgtatcctg

SEQ ID NO: 104 - DNA, 46 bp, Artificial Sequence
  mm-HL12X-Lyz2-607
  source (1)..(46): sequence is synthesized
tcaagtatggtacttacgtcgttatgtgaagcttgtgaggaggaag

SEQ ID NO: 105 - DNA, 47 bp, Artificial Sequence
  mm-HR12X-Lyz2-607
  source (1)..(47): sequence is synthesized
ttgcacaacacagcatcagcttatacgtcgagttgagatgtatcctg

SEQ ID NO: 106 - DNA, 46 bp, Artificial Sequence
  mm-HL12X-Lyz2-422
  source (1)..(46): sequence is synthesized
tcaagtatggtacttacgtcgttatgtcggttttgacagtgtgctc

SEQ ID NO: 107 - DNA, 47 bp, Artificial Sequence
  mm-HR12X-Lyz2-422
  source (1)..(47): sequence is synthesized
cgaatatactgggacagatcttatacgtcgagttgagatgtatcctg

SEQ ID NO: 108 - DNA, 46 bp, Artificial Sequence
  mm-HL12X-Lyz2-246
  source (1)..(46): sequence is synthesized
tcaagtatggtacttacgtcgttatgtattgatctgaaatatccca

SEQ ID NO: 109 - DNA, 47 bp, Artificial Sequence
  mm-HR12X-Lyz2-246
  source (1)..(47): sequence is synthesized
atcattacaccagtatcggcttatacgtcgagttgagatgtatcctg

SEQ ID NO: 110 - DNA, 46 bp, Artificial Sequence
  mm-HL12X-Lyz2-67
  source (1)..(46): sequence is synthesized
tcaagtatggtacttacgtcgttatgttggcctgagcagtgacaga

SEQ ID NO: 111 - DNA, 47 bp, Artificial Sequence
  mm-HR12X-Lyz2-67
  source (1)..(47): sequence is synthesized
actcacaacgttcatagaccttatacgtcgagttgagatgtatcctg

SEQ ID NO: 112 - DNA, 56 bp, Artificial Sequence
  TRN10+HLR+10 -Scgb1a1-248
  source (1)..(56): sequence is synthesized
ggtctcttgtgggagggtatccaccagtctcttcagctgggtgcccgcattttgca

SEQ ID NO: 113 - DNA, 60 bp, Artificial Sequence
  Control Scrambled oligo
  source (1)..(60): sequence is synthesized
tatcgtgtaatcagtctcccaaagactcggacaacataacgacgtaagttagacgtacga

SEQ ID NO: 114 - DNA, 11 bp, Artificial Sequence
  ImO-Y1-A488-2
  source (1)..(11): sequence is synthesized
tgagctagaat

SEQ ID NO: 115 - DNA, 45 bp, Artificial Sequence
  mm-HL3X-Axin2-2956
  source (1)..(45): sequence is synthesized
tattcgttcgaacttacgtcgttatgatttcgtggctgttgcgta

SEQ ID NO: 116 - DNA, 46 bp, Artificial Sequence
  mm-HR3X-Axin2-2956
  source (1)..(46): sequence is synthesized
tttcattttccttcagaatttatacgtcgagttgaccgacgtattg

SEQ ID NO: 117 - DNA, 45 bp, Artificial Sequence
  mm-HL3X-Axin2-2412
  source (1)..(45): sequence is synthesized
tattcgttcgaacttacgtcgttatgagctgtgccaaagtgttgg

SEQ ID NO: 118 - DNA, 46 bp, Artificial Sequence
  mm-HR3X-Axin2-2412
  source (1)..(46): sequence is synthesized
cctgcggcaggcttcctctttatacgtcgagttgaccgacgtattg

SEQ ID NO: 119 - DNA, 44 bp, Artificial Sequence
  mm-HL3X-Axin2-1878
  source (1)..(44): sequence is synthesized
tattcgttcgaacttacgtcgttatgagtggtggtgaacgtgct

SEQ ID NO: 120 - DNA, 46 bp, Artificial Sequence
  mm-HR3X-Axin2-1878
  source (1)..(46): sequence is synthesized
acagcgtggtggtggatgtttatacgtcgagttgaccgacgtattg

SEQ ID NO: 121 - DNA, 46 bp, Artificial Sequence
  mm-HL3X-Axin2-347
  source (1)..(46): sequence is synthesized
tattcgttcgaacttacgtcgttatgaacacggcgctactcatggt

SEQ ID NO: 122 - DNA, 47 bp, Artificial Sequence
  mm-HR3X-Axin2-347
  source (1)..(47): sequence is synthesized
gatctggaaggagagtcactttatacgtcgagttgaccgacgtattg

SEQ ID NO: 123 - DNA, 72 bp, Artificial Sequence
  HCR1-H1-A488
  source (1)..(72): sequence is synthesized
gaagcgaatatggtgagagttggaggtaggttgaggcacatttacagacctcaacctacctccaactctcac

SEQ ID NO: 124 - DNA, 72 bp, Artificial Sequence
  HCR1-H2-A488
  source (1)..(72): sequence is synthesized
cctcaacctacctccaactctcaccatattcgcttcgtgagagttggaggtaggttgaggtctgtaaatgtg

SEQ ID NO: 125 - DNA, 132 bp, Artificial Sequence
  HCR1-Axin2-621-A488
  source (1)..(132): sequence is synthesized
cctcaacctacctccaactctcaccatattcgcttctaaaacgtatccacacatttctccctctccaggaaagtccggaagaggtatgcacattttcacatttacagacctcaacctacctccaactctcac

SEQ ID NO: 126 - DNA, 72 bp, Artificial Sequence
  HCR2-H1-A647
  source (1)..(72): sequence is synthesized
ggcggtttactggatgattgatgaggatttacgaggagctcagtccatcctcgtaaatcctcatcaatcatc

SEQ ID NO: 127 - DNA, 72 bp, Artificial Sequence
  HCR2-H2-A647
  source (1)..(72): sequence is synthesized
cctcgtaaatcctcatcaatcatccagtaaaccgccgatgattgatgaggatttacgaggatggactgagct

SEQ ID NO: 128 - DNA, 133 bp, Artificial Sequence
  HCR2-Axin2-1159-A647
  source (1)..(133): sequence is synthesized
cctcgtaaatcctcatcaatcatccagtaaaccgccaaaaaaacctacgtgataaggattgactgggtcgcttctcttgaaggacctgaataaaaaagctcagtccatcctcgtaaatcctcatcaatcatcc

SEQ ID NO: 129 - DNA, 45 bp, Artificial Sequence
  HL4X-MLLT10-4613
  source (1)..(45): sequence is synthesized
ttagtaggcgaacttacgtcgttatgtgtcacatcaaaagcgtgg

SEQ ID NO: 130 - DNA, 47 bp, Artificial Sequence
  HR4X-MLLT10-4613
  source (1)..(47): sequence is synthesized
ggggaatcactctgcggtacttatacgtcgagttgaacataagtgcg

SEQ ID NO: 131 - DNA, 51 bp, Artificial Sequence
  HL4X-MLLT10-4532
  source (1)..(51): sequence is synthesized
ttagtaggcgaacttacgtcgttatgttttaatggtcagtaatgggaagtg

SEQ ID NO: 132 - DNA, 50 bp, Artificial Sequence
  HR4X-MLLT10-4532
  source (1)..(50): sequence is synthesized
tttcaggaactctagtggtgagcttatacgtcgagttgaacataagtgcg

SEQ ID NO: 133 - DNA, 45 bp, Artificial Sequence
  HL5X-MLLT10-4613
  source (1)..(45): sequence is synthesized
tagcgctaacaacttacgtcgttatgtgtcacatcaaaagcgtgg

SEQ ID NO: 134 - DNA, 49 bp, Artificial Sequence
  HL5X-MLLT10-4418
  source (1)..(49): sequence is synthesized
tagcgctaacaacttacgtcgttatgtgagctatctacagtggcacaga

SEQ ID NO: 135 - DNA, 44 bp, Artificial Sequence
  HL5X-MLLT10-4277
  source (1)..(44): sequence is synthesized
tagcgctaacaacttacgtcgttatgttgatcaagggcaaaccc

SEQ ID NO: 136 - DNA, 48 bp, Artificial Sequence
  HL5X-MLLT10-3629
  source (1)..(48): sequence is synthesized
tagcgctaacaacttacgtcgttatgttacaagaatgtccttgcagca

SEQ ID NO: 137 - DNA, 47 bp, Artificial Sequence
  HR5X-MLLT10-4613
  source (1)..(47): sequence is synthesized
ggggaatcactctgcggtacttatacgtcgagttgaacgtcgtaaca

SEQ ID NO: 138 - DNA, 51 bp, Artificial Sequence
  HR5X-MLLT10-4418
  source (1)..(51): sequence is synthesized
aaatacccaggtaatagttttgccttatacgtcgagttgaacgtcgtaaca

SEQ ID NO: 139 - DNA, 45 bp, Artificial Sequence
  HR5X-MLLT10-4277
  source (1)..(45): sequence is synthesized
gcagcacaatggaggcacttatacgtcgagttgaacgtcgtaaca

SEQ ID NO: 140 - DNA, 51 bp, Artificial Sequence
  HR5X-MLLT10-3629
  source (1)..(51): sequence is synthesized
gcaacaagaaaactaatcaaagccttatacgtcgagttgaacgtcgtaaca

SEQ ID NO: 141 - DNA, 51 bp, Artificial Sequence
  HL5X-MLLT10-4532
  source (1)..(51): sequence is synthesized
tagcgctaacaacttacgtcgttatgttttaatggtcagtaatgggaagtg

SEQ ID NO: 142 - DNA, 45 bp, Artificial Sequence
  HL5X-MLLT10-4338
  source (1)..(45): sequence is synthesized
tagcgctaacaacttacgtcgttatgtgctcggaggactgactgt

SEQ ID NO: 143 - DNA, 52 bp, Artificial Sequence
  HL5X-MLLT10-4177
  source (1)..(52): sequence is synthesized
tagcgctaacaacttacgtcgttatgtcacttaactcaagatttcttcaaaa

SEQ ID NO: 144 - DNA, 48 bp, Artificial Sequence
  HL5X-MLLT10-3579
  source (1)..(48): sequence is synthesized
tagcgctaacaacttacgtcgttatgttgttgcgtttcttcatagctg

SEQ ID NO: 145 - DNA, 46 bp, Artificial Sequence
  HL5X-MLLT10-2221
  source (1)..(46): sequence is synthesized
tagcgctaacaacttacgtcgttatgtcctacttgttgcaaatgcc

SEQ ID NO: 146 - DNA, 47 bp, Artificial Sequence
  HL5X-MLLT10-1312
  source (1)..(47): sequence is synthesized
tagcgctaacaacttacgtcgttatgtgaagatttcttccctttgcc

SEQ ID NO: 147 - DNA, 47 bp, Artificial Sequence
  HR5X-MLLT10-1312
  source (1)..(47): sequence is synthesized
ctttgacctgagctgtgagcttatacgtcgagttgaacgtcgtaaca

SEQ ID NO: 148 - DNA, 50 bp, Artificial Sequence
  HR5X-MLLT10-4532
  source (1)..(50): sequence is synthesized
tttcaggaactctagtggtgagcttatacgtcgagttgaacgtcgtaaca

SEQ ID NO: 149 - DNA, 46 bp, Artificial Sequence
  HR5X-MLLT10-4338
  source (1)..(46): sequence is synthesized
gtgacgggatgattcctgcttatacgtcgagttgaacgtcgtaaca

SEQ ID NO: 150 - DNA, 45 bp, Artificial Sequence
  HR5X-MLLT10-4177
  source (1)..(45): sequence is synthesized
ccccagcagcctcagaacttatacgtcgagttgaacgtcgtaaca

SEQ ID NO: 151 - DNA, 49 bp, Artificial Sequence
  HR5X-MLLT10-3579
  source (1)..(49): sequence is synthesized
cctttgttgtgcattgagtttcttatacgtcgagttgaacgtcgtaaca

SEQ ID NO: 152 - DNA, 45 bp, Artificial Sequence
  HR5X-MLLT10-2221
  source (1)..(45): sequence is synthesized
agctgagggagagagcgcttatacgtcgagttgaacgtcgtaaca

SEQ ID NO: 153 - DNA, 45 bp, Artificial Sequence
  HL2X-MLLT10-4613
  source (1)..(45): sequence is synthesized
tcgtacgtctaacttacgtcgttatgtgtcacatcaaaagcgtgg

SEQ ID NO: 154 - DNA, 49 bp, Artificial Sequence
  HL2X-MLLT10-4418
  source (1)..(49): sequence is synthesized
tcgtacgtctaacttacgtcgttatgtgagctatctacagtggcacaga

SEQ ID NO: 155 - DNA, 44 bp, Artificial Sequence
  HL2X-MLLT10-4277
  source (1)..(44): sequence is synthesized
tcgtacgtctaacttacgtcgttatgttgatcaagggcaaaccc

SEQ ID NO: 156 - DNA, 48 bp, Artificial Sequence
  HL2X-MLLT10-3629
  source (1)..(48): sequence is synthesized
tcgtacgtctaacttacgtcgttatgttacaagaatgtccttgcagca

SEQ ID NO: 157 - DNA, 47 bp, Artificial Sequence
  HL2X-MLLT10-2559
  source (1)..(47): sequence is synthesized
tcgtacgtctaacttacgtcgttatgtccaaaagctgttctatgctg

SEQ ID NO: 158 - DNA, 46 bp, Artificial Sequence
  HL2X-MLLT10-1475
  source (1)..(46): sequence is synthesized
tcgtacgtctaacttacgtcgttatgtcaggaaatcctggggagac

SEQ ID NO: 159 - DNA, 47 bp, Artificial Sequence
  HR2X-MLLT10-4613
  source (1)..(47): sequence is synthesized
ggggaatcactctgcggtacttatacgtcgagttgaagaacaacctg

SEQ ID NO: 160 - DNA, 51 bp, Artificial Sequence
  HR2X-MLLT10-4418
  source (1)..(51): sequence is synthesized
aaatacccaggtaatagttttgccttatacgtcgagttgaagaacaacctg

SEQ ID NO: 161 - DNA, 45 bp, Artificial Sequence
  HR2X-MLLT10-4277
  source (1)..(45): sequence is synthesized
gcagcacaatggaggcacttatacgtcgagttgaagaacaacctg

SEQ ID NO: 162 - DNA, 51 bp, Artificial Sequence
  HR2X-MLLT10-3629
  source (1)..(51): sequence is synthesized
gcaacaagaaaactaatcaaagccttatacgtcgagttgaagaacaacctg

SEQ ID NO: 163 - DNA, 47 bp, Artificial Sequence
  HR2X-MLLT10-2559
  source (1)..(47): sequence is synthesized
gtccttcactccactgcctcttatacgtcgagttgaagaacaacctg

SEQ ID NO: 164 - DNA, 49 bp, Artificial Sequence
  HR2X-MLLT10-1475
  source (1)..(49): sequence is synthesized
cgcagatctgagtctgtaaagcttatacgtcgagttgaagaacaacctg

SEQ ID NO: 165 - DNA, 45 bp, Artificial Sequence
  HL6X-ETV5-1840
  source (1)..(45): sequence is synthesized
taggtcaggaaacttacgtcgttatgttgtttgcctgaatgggaa

SEQ ID NO: 166 - DNA, 47 bp, Artificial Sequence
  HR6X-ETV5-1840
  source (1)..(47): sequence is synthesized
cacaaacaaaaccactgcccttatacgtcgagttgaatagccaggtt

SEQ ID NO: 167 - DNA, 50 bp, Artificial Sequence
  HL6X-ETV5-1911
  source (1)..(50): sequence is synthesized
taggtcaggaaacttacgtcgttatgttgggttctccagataatactcaa

SEQ ID NO: 168 - DNA, 46 bp, Artificial Sequence
  HR6X-ETV5-1911
  source (1)..(46): sequence is synthesized
gtgccaatccagagacagcttatacgtcgagttgaatagccaggtt

SEQ ID NO: 169 - DNA, 44 bp, Artificial Sequence
  HL2X-ETV5-1424
  source (1)..(44): sequence is synthesized
tcgtacgtctaacttacgtcgttatgtcgacctgtccaggcaat

SEQ ID NO: 170 - DNA, 47 bp, Artificial Sequence
  HR2X-ETV5-1424
  source (1)..(47): sequence is synthesized
atcagcttgaactccatgccttatacgtcgagttgaagaacaacctg

SEQ ID NO: 171 - DNA, 44 bp, Artificial Sequence
  HL2X-ETV5-1709
  source (1)..(44): sequence is synthesized
tcgtacgtctaacttacgtcgttatgttcaaagtgggtcagcgg

SEQ ID NO: 172 - DNA, 45 bp, Artificial Sequence
  HR2X-ETV5-1709
  source (1)..(45): sequence is synthesized
gaggtaagcggggctgtcttatacgtcgagttgaagaacaacctg

SEQ ID NO: 173 - DNA, 46 bp, Artificial Sequence
  HL2X-ETV5-3344
  source (1)..(46): sequence is synthesized
tcgtacgtctaacttacgtcgttatgtgcctccttcacatacgagg

SEQ ID NO: 174 - DNA, 47 bp, Artificial Sequence
  HR2X-ETV5-3344
  source (1)..(47): sequence is synthesized
cacactcacggagtcagcacttatacgtcgagttgaagaacaacctg

SEQ ID NO: 175 - DNA, 47 bp, Artificial Sequence
  HL2X-ETV5-3453
  source (1)..(47): sequence is synthesized
tcgtacgtctaacttacgtcgttatgtcttcacaaatgtgctgttgg

SEQ ID NO: 176 - DNA, 45 bp, Artificial Sequence
  HR2X-ETV5-3453
  source (1)..(45): sequence is synthesized
cccttatccctgcgaaccttatacgtcgagttgaagaacaacctg

SEQ ID NO: 177 - DNA, 45 bp, Artificial Sequence
  HL2X-ETV5-1821
  source (1)..(45): sequence is synthesized
tcgtacgtctaacttacgtcgttatgttgtttgcctgaatgggaa

SEQ ID NO: 178 - DNA, 47 bp, Artificial Sequence
  HR2X-ETV5-1840
  source (1)..(47): sequence is synthesized
cacaaacaaaaccactgcccttatacgtcgagttgaagaacaacctg

SEQ ID NO: 179 - DNA, 47 bp, Artificial Sequence
  HL5X-ETV5-3453
  source (1)..(47): sequence is synthesized
tagcgctaacaacttacgtcgttatgtcttcacaaatgtgctgttgg

SEQ ID NO: 180 - DNA, 45 bp, Artificial Sequence
  HR5X-ETV5-3453
  source (1)..(45): sequence is synthesized
cccttatccctgcgaaccttatacgtcgagttgaacgtcgtaaca

SEQ ID NO: 181 - DNA, 48 bp, Artificial Sequence
  HL5X-ETV5-222
  source (1)..(48): sequence is synthesized
tagcgctaacaacttacgtcgttatgtttgagaggtttcagcattgag

SEQ ID NO: 182 - DNA, 50 bp, Artificial Sequence
  HR5X-ETV5-222
  source (1)..(50): sequence is synthesized
gctttcagcgtctctaataccacttatacgtcgagttgaacgtcgtaaca

SEQ ID NO: 183 - DNA, 45 bp, Artificial Sequence
  HL5X-ETV5-1132
  source (1)..(45): sequence is synthesized
tagcgctaacaacttacgtcgttatgtcctgcttgattcccattg

SEQ ID NO: 184 - DNA, 45 bp, Artificial Sequence
  HR5X-ETV5-1132
  source (1)..(45): sequence is synthesized
acgcagtaatcccgaggcttatacgtcgagttgaacgtcgtaaca

SEQ ID NO: 185 - DNA, 45 bp, Artificial Sequence
  HL5X-ETV5-1991
  source (1)..(45): sequence is synthesized
tagcgctaacaacttacgtcgttatgttttggtggttttctgccc

SEQ ID NO: 186 - DNA, 46 bp, Artificial Sequence
  HR5X-ETV5-1991
  source (1)..(46): sequence is synthesized
aagagttgaggcactggccttatacgtcgagttgaacgtcgtaaca

SEQ ID NO: 187 - DNA, 49 bp, Artificial Sequence
  HL5X-ETV5-2091
  source (1)..(49): sequence is synthesized
tagcgctaacaacttacgtcgttatgtgaacaagatattgctttgcatg

SEQ ID NO: 188 - DNA, 47 bp, Artificial Sequence
  HR5X-ETV5-2091
  source (1)..(47): sequence is synthesized
ccgttttgcgggtactaaccttatacgtcgagttgaacgtcgtaaca

SEQ ID NO: 189 - DNA, 44 bp, Artificial Sequence
  HL5X-ETV5-2423
  source (1)..(44): sequence is synthesized
tagcgctaacaacttacgtcgttatgtacttggcgaccacatgg

SEQ ID NO: 190 - DNA, 46 bp, Artificial Sequence
  HR5X-ETV5-2423
  source (1)..(46): sequence is synthesized
agagaaagggggcttgctcttatacgtcgagttgaacgtcgtaaca

SEQ ID NO: 191 - DNA, 47 bp, Artificial Sequence
  HL5X-ETV5-2508
  source (1)..(47): sequence is synthesized
tagcgctaacaacttacgtcgttatgtgtcaaaagtgttaatcgccc

SEQ ID NO: 192 - DNA, 50 bp, Artificial Sequence
  HR5X-ETV5-2508
  source (1)..(50): sequence is synthesized
caaatcagggcaaaacaaaatacttatacgtcgagttgaacgtcgtaaca

SEQ ID NO: 193 - DNA, 46 bp, Artificial Sequence
  HL5X-ETV5-2672
  source (1)..(46): sequence is synthesized
tagcgctaacaacttacgtcgttatgtggctaaaggacacagtgca

SEQ ID NO: 194 - DNA, 45 bp, Artificial Sequence
  HR5X-ETV5-2672
  source (1)..(45): sequence is synthesized
cataagttggcacggcccttatacgtcgagttgaacgtcgtaaca

SEQ ID NO: 195 - DNA, 44 bp, Artificial Sequence
  HL5X-ETV5-2837
  source (1)..(44): sequence is synthesized
tagcgctaacaacttacgtcgttatgtgccctgcacacttcaaa

SEQ ID NO: 196 - DNA, 49 bp, Artificial Sequence
  HR5X-ETV5-2837
  source (1)..(49): sequence is synthesized
gaatctaagcttgtgtccaggcttatacgtcgagttgaacgtcgtaaca

SEQ ID NO: 197 - DNA, 50 bp, Artificial Sequence
  HL5X-ETV5-1887
  source (1)..(50): sequence is synthesized
tagcgctaacaacttacgtcgttatgttgggttctccagataatactcaa

SEQ ID NO: 198 - DNA, 46 bp, Artificial Sequence
  HR5X-ETV5-1911
  source (1)..(46): sequence is synthesized
gtgccaatccagagacagcttatacgtcgagttgaacgtcgtaaca

SEQ ID NO: 199 - DNA, 46 bp, Artificial Sequence
  HL3X-CASP9-1542
  source (1)..(46): sequence is synthesized
tattcgttcgaacttacgtcgttatgtcttcctccactgttcagca

SEQ ID NO: 200 - DNA, 45 bp, Artificial Sequence
  HR3X-CASP9-1542
  source (1)..(45): sequence is synthesized
acggcattcatctgtcccttatacgtcgagttgaccgacgtattg

SEQ ID NO: 201 - DNA, 45 bp, Artificial Sequence
  HL3X-CASP9-972
  source (1)..(45): sequence is synthesized
tattcgttcgaacttacgtcgttatgtttctgctccccaccacag

SEQ ID NO: 202 - DNA, 46 bp, Artificial Sequence
  HR3X-CASP9-972
  source (1)..(46): sequence is synthesized
ccacctcaaacccatggtcttatacgtcgagttgaccgacgtattg

SEQ ID NO: 203 - DNA, 47 bp, Artificial Sequence
  HL5X-CASP9-1850
  source (1)..(47): sequence is synthesized
tagcgctaacaacttacgtcgttatgtacagaccagtggttgtcagg

SEQ ID NO: 204 - DNA, 48 bp, Artificial Sequence
  HR5X-CASP9-1850
  source (1)..(48): sequence is synthesized
ttgtgtgtagccaaaaatcccttatacgtcgagttgaacgtcgtaaca

SEQ ID NO: 205 - DNA, 47 bp, Artificial Sequence
  HL5X-CASP9-1789
  source (1)..(47): sequence is synthesized
tagcgctaacaacttacgtcgttatgtgagaacctttttgtttggca

SEQ ID NO: 206 - DNA, 48 bp, Artificial Sequence
  HR5X-CASP9-1789
  source (1)..(48): sequence is synthesized
caggacatatttggcaacaccttatacgtcgagttgaacgtcgtaaca

SEQ ID NO: 207 - DNA, 45 bp, Artificial Sequence
  HL5X-CASP9-1695
  source (1)..(45): sequence is synthesized
tagcgctaacaacttacgtcgttatgtcacagcaaagggtgacct

SEQ ID NO: 208 - DNA, 47 bp, Artificial Sequence
  HR5X-CASP9-1695
  source (1)..(47): sequence is synthesized
ccacaatgtacaggacagccttatacgtcgagttgaacgtcgtaaca

SEQ ID NO: 209 - DNA, 49 bp, Artificial Sequence
  HL5X-CASP9-1292
  source (1)..(49): sequence is synthesized
tagcgctaacaacttacgtcgttatgtgtttataaatccctttcaccga

SEQ ID NO: 210 - DNA, 49 bp, Artificial Sequence
  HR5X-CASP9-1292
  source (1)..(49): sequence is synthesized
gaaattaaagcaaccaggcatcttatacgtcgagttgaacgtcgtaaca

SEQ ID NO: 211 - DNA, 46 bp, Artificial Sequence
  HL5X-CASP9-1211
  source (1)..(46): sequence is synthesized
tagcgctaacaacttacgtcgttatgtcaaagatgtcgtccagggt

SEQ ID NO: 212 - DNA, 46 bp, Artificial Sequence
  HR5X-CASP9-1211
  source (1)..(46): sequence is synthesized
ttcagagtgagcccactgcttatacgtcgagttgaacgtcgtaaca

SEQ ID NO: 213 - DNA, 46 bp, Artificial Sequence
  HL5X-CASP9-1096
  source (1)..(46): sequence is synthesized
tagcgctaacaacttacgtcgttatgtagatatggcgtccagctgg

SEQ ID NO: 214 - DNA, 45 bp, Artificial Sequence
  HR5X-CASP9-1096
  source (1)..(45): sequence is synthesized
cactgggtgtgggcaaacttatacgtcgagttgaacgtcgtaaca

SEQ ID NO: 215 - DNA, 44 bp, Artificial Sequence
  HL5X-CASP9-881
  source (1)..(44): sequence is synthesized
tagcgctaacaacttacgtcgttatgttctcgaccgacacaggg

SEQ ID NO: 216 - DNA, 49 bp, Artificial Sequence
  HR5X-CASP9-881
  source (1)..(49): sequence is synthesized
cccattgaagatgttcacaatcttatacgtcgagttgaacgtcgtaaca

SEQ ID NO: 217 - DNA, 44 bp, Artificial Sequence
  HL5X-CASP9-701
  source (1)..(44): sequence is synthesized
tagcgctaacaacttacgtcgttatgtccaccatgaaatgcagc

SEQ ID NO: 218 - DNA, 46 bp, Artificial Sequence
  HR5X-CASP9-701
  source (1)..(46): sequence is synthesized
agtcaggtcgcccttcaccttatacgtcgagttgaacgtcgtaaca

SEQ ID NO: 219 - DNA, 46 bp, Artificial Sequence
  HL5X-CASP9-341
  source (1)..(46): sequence is synthesized
tagcgctaacaacttacgtcgttatgtggcctgtgtcctctaagca

SEQ ID NO: 220 - DNA, 46 bp, Artificial Sequence
  HR5X-CASP9-341
  source (1)..(46): sequence is synthesized
aaacgaagccagcatgtccttatacgtcgagttgaacgtcgtaaca

SEQ ID NO: 221 - DNA, 47 bp, Artificial Sequence
  HL5X-CASP9-284
  source (1)..(47): sequence is synthesized
tagcgctaacaacttacgtcgttatgtccagatctatgatcagctgc

SEQ ID NO: 222 - DNA, 46 bp, Artificial Sequence
  HR5X-CASP9-284
  source (1)..(46): sequence is synthesized
agcctgactccctcgagtcttatacgtcgagttgaacgtcgtaaca

SEQ ID NO: 223 - DNA, 44 bp, Artificial Sequence
  HL2X-CASP9-1602
  source (1)..(44): sequence is synthesized
tcgtacgtctaacttacgtcgttatgtagccctggaccagccac

SEQ ID NO: 224 - DNA, 48 bp, Artificial Sequence
  HR2X-CASP9-1602
  source (1)..(48): sequence is synthesized
ggatcatgggacacaagtcacttatacgtcgagttgaagaacaacctg

SEQ ID NO: 225 - DNA, 46 bp, Artificial Sequence
  HL2X-CASP9-1542
  source (1)..(46): sequence is synthesized
tcgtacgtctaacttacgtcgttatgtcttcctccactgttcagca

SEQ ID NO: 226 - DNA, 45 bp, Artificial Sequence
  HR2X-CASP9-1542
  source (1)..(45): sequence is synthesized
acggcattcatctgtcccttatacgtcgagttgaagaacaacctg

SEQ ID NO: 227 - DNA, 47 bp, Artificial Sequence
  HL2X-CASP9-1499
  source (1)..(47): sequence is synthesized
tcgtacgtctaacttacgtcgttatgtaagagcctgtctgtcactgg

SEQ ID NO: 228 - DNA, 47 bp, Artificial Sequence
  HR2X-CASP9-1499
  source (1)..(47): sequence is synthesized
gtcgtcaatctggaagctgcttatacgtcgagttgaagaacaacctg

SEQ ID NO: 229 - DNA, 45 bp, Artificial Sequence
  HL2X-CASP9-1417
  source (1)..(45): sequence is synthesized
tcgtacgtctaacttacgtcgttatgtcagcctctttcaggcctg

SEQ ID NO: 230 - DNA, 45 bp, Artificial Sequence
  HR2X-CASP9-1417
  source (1)..(45): sequence is synthesized
tgcaggaaagtccaggccttatacgtcgagttgaagaacaacctg

SEQ ID NO: 231 - DNA, 44 bp, Artificial Sequence
  HL2X-CASP9-1024
  source (1)..(44): sequence is synthesized
tcgtacgtctaacttacgtcgttatgtgccaggggactcgtctt

SEQ ID NO: 232 - DNA, 45 bp, Artificial Sequence
  HR2X-CASP9-1024
  source (1)..(45): sequence is synthesized
catctggctcggggttacttatacgtcgagttgaagaacaacctg

SEQ ID NO: 233 - DNA, 45 bp, Artificial Sequence
  HL2X-CASP9-972
  source (1)..(45): sequence is synthesized
tcgtacgtctaacttacgtcgttatgtttctgctccccaccacag

SEQ ID NO: 234 - DNA, 46 bp, Artificial Sequence
  HR2X-CASP9-972
  source (1)..(46): sequence is synthesized
ccacctcaaacccatggtcttatacgtcgagttgaagaacaacctg

SEQ ID NO: 235 - DNA, 46 bp, Artificial Sequence
  HL2X-CASP9-543
  source (1)..(46): sequence is synthesized
tcgtacgtctaacttacgtcgttatgtgcatttcccctcaaactct

SEQ ID NO: 236 - DNA, 48 bp, Artificial Sequence
  HR2X-CASP9-543
  source (1)..(48): sequence is synthesized
gctcaggatgtaagccaaatcttatacgtcgagttgaagaacaacctg

SEQ ID NO: 237 - DNA, 45 bp, Artificial Sequence
  HL2X-CASP9-475
  source (1)..(45): sequence is synthesized
tcgtacgtctaacttacgtcgttatgtgggtgtttccggtctgag

SEQ ID NO: 238 - DNA, 48 bp, Artificial Sequence
  HR2X-CASP9-475
  source (1)..(48): sequence is synthesized
cagaaccaatgtccactggtcttatacgtcgagttgaagaacaacctg

SEQ ID NO: 239 - DNA, 46 bp, Artificial Sequence
  HL2X-CASP9-395
  source (1)..(46): sequence is synthesized
tcgtacgtctaacttacgtcgttatgttcgacaactttgctgcttg

SEQ ID NO: 240 - DNA, 49 bp, Artificial Sequence
  HR2X-CASP9-395
  source (1)..(49): sequence is synthesized
ggtaaggttttctagggttggcttatacgtcgagttgaagaacaacctg

SEQ ID NO: 241 - DNA, 44 bp, Artificial Sequence
  HL2X-CASP9-82
  source (1)..(44): sequence is synthesized
tcgtacgtctaacttacgtcgttatgtaagactccaggccgcct

SEQ ID NO: 242 - DNA, 45 bp, Artificial Sequence
  HR2X-CASP9-82
  source (1)..(45): sequence is synthesized
ccatggcgagtagccaacttatacgtcgagttgaagaacaacctg

SEQ ID NO: 243 - DNA, 48 bp, Artificial Sequence
  HL2X-GAPDH-1085
  source (1)..(48): sequence is synthesized
tcgtacgtctaacttacgtcgttatgtacttctcatggttcacaccca

SEQ ID NO: 244 - DNA, 49 bp, Artificial Sequence
  HR2X-GAPDH-1107
  source (1)..(49): sequence is synthesized
gatgatcttgaggctgttgtcattatacgtcgagttgaagaacaacctg

SEQ ID NO: 245 - DNA, 46 bp, Artificial Sequence
  HL2X-GAPDH-1329
  source (1)..(46): sequence is synthesized
tcgtacgtctaacttacgtcgttatggatgaccttgcccacagcct

SEQ ID NO: 246 - DNA, 47 bp, Artificial Sequence
  HR2X-GAPDH-1349
  source (1)..(47): sequence is synthesized
agcttcccgttcagctcaggttatacgtcgagttgaagaacaacctg

SEQ ID NO: 247 - DNA, 46 bp, Artificial Sequence
  HL2X-GAPDH-1757
  source (1)..(46): sequence is synthesized
tcgtacgtctaacttacgtcgttatggactgagtgtggcagggact

SEQ ID NO: 248 - DNA, 47 bp, Artificial Sequence
  HR2X-GAPDH-1777
  source (1)..(47): sequence is synthesized
ggagattcagtgtggtggggttatacgtcgagttgaagaacaacctg

SEQ ID NO: 249 - DNA, 44 bp, Artificial Sequence
  HL2X-GAPDH-1334
  source (1)..(44): sequence is synthesized
tcgtacgtctaacttacgtcgttatgtcagggatgaccttgccc

SEQ ID NO: 250 - DNA, 46 bp, Artificial Sequence
  HR2X-GAPDH-1353
  source (1)..(46): sequence is synthesized
agtgagcttcccgttcagcttatacgtcgagttgaagaacaacctg

SEQ ID NO: 251 - DNA, 45 bp, Artificial Sequence
  HL5X-GAPDH-1755
  source (1)..(45): sequence is synthesized
tagcgctaacaacttacgtcgttatgtgagtgtggcagggactcc

SEQ ID NO: 252 - DNA, 46 bp, Artificial Sequence
  HL5X-GAPDH-1715
  source (1)..(46): sequence is synthesized
tagcgctaacaacttacgtcgttatgttcctcttgtgctcttgctg

SEQ ID NO: 253 - DNA, 46 bp, Artificial Sequence
  HL5X-GAPDH-1634
  source (1)..(46): sequence is synthesized
tagcgctaacaacttacgtcgttatgtgttgctgtagccaaattcg

SEQ ID NO: 254 - DNA, 45 bp, Artificial Sequence
  HL5X-GAPDH-1500
  source (1)..(45): sequence is synthesized
tagcgctaacaacttacgtcgttatgtcagtgtagcccaggatgc

SEQ ID NO: 255 - DNA, 44 bp, Artificial Sequence
  HL5X-GAPDH-1390
  source (1)..(44): sequence is synthesized
tagcgctaacaacttacgtcgttatgtgacacgttggcagtggg

SEQ ID NO: 256 - DNA, 44 bp, Artificial Sequence
  HL5X-GAPDH-1311
  source (1)..(44): sequence is synthesized
tagcgctaacaacttacgtcgttatgttggcagcgccagtagag

SEQ ID NO: 257 - DNA, 44 bp, Artificial Sequence
  HL5X-GAPDH-1224
  source (1)..(44): sequence is synthesized
tagcgctaacaacttacgtcgttatgttctgggtggcagtgatg

SEQ ID NO: 258 - DNA, 44 bp, Artificial Sequence
  HL5X-GAPDH-984
  source (1)..(44): sequence is synthesized
tagcgctaacaacttacgtcgttatgtccatggtggtgaagacg

SEQ ID NO: 259 - DNA, 45 bp, Artificial Sequence
  HL5X-GAPDH-903
  source (1)..(45): sequence is synthesized
tagcgctaacaacttacgtcgttatgtcctggaagatggtgatgg

SEQ ID NO: 260 - DNA, 46 bp, Artificial Sequence
  HR5X-GAPDH-1755
  source (1)..(46): sequence is synthesized
attcagtgtggtgggggacttatacgtcgagttgaacgtcgtaaca

SEQ ID NO: 261 - DNA, 47 bp, Artificial Sequence
  HR5X-GAPDH-1715
  source (1)..(47): sequence is synthesized
cagcagtgagggtctctctcttatacgtcgagttgaacgtcgtaaca

SEQ ID NO: 262 - DNA, 45 bp, Artificial Sequence
  HR5X-GAPDH-1634
  source (1)..(45): sequence is synthesized
ccatgaggtccaccacccttatacgtcgagttgaacgtcgtaaca

SEQ ID NO: 263 - DNA, 46 bp, Artificial Sequence
  HR5X-GAPDH-1500
  source (1)..(46): sequence is synthesized
agaggagaccacctggtgcttatacgtcgagttgaacgtcgtaaca

SEQ ID NO: 264 - DNA, 45 bp, Artificial Sequence
  HR5X-GAPDH-1390
  source (1)..(45): sequence is synthesized
gcaggtcaggtccaccacttatacgtcgagttgaacgtcgtaaca

SEQ ID NO: 265 - DNA, 45 bp, Artificial Sequence
  HR5X-GAPDH-1311
  source (1)..(45): sequence is synthesized
atgaccttgcccacagccttatacgtcgagttgaacgtcgtaaca

SEQ ID NO: 266 - DNA, 45 bp, Artificial Sequence
  HR5X-GAPDH-1224
  source (1)..(45): sequence is synthesized
gaggggccatccacagtcttatacgtcgagttgaacgtcgtaaca

SEQ ID NO: 267 - DNA, 45 bp, Artificial Sequence
  HR5X-GAPDH-984
  source (1)..(45): sequence is synthesized
aaatgagccccagccttcttatacgtcgagttgaacgtcgtaaca

SEQ ID NO: 268 - DNA, 46 bp, Artificial Sequence
  HR5X-GAPDH-903
  source (1)..(46): sequence is synthesized
gattttggagggatctcgcttatacgtcgagttgaacgtcgtaaca

SEQ ID NO: 269 - DNA, 42 bp, Artificial Sequence
  HL5X1-GAPDH-1757
  source (1)..(42): sequence is synthesized
tagcgctaacattaagacgcttgactgagtgtggcagggact

SEQ ID NO: 270 - DNA, 45 bp, Artificial Sequence
  HR5X1-GAPDH-1777
  source (1)..(45): sequence is synthesized
ggagattcagtgtggtggggaaatatgacagaacacgtcgtaaca

SEQ ID NO: 271 - DNA, 46 bp, Artificial Sequence
  HL5X-GAPDH-1757
  source (1)..(46): sequence is synthesized
tagcgctaacaacttacgtcgttatggactgagtgtggcagggact

SEQ ID NO: 272 - DNA, 47 bp, Artificial Sequence
  HR5X-GAPDH-1777
  source (1)..(47): sequence is synthesized
ggagattcagtgtggtggggttatacgtcgagttgaacgtcgtaaca

SEQ ID NO: 273 - DNA, 47 bp, Artificial Sequence
  HL4X-GAPDH-1084
  source (1)..(47): sequence is synthesized
ttagtaggcgaacttacgtcgttatgacttctcatggttcacaccca

SEQ ID NO: 274 - DNA, 50 bp, Artificial Sequence
  HR4X-GAPDH-1107
  source (1)..(50): sequence is synthesized
gatgatcttgaggctgttgtcatttatacgtcgagttgaacataagtgcg

SEQ ID NO: 275 - DNA, 46 bp, Artificial Sequence
  HL4X-GAPDH-1757
  source (1)..(46): sequence is synthesized
ttagtaggcgaacttacgtcgttatggactgagtgtggcagggact

SEQ ID NO: 276 - DNA, 47 bp, Artificial Sequence
  HR4X-GAPDH-1777
  source (1)..(47): sequence is synthesized
ggagattcagtgtggtggggttatacgtcgagttgaacataagtgcg

SEQ ID NO: 277 - DNA, 42 bp, Artificial Sequence
  HL3X1-GAPDH-1757
  source (1)..(42): sequence is synthesized
tattcgttcgattaagacgcttgactgagtgtggcagggact

SEQ ID NO: 278 - DNA, 45 bp, Artificial Sequence
  HR3X1-GAPDH-1777
  source (1)..(45): sequence is synthesized
ggagattcagtgtggtggggaaatatgacagaacccgacgtattg

SEQ ID NO: 279 - DNA, 46 bp, Artificial Sequence
  HL3X-GAPDH-1757
  source (1)..(46): sequence is synthesized
tattcgttcgaacttacgtcgttatggactgagtgtggcagggact

SEQ ID NO: 280 - DNA, 47 bp, Artificial Sequence
  HR3X-GAPDH-1777
  source (1)..(47): sequence is synthesized
ggagattcagtgtggtggggttatacgtcgagttgaccgacgtattg

SEQ ID NO: 281 - DNA, 46 bp, Artificial Sequence
  HL5X-ACTB-1652
  source (1)..(46): sequence is synthesized
tagcgctaacaacttacgtcgttatgtgggccattctccttagaga

SEQ ID NO: 282 - DNA, 45 bp, Artificial Sequence
  HL5X-ACTB-1490
  source (1)..(45): sequence is synthesized
tagcgctaacaacttacgtcgttatgtgctgtcaccttcaccgtt

SEQ ID NO: 283 - DNA, 46 bp, Artificial Sequence
  HL5X-ACTB-1389
  source (1)..(46): sequence is synthesized
tagcgctaacaacttacgtcgttatgtcatcttgttttctgcgcaa

SEQ ID NO: 284 - DNA, 46 bp, Artificial Sequence
  HL5X-ACTB-1272
  source (1)..(46): sequence is synthesized
tagcgctaacaacttacgtcgttatgtgcttgctgatccacatctg

SEQ ID NO: 285 - DNA, 46 bp, Artificial Sequence
  HL5X-ACTB-1176
  source (1)..(46): sequence is synthesized
tagcgctaacaacttacgtcgttatgttgatcttcattgtgctggg

SEQ ID NO: 286 - DNA, 45 bp, Artificial Sequence
  HL5X-ACTB-908
  source (1)..(45): sequence is synthesized
tagcgctaacaacttacgtcgttatgtcttctccagggaggagct

SEQ ID NO: 287 - DNA, 46 bp, Artificial Sequence
  HL5X-ACTB-834
  source (1)..(46): sequence is synthesized
tagcgctaacaacttacgtcgttatgtccttaatgtcacgcacgat

SEQ ID NO: 288 - DNA, 46 bp, Artificial Sequence
  HL5X-ACTB-603
  source (1)..(46): sequence is synthesized
tagcgctaacaacttacgtcgttatgtggatagcaacgtacatggc

SEQ ID NO: 289 - DNA, 45 bp, Artificial Sequence
  HL5X-ACTB-363
  source (1)..(45): sequence is synthesized
tagcgctaacaacttacgtcgttatgtcgtcgcccacataggaat

SEQ ID NO: 290 - DNA, 47 bp, Artificial Sequence
  HR5X-ACTB-1652
  source (1)..(47): sequence is synthesized
gtgtggacttgggagaggacttatacgtcgagttgaacgtcgtaaca

SEQ ID NO: 291 - DNA, 45 bp, Artificial Sequence
  HR5X-ACTB-1490
  source (1)..(45): sequence is synthesized
atgctcgctccaaccgacttatacgtcgagttgaacgtcgtaaca

SEQ ID NO: 292 - DNA, 47 bp, Artificial Sequence
  HR5X-ACTB-1389
  source (1)..(47): sequence is synthesized
caaataaagccatgccaatcttatacgtcgagttgaacgtcgtaaca

SEQ ID NO: 293 - DNA, 46 bp, Artificial Sequence
  HR5X-ACTB-1272
  source (1)..(46): sequence is synthesized
gccggactcgtcatactccttatacgtcgagttgaacgtcgtaaca

SEQ ID NO: 294 - DNA, 47 bp, Artificial Sequence
  HR5X-ACTB-1176
  source (1)..(47): sequence is synthesized
gctcaggaggagcaatgatcttatacgtcgagttgaacgtcgtaaca

SEQ ID NO: 295 - DNA, 45 bp, Artificial Sequence
  HR5X-ACTB-908
  source (1)..(45): sequence is synthesized
cgtcaggcagctcgtagcttatacgtcgagttgaacgtcgtaaca

SEQ ID NO: 296 - DNA, 46 bp, Artificial Sequence
  HR5X-ACTB-834
  source (1)..(46): sequence is synthesized
ggcgacgtagcacagcttcttatacgtcgagttgaacgtcgtaaca

SEQ ID NO: 297 - DNA, 47 bp, Artificial Sequence
  HR5X-ACTB-603
  source (1)..(47): sequence is synthesized
cgtacagggatagcacagccttatacgtcgagttgaacgtcgtaaca

SEQ ID NO: 298 - DNA, 45 bp, Artificial Sequence
  HR5X-ACTB-363
  source (1)..(45): sequence is synthesized
cctctcttgctctgggccttatacgtcgagttgaacgtcgtaaca

SEQ ID NO: 299 - DNA, 45 bp, Artificial Sequence
  HL5X-ACTB-1533
  source (1)..(45): sequence is synthesized
tagcgctaacaacttacgtcgttatgtcggccacattgtgaactt

SEQ ID NO: 300 - DNA, 49 bp, Artificial Sequence
  HR5X-ACTB-1533
  source (1)..(49): sequence is synthesized
caacaatgtgcaatcaaagtccttatacgtcgagttgaacgtcgtaaca

SEQ ID NO: 301 - DNA, 46 bp, Artificial Sequence
  HL5X-ACTB-1819
  source (1)..(46): sequence is synthesized
tagcgctaacaacttacgtcgttatgacatctcaagttgggggaca

SEQ ID NO: 302 - DNA, 47 bp, Artificial Sequence
  HR5X-ACTB-1839
  source (1)..(47): sequence is synthesized
gggagaccaaaagccttcatttatacgtcgagttgaacgtcgtaaca

SEQ ID NO: 303 - DNA, 44 bp, Artificial Sequence
  HL2X-ACTB-1678
  source (1)..(44): sequence is synthesized
tcgtacgtctaacttacgtcgttatgtcccctgtgtggacttgg

SEQ ID NO: 304 - DNA, 48 bp, Artificial Sequence
  HR2X-ACTB-1678
  source (1)..(48): sequence is synthesized
cacgaaagcaatgctatcaccttatacgtcgagttgaagaacaacctg

SEQ ID NO: 305 - DNA, 46 bp, Artificial Sequence
  HL2X-ACTB-463
  source (1)..(46): sequence is synthesized
tcgtacgtctaacttacgtcgttatgagaaggtgtggtgccagatt

SEQ ID NO: 306 - DNA, 45 bp, Artificial Sequence
  HR2X-ACTB-481
  source (1)..(45): sequence is synthesized
ccacacgcagctcattgtttatacgtcgagttgaagaacaacctg

SEQ ID NO: 307 - DNA, 46 bp, Artificial Sequence
  HL1X-ACTB-463
  source (1)..(46): sequence is synthesized
gacgctaatagacttacgtcgttatgagaaggtgtggtgccagatt

SEQ ID NO: 308 - DNA, 45 bp, Artificial Sequence
  HR1X-ACTB-481
  source (1)..(45): sequence is synthesized
ccacacgcagctcattgtttatacgtcgagttgatagacactctt

SEQ ID NO: 309 - DNA, 50 bp, Artificial Sequence
  HL2X-UBC-2636
  source (1)..(50): sequence is synthesized
tcgtacgtctaacttacgtcgttatgttaaaaggggaaacttagacaccc

SEQ ID NO: 310 - DNA, 50 bp, Artificial Sequence
  HR2X-UBC-2659
  source (1)..(50): sequence is synthesized
gtgcaatgaaatttgttgaaaccttatacgtcgagttgaagaacaacctg

SEQ ID NO: 311 - DNA, 47 bp, Artificial Sequence
  HL2X-UBC-2482
  source (1)..(47): sequence is synthesized
tcgtacgtctaacttacgtcgttatgttggatctttgccttgacatt

SEQ ID NO: 312 - DNA, 47 bp, Artificial Sequence
  HR2X-UBC-2482
  source (1)..(47): sequence is synthesized
ggagggatgccttccttatcttatacgtcgagttgaagaacaacctg

SEQ ID NO: 313 - DNA, 44 bp, Artificial Sequence
  HL6X-UBC-2055
  source (1)..(44): sequence is synthesized
taggtcaggaaacttacgtcgttatgtgctggtcaggagggatg

SEQ ID NO: 314 - DNA, 47 bp, Artificial Sequence
  HR6X-UBC-2055
  source (1)..(47): sequence is synthesized
tcccagcaaagatcaacctcttatacgtcgagttgaatagccaggtt

SEQ ID NO: 315 - DNA, 50 bp, Artificial Sequence
  HL5X-UBC-2637
  source (1)..(50): sequence is synthesized
tagcgctaacaacttacgtcgttatgttaaaaggggaaacttagacaccc

SEQ ID NO: 316 - DNA, 51 bp, Artificial Sequence
  HL5X-UBC-2580
  source (1)..(51): sequence is synthesized
tagcgctaacaacttacgtcgttatgtctttctggatgttgtagtcagaca

SEQ ID NO: 317 - DNA, 46 bp, Artificial Sequence
  HL5X-UBC-2535
  source (1)..(46): sequence is synthesized
tagcgctaacaacttacgtcgttatgtgtttcccagcaaagatcaa

SEQ ID NO: 318 - DNA, 48 bp, Artificial Sequence
  HL5X-UBC-2491
  source (1)..(48): sequence is synthesized
tagcgctaacaacttacgtcgttatgttccttatcttggatctttgcc

SEQ ID NO: 319 - DNA, 44 bp, Artificial Sequence
  HL5X-UBC-2381
  source (1)..(44): sequence is synthesized
tagcgctaacaacttacgtcgttatgtgagacggagcaccaggt

SEQ ID NO: 320 - DNA, 44 bp, Artificial Sequence
  HL5X-UBC-2314
  source (1)..(44): sequence is synthesized
tagcgctaacaacttacgtcgttatgttccagctgtttcccagc

SEQ ID NO: 321 - DNA, 44 bp, Artificial Sequence
  HL5X-UBC-2283
  source (1)..(44): sequence is synthesized
tagcgctaacaacttacgtcgttatgtgctggtcaggagggatg

SEQ ID NO: 322 - DNA, 45 bp, Artificial Sequence
  HL5X-UBC-2232
  source (1)..(45): sequence is synthesized
tagcgctaacaacttacgtcgttatgtcaatggtgtcactcggct

SEQ ID NO: 323 - DNA, 45 bp, Artificial Sequence
  HL5X-UBC-2153
  source (1)..(45): sequence is synthesized
tagcgctaacaacttacgtcgttatgtaagacggagcaccaggtg

SEQ ID NO: 324 - DNA, 49 bp, Artificial Sequence
  HR5X-UBC-2637
  source (1)..(49): sequence is synthesized
tgcaatgaaatttgttgaaaccttatacgtcgagttgaacgtcgtaaca

SEQ ID NO: 325 - DNA, 47 bp, Artificial Sequence
  HR5X-UBC-2580
  source (1)..(47): sequence is synthesized
ggaccaagtgcagagtggacttatacgtcgagttgaacgtcgtaaca

SEQ ID NO: 326 - DNA, 45 bp, Artificial Sequence
  HR5X-UBC-2535
  source (1)..(45): sequence is synthesized
gtgcgtccatcttccagcttatacgtcgagttgaacgtcgtaaca

SEQ ID NO: 327 - DNA, 45 bp, Artificial Sequence
  HR5X-UBC-2491
  source (1)..(45): sequence is synthesized
ctgatcaggagggatgccttatacgtcgagttgaacgtcgtaaca

SEQ ID NO: 328 - DNA, 47 bp, Artificial Sequence
  HR5X-UBC-2381
  source (1)..(47): sequence is synthesized
gaagatttgcatcccacctcttatacgtcgagttgaacgtcgtaaca

SEQ ID NO: 329 - DNA, 45 bp, Artificial Sequence
  HR5X-UBC-2314
  source (1)..(45): sequence is synthesized
agacagggtgcgtccatcttatacgtcgagttgaacgtcgtaaca

SEQ ID NO: 330 - DNA, 47 bp, Artificial Sequence
  HR5X-UBC-2283
  source (1)..(47): sequence is synthesized
tcccagcaaagatcaacctcttatacgtcgagttgaacgtcgtaaca

SEQ ID NO: 331 - DNA, 48 bp, Artificial Sequence
  HR5X-UBC-2232
  source (1)..(48): sequence is synthesized
tggatctttgccttgacattcttatacgtcgagttgaacgtcgtaaca

SEQ ID NO: 332 - DNA, 47 bp, Artificial Sequence
  HR5X-UBC-2153
  source (1)..(47): sequence is synthesized
gaagatctgcatcccacctcttatacgtcgagttgaacgtcgtaaca

SEQ ID NO: 333 - DNA, 46 bp, Artificial Sequence
  HL5X-UBC-2435
  source (1)..(46): sequence is synthesized
tagcgctaacaacttacgtcgttatgtcgagggtgatggtcttacc

SEQ ID NO: 334 - DNA, 45 bp, Artificial Sequence
  HR5X-UBC-2453
  source (1)..(45): sequence is synthesized
gtgtcactgggctccaccttatacgtcgagttgaacgtcgtaaca

SEQ ID NO: 335 - DNA, 50 bp, Artificial Sequence
  HL1X-UBC-2636
  source (1)..(50): sequence is synthesized
gacgctaatagacttacgtcgttatgttaaaaggggaaacttagacaccc

SEQ ID NO: 336 - DNA, 50 bp, Artificial Sequence
  HR1X-UBC-2659
  source (1)..(50): sequence is synthesized
gtgcaatgaaatttgttgaaaccttatacgtcgagttgatagacactctt

SEQ ID NO: 337 - DNA, 46 bp, Artificial Sequence
  HL3X-PPIA-1397
  source (1)..(46): sequence is synthesized
tattcgttcgaacttacgtcgttatgtcaggaggctgaggcactag

SEQ ID NO: 338 - DNA, 47 bp, Artificial Sequence
  HR3X-PPIA-1397
  source (1)..(47): sequence is synthesized
acgcccattatcccagctacttatacgtcgagttgaccgacgtattg

SEQ ID NO: 339 - DNA, 48 bp, Artificial Sequence
  HL5X-PPIA-2201
  source (1)..(48): sequence is synthesized
tagcgctaacaacttacgtcgttatgtcaggtctgagccacaagtaca

SEQ ID NO: 340 - DNA, 46 bp, Artificial Sequence
  HL5X-PPIA-2124
  source (1)..(46): sequence is synthesized
tagcgctaacaacttacgtcgttatgtgagatgcacaagtggtggt

SEQ ID NO: 341 - DNA, 50 bp, Artificial Sequence
  HL5X-PPIA-2028
  source (1)..(50): sequence is synthesized
tagcgctaacaacttacgtcgttatgtctaggataggcaaacttgattgc

SEQ ID NO: 342 - DNA, 46 bp, Artificial Sequence
  HL5X-PPIA-1982
  source (1)..(46): sequence is synthesized
tagcgctaacaacttacgtcgttatgtgttaaggtgggcagagaag

SEQ ID NO: 343 - DNA, 48 bp, Artificial Sequence
  HL5X-PPIA-1883
  source (1)..(48): sequence is synthesized
tagcgctaacaacttacgtcgttatgttagtgggaagggtatcccttt

SEQ ID NO: 344 - DNA, 46 bp, Artificial Sequence
  HL5X-PPIA-1820
  source (1)..(46): sequence is synthesized
tagcgctaacaacttacgtcgttatgtcaaagcctccataaccaaa

SEQ ID NO: 345 - DNA, 47 bp, Artificial Sequence
  HL5X-PPIA-1732
  source (1)..(47): sequence is synthesized
tagcgctaacaacttacgtcgttatgtgcagggagactgactgtagc

SEQ ID NO: 346 - DNA, 49 bp, Artificial Sequence
  HR5X-PPIA-2201
  source (1)..(49): sequence is synthesized
gatgtttatttccaccttgcacttatacgtcgagttgaacgtcgtaaca

SEQ ID NO: 347 - DNA, 50 bp, Artificial Sequence
  HR5X-PPIA-2124
  source (1)..(50): sequence is synthesized
cgtaaccagacaacacacaagacttatacgtcgagttgaacgtcgtaaca

SEQ ID NO: 348 - DNA, 46 bp, Artificial Sequence
  HR5X-PPIA-2028
  source (1)..(46): sequence is synthesized
aatgatcaaatccgccaccttatacgtcgagttgaacgtcgtaaca

SEQ ID NO: 349 - DNA, 49 bp, Artificial Sequence
  HR5X-PPIA-1982
  source (1)..(49): sequence is synthesized
tgggttaagaaccctagaggtcttatacgtcgagttgaacgtcgtaaca

SEQ ID NO: 350 - DNA, 49 bp, Artificial Sequence
  HR5X-PPIA-1883
  source (1)..(49): sequence is synthesized
aatttcctgagaaaccaagtccttatacgtcgagttgaacgtcgtaaca

SEQ ID NO: 351 - DNA, 47 bp, Artificial Sequence
  HR5X-PPIA-1820
  source (1)..(47): sequence is synthesized
ttggtcaggtttgcaaaaccttatacgtcgagttgaacgtcgtaaca

SEQ ID NO: 352 - DNA, 47 bp, Artificial Sequence
  HR5X-PPIA-1732
  source (1)..(47): sequence is synthesized
tagtctgcgccttaaccctcttatacgtcgagttgaacgtcgtaaca

SEQ ID NO: 353 - DNA, 45 bp, Artificial Sequence
  HL5X-PPIA-378
  source (1)..(45): sequence is synthesized
tagcgctaacaacttacgtcgttatgtgggaaccatttgtgttgg

SEQ ID NO: 354 - DNA, 47 bp, Artificial Sequence
  HR5X-PPIA-378
  source (1)..(47): sequence is synthesized
tggcagtgcagatgaaaaacttatacgtcgagttgaacgtcgtaaca

SEQ ID NO: 355 - DNA, 44 bp, Artificial Sequence
  HL5X-PPIA-655
  source (1)..(44): sequence is synthesized
tagcgctaacaacttacgtcgttatgtgcagcgagagcacaaag

SEQ ID NO: 356 - DNA, 47 bp, Artificial Sequence
  HR5X-PPIA-655
  source (1)..(47): sequence is synthesized
acatggaacccaaagggaacttatacgtcgagttgaacgtcgtaaca

SEQ ID NO: 357 - DNA, 48 bp, Artificial Sequence
  HL5X-PPIA-588
  source (1)..(48): sequence is synthesized
tagcgctaacaacttacgtcgttatgtacagaaggaatgatctggtgg

SEQ ID NO: 358 - DNA, 45 bp, Artificial Sequence
  HR5X-PPIA-606
  source (1)..(45): sequence is synthesized
aggggtgctctcctgagcttatacgtcgagttgaacgtcgtaaca

SEQ ID NO: 359 - DNA, 46 bp, Artificial Sequence
  HL2X-PPIA-473
  source (1)..(46): sequence is synthesized
tcgtacgtctaacttacgtcgttatgtccatggcctccacaatatt

SEQ ID NO: 360 - DNA, 43 bp, Artificial Sequence
  HR2X-PPIA-489
  source (1)..(43): sequence is synthesized
cctggacccaaagcgcttatacgtcgagttgaagaacaacctg

SEQ ID NO: 361 - DNA, 46 bp, Artificial Sequence
  HL1X-PPIA-473
  source (1)..(46): sequence is synthesized
gacgctaatagacttacgtcgttatgtccatggcctccacaatatt

SEQ ID NO: 362 - DNA, 43 bp, Artificial Sequence
  HR1X-PPIA-489
  source (1)..(43): sequence is synthesized
cctggacccaaagcgcttatacgtcgagttgatagacactctt

SEQ ID NO: 363 - DNA, 49 bp, Artificial Sequence
  HL5X-SOX4-5611
  source (1)..(49): sequence is synthesized
tagcgctaacaacttacgtcgttatgttaaaaaggcagacgtcctagtg

SEQ ID NO: 364 - DNA, 47 bp, Artificial Sequence
  HR5X-SOX4-5611
  source (1)..(47): sequence is synthesized
aaacccttaacggaactgccttatacgtcgagttgaacgtcgtaaca

SEQ ID NO: 365 - DNA, 46 bp, Artificial Sequence
  HL5X-SOX4-492
  source (1)..(46): sequence is synthesized
tagcgctaacaacttacgtcgttatgtctggtgcaaaatctgttcc

SEQ ID NO: 366 - DNA, 49 bp, Artificial Sequence
  HR5X-SOX4-492
  source (1)..(49): sequence is synthesized
gcattggaataaagaatcagccttatacgtcgagttgaacgtcgtaaca

SEQ ID NO: 367 - DNA, 45 bp, Artificial Sequence
  HL5X-SOX4-4150
  source (1)..(45): sequence is synthesized
tagcgctaacaacttacgtcgttatgtgcccctgtaccacatcaa

SEQ ID NO: 368 - DNA, 45 bp, Artificial Sequence
  HR5X-SOX4-4169
  source (1)..(45): sequence is synthesized
cactcgccctccactgacttatacgtcgagttgaacgtcgtaaca

SEQ ID NO: 369 - DNA, 47 bp, Artificial Sequence
  HL5X-SOX4-1521
  source (1)..(47): sequence is synthesized
tagcgctaacaacttacgtcgttatgtctctccctctctctcgctct

SEQ ID NO: 370 - DNA, 46 bp, Artificial Sequence
  HR5X-SOX4-1521
  source (1)..(46): sequence is synthesized
tcccaggctggagagtctcttatacgtcgagttgaacgtcgtaaca

SEQ ID NO: 371 - DNA, 46 bp, Artificial Sequence
  HL5X-SOX4-2074
  source (1)..(46): sequence is synthesized
tagcgctaacaacttacgtcgttatgtgtctttgagcagcttccag

SEQ ID NO: 372 - DNA, 47 bp, Artificial Sequence
  HR5X-SOX4-2074
  source (1)..(47): sequence is synthesized
aatgaaagggatcttgtcgcttatacgtcgagttgaacgtcgtaaca

SEQ ID NO: 373 - DNA, 46 bp, Artificial Sequence
  HL5X-SOX4-3011
  source (1)..(46): sequence is synthesized
tagcgctaacaacttacgtcgttatgtcaaagtttgagctggggtt

SEQ ID NO: 374 - DNA, 45 bp, Artificial Sequence
  HR5X-SOX4-3011
  source (1)..(45): sequence is synthesized
ctgcccagggacatgctcttatacgtcgagttgaacgtcgtaaca

SEQ ID NO: 375 - DNA, 43 bp, Artificial Sequence
  HL5X-SOX4-3450
  source (1)..(43): sequence is synthesized
tagcgctaacaacttacgtcgttatgtcttcctcctccgcgtc

SEQ ID NO: 376 - DNA, 44 bp, Artificial Sequence
  HR5X-SOX4-3450
  source (1)..(44): sequence is synthesized
gtcgcccctgtctacccttatacgtcgagttgaacgtcgtaaca

SEQ ID NO: 377 - DNA, 42 bp, Artificial Sequence
  HL5X-SOX4-3809
  source (1)..(42): sequence is synthesized
tagcgctaacaacttacgtcgttatgtcagtcccggcgttcc

SEQ ID NO: 378 - DNA, 44 bp, Artificial Sequence
  HR5X-SOX4-3809
  source (1)..(44): sequence is synthesized
cgcctgcgtggagtctcttatacgtcgagttgaacgtcgtaaca

SEQ ID NO: 379 - DNA, 46 bp, Artificial Sequence
  HL5X-SOX4-4009
  source (1)..(46): sequence is synthesized
tagcgctaacaacttacgtcgttatgttccagttcgtgtcctcctc

SEQ ID NO: 380 - DNA, 46 bp, Artificial Sequence
  HR5X-SOX4-4009
  source (1)..(46): sequence is synthesized
agtttgaccgtgaacccccttatacgtcgagttgaacgtcgtaaca

SEQ ID NO: 381 - DNA, 45 bp, Artificial Sequence
  HL5X-SOX4-5145
  source (1)..(45): sequence is synthesized
tagcgctaacaacttacgtcgttatgtttttacaagacgggggcc

SEQ ID NO: 382 - DNA, 47 bp, Artificial Sequence
  HR5X-SOX4-5145
  source (1)..(47): sequence is synthesized
tttggctaattctcctccccttatacgtcgagttgaacgtcgtaaca

SEQ ID NO: 383 - DNA, 45 bp, Artificial Sequence
  HL2X-SOX4-4169
  source (1)..(45): sequence is synthesized
tcgtacgtctaacttacgtcgttatgtgcccctgtaccacatcaa

SEQ ID NO: 384 - DNA, 45 bp, Artificial Sequence
  HR2X-SOX4-4169
  source (1)..(45): sequence is synthesized
cactcgccctccactgacttatacgtcgagttgaagaacaacctg

SEQ ID NO: 385 - DNA, 49 bp, Artificial Sequence
  HL2X-SOX4-5611
  source (1)..(49): sequence is synthesized
tcgtacgtctaacttacgtcgttatgttaaaaaggcagacgtcctagtg

SEQ ID NO: 386 - DNA, 47 bp, Artificial Sequence
  HR2X-SOX4-5611
  source (1)..(47): sequence is synthesized
aaacccttaacggaactgccttatacgtcgagttgaagaacaacctg

SEQ ID NO: 387 - DNA, 46 bp, Artificial Sequence
  HL2X-SOX4-3908
  source (1)..(46): sequence is synthesized
tcgtacgtctaacttacgtcgttatgtcctcccctcaacatctcct

SEQ ID NO: 388 - DNA, 44 bp, Artificial Sequence
  HR2X-SOX4-3908
  source (1)..(44): sequence is synthesized
ggtcacactggctggccttatacgtcgagttgaagaacaacctg

SEQ ID NO: 389 - DNA, 46 bp, Artificial Sequence
  HL2X-SOX4-1449
  source (1)..(46): sequence is synthesized
tcgtacgtctaacttacgtcgttatgtgagtctggagaccgtgcta

SEQ ID NO: 390 - DNA, 46 bp, Artificial Sequence
  HR2X-SOX4-1469
  source (1)..(46): sequence is synthesized
cagtttgctgtctctcggcttatacgtcgagttgaagaacaacctg

SEQ ID NO: 391 - DNA, 42 bp, Artificial Sequence
  HL2X-SOX4-1933
  source (1)..(42): sequence is synthesized
tcgtacgtctaacttacgtcgttatgtcggggtcttgcacca

SEQ ID NO: 392 - DNA, 45 bp, Artificial Sequence
  HR2X-SOX4-1933
  source (1)..(45): sequence is synthesized
gtcgcttgatgtgcccacttatacgtcgagttgaagaacaacctg

SEQ ID NO: 393 - DNA, 43 bp, Artificial Sequence
  HL2X-ID2-107
  source (1)..(43): sequence is synthesized
tcgtacgtctaacttacgtcgttatgtagctgcgcttggcacc

SEQ ID NO: 394 - DNA, 42 bp, Artificial Sequence
  HR2X-ID2-107
  source (1)..(42): sequence is synthesized
tgccgcctgctgagcttatacgtcgagttgaagaacaacctg

SEQ ID NO: 395 - DNA, 47 bp, Artificial Sequence
  HL2X-ID2-224
  source (1)..(47): sequence is synthesized
tcgtacgtctaacttacgtcgttatgtgtttttcctaacggacctca

SEQ ID NO: 396 - DNA, 44 bp, Artificial Sequence
  HR2X-ID2-224
  source (1)..(44): sequence is synthesized
gctgtggtccgacaggcttatacgtcgagttgaagaacaacctg

SEQ ID NO: 397 - DNA, 43 bp, Artificial Sequence
  HL2X-ID2-284
  source (1)..(43): sequence is synthesized
tcgtacgtctaacttacgtcgttatgtcatcgggtcgtccaca

SEQ ID NO: 398 - DNA, 49 bp, Artificial Sequence
  HR2X-ID2-284
  source (1)..(49): sequence is synthesized
tcgttcatgttgtatagcaggcttatacgtcgagttgaagaacaacctg

SEQ ID NO: 399 - DNA, 47 bp, Artificial Sequence
  HL2X-ID2-411
  source (1)..(47): sequence is synthesized
tcgtacgtctaacttacgtcgttatgtgcaggtccaagatgtagtcg

SEQ ID NO: 400 - DNA, 43 bp, Artificial Sequence
  HR2X-ID2-411
  source (1)..(43): sequence is synthesized
cgagtccagggcgatcttatacgtcgagttgaagaacaacctg

SEQ ID NO: 401 - DNA, 45 bp, Artificial Sequence
  HL2X-ID2-854
  source (1)..(45): sequence is synthesized
tcgtacgtctaacttacgtcgttatgtccccatggtgggaatagt

SEQ ID NO: 402 - DNA, 49 bp, Artificial Sequence
  HR2X-ID2-873
  source (1)..(49): sequence is synthesized
cttgtgattttaacgttttcgcttatacgtcgagttgaagaacaacctg

SEQ ID NO: 403 - DNA, 43 bp, Artificial Sequence
  HL5X-ID2-149
  source (1)..(43): sequence is synthesized
tagcgctaacaacttacgtcgttatgtggctgccctgaagctc

SEQ ID NO: 404 - DNA, 43 bp, Artificial Sequence
  HR5X-ID2-149
  source (1)..(43): sequence is synthesized
gagaccgggagggagcttatacgtcgagttgaacgtcgtaaca

SEQ ID NO: 405 - DNA, 46 bp, Artificial Sequence
  HR5X-ID2-190
  source (1)..(46): sequence is synthesized
acctcacgggactgaaggcttatacgtcgagttgaacgtcgtaaca

SEQ ID NO: 406 - DNA, 46 bp, Artificial Sequence
  HL5X-ID2-197
  source (1)..(46): sequence is synthesized
tagcgctaacaacttacgtcgttatgtgaaggctttcatgctgacc

SEQ ID NO: 407 - DNA, 46 bp, Artificial Sequence
  HL5X-ID2-362
  source (1)..(46): sequence is synthesized
tagcgctaacaacttacgtcgttatgtcaccttcttgttctggggg

SEQ ID NO: 408 - DNA, 47 bp, Artificial Sequence
  HR5X-ID2-362
  source (1)..(47): sequence is synthesized
ctgcaggatttccatcttgcttatacgtcgagttgaacgtcgtaaca

SEQ ID NO: 409 - DNA, 42 bp, Artificial Sequence
  HL5X-ID2-468
  source (1)..(42): sequence is synthesized
tagcgctaacaacttacgtcgttatgtgcccgggtctctggt

SEQ ID NO: 410 - DNA, 45 bp, Artificial Sequence
  HR5X-ID2-468
  source (1)..(45): sequence is synthesized
gtcctggacgcctggttcttatacgtcgagttgaacgtcgtaaca

SEQ ID NO: 411 - DNA, 47 bp, Artificial Sequence
  HL5X-ID2-552
  source (1)..(47): sequence is synthesized
tagcgctaacaacttacgtcgttatgtcagaagggaattcagaagcc

SEQ ID NO: 412 - DNA, 51 bp, Artificial Sequence
  HR5X-ID2-552
  source (1)..(51): sequence is synthesized
gctttgctgtcatttgacattaacttatacgtcgagttgaacgtcgtaaca

SEQ ID NO: 413 - DNA, 48 bp, Artificial Sequence
  HL5X-ID2-593
  source (1)..(48): sequence is synthesized
tagcgctaacaacttacgtcgttatgttattcagccacacagtgcttt

SEQ ID NO: 414 - DNA, 48 bp, Artificial Sequence
  HR5X-ID2-593
  source (1)..(48): sequence is synthesized
aaaagaaatcatgaacaccgcttatacgtcgagttgaacgtcgtaaca

SEQ ID NO: 415 - DNA, 48 bp, Artificial Sequence
  HL5X-ID2-694
  source (1)..(48): sequence is synthesized
tagcgctaacaacttacgtcgttatgtccttgtgaaatggttgaaaaa

SEQ ID NO: 416 - DNA, 49 bp, Artificial Sequence
  HR5X-ID2-694
  source (1)..(49): sequence is synthesized
aaaaggtccattcaacttgtccttatacgtcgagttgaacgtcgtaaca

SEQ ID NO: 417 - DNA, 46 bp, Artificial Sequence
  HL5X-ID2-951
  source (1)..(46): sequence is synthesized
tagcgctaacaacttacgtcgttatgtggtattcacgctccacctt

SEQ ID NO: 418 - DNA, 51 bp, Artificial Sequence
  HR5X-ID2-951
  source (1)..(51): sequence is synthesized
aagtgactgaatactggatccttcttatacgtcgagttgaacgtcgtaaca

SEQ ID NO: 419 - DNA, 48 bp, Artificial Sequence
  HL5X-ID2-1233
  source (1)..(48): sequence is synthesized
tagcgctaacaacttacgtcgttatgtctgaaataaagcaggcaatca

SEQ ID NO: 420 - DNA, 50 bp, Artificial Sequence
  HR5X-ID2-1255
  source (1)..(50): sequence is synthesized
aataaaaatcaaagcactggtccttatacgtcgagttgaacgtcgtaaca

SEQ ID NO: 421 - DNA, 47 bp, Artificial Sequence
  HL5X-ID2-411
  source (1)..(47): sequence is synthesized
tagcgctaacaacttacgtcgttatgtgcaggtccaagatgtagtcg

SEQ ID NO: 422 - DNA, 43 bp, Artificial Sequence
  HR5X-ID2-411
  source (1)..(43): sequence is synthesized
cgagtccagggcgatcttatacgtcgagttgaacgtcgtaaca

SEQ ID NO: 423 - DNA, 47 bp, Artificial Sequence
  HL2X-CTNNB1-1615
  source (1)..(47): sequence is synthesized
tcgtacgtctaacttacgtcgttatgtctataccacccacttggcag

SEQ ID NO: 424 - DNA, 47 bp, Artificial Sequence
  HR2X-CTNNB1-1615
  source (1)..(47): sequence is synthesized
ggacagtacgcacaagagccttatacgtcgagttgaagaacaacctg

SEQ ID NO: 425 - DNA, 46 bp, Artificial Sequence
  HL2X-CTNNB1-2599
  source (1)..(46): sequence is synthesized
tcgtacgtctaacttacgtcgttatgtgattgctgtcacctggagg

SEQ ID NO: 426 - DNA, 47 bp, Artificial Sequence
  HR2X-CTNNB1-2599
  source (1)..(47): sequence is synthesized
cagtatcaaaccaggccagcttatacgtcgagttgaagaacaacctg

SEQ ID NO: 427 - DNA, 46 bp, Artificial Sequence
  HL2X-CTNNB1-2732
  source (1)..(46): sequence is synthesized
tcgtacgtctaacttacgtcgttatgtaaaccactcccaccctacc

SEQ ID NO: 428 - DNA, 49 bp, Artificial Sequence
  HR2X-CTNNB1-2732
  source (1)..(49): sequence is synthesized
tgtggcagatttacaaatagccttatacgtcgagttgaagaacaacctg

SEQ ID NO: 429 - DNA, 44 bp, Artificial Sequence
  HL2X-CTNNB1-3000
  source (1)..(44): sequence is synthesized
tcgtacgtctaacttacgtcgttatgtcgagccctctcagcaac

SEQ ID NO: 430 - DNA, 45 bp, Artificial Sequence
  HR2X-CTNNB1-3000
  source (1)..(45): sequence is synthesized
gagataccagcccaccccttatacgtcgagttgaagaacaacctg

SEQ ID NO: 431 - DNA, 48 bp, Artificial Sequence
  HL2X-CTNNB1-3021
  source (1)..(48): sequence is synthesized
tcgtacgtctaacttacgtcgttatgttggttagtgtgtcaggcactt

SEQ ID NO: 432 - DNA, 48 bp, Artificial Sequence
  HR2X-CTNNB1-3041
  source (1)..(48): sequence is synthesized
tgttcccataggaaactcagcttatacgtcgagttgaagaacaacctg

SEQ ID NO: 433 - DNA, 46 bp, Artificial Sequence
  HL5X-CTNNB1-149
  source (1)..(46): sequence is synthesized
tagcgctaacaacttacgtcgttatgtcagaccttcctccgtctcc

SEQ ID NO: 434 - DNA, 45 bp, Artificial Sequence
  HR5X-CTNNB1-149
  source (1)..(45): sequence is synthesized
ggggactgaagctgctccttatacgtcgagttgaacgtcgtaaca

SEQ ID NO: 435 - DNA, 47 bp, Artificial Sequence
  HL5X-CTNNB1-348
  source (1)..(47): sequence is synthesized
tagcgctaacaacttacgtcgttatgtaacagccgcttttctgtctg

SEQ ID NO: 436 - DNA, 47 bp, Artificial Sequence
  HR5X-CTNNB1-348
  source (1)..(47): sequence is synthesized
agactgttgctgccagtgacttatacgtcgagttgaacgtcgtaaca

SEQ ID NO: 437 - DNA, 46 bp, Artificial Sequence
  HL5X-CTNNB1-958
  source (1)..(46): sequence is synthesized
tagcgctaacaacttacgtcgttatgtcacgatgatgggaaaggtt

SEQ ID NO: 438 - DNA, 47 bp, Artificial Sequence
  HR5X-CTNNB1-958
  source (1)..(47): sequence is synthesized
taaagatggccagtaagcccttatacgtcgagttgaacgtcgtaaca

SEQ ID NO: 439 - DNA, 47 bp, Artificial Sequence
  HL5X-CTNNB1-2117
  source (1)..(47): sequence is synthesized
tagcgctaacaacttacgtcgttatgtctttggatgttttcaatggg

SEQ ID NO: 440 - DNA, 46 bp, Artificial Sequence
  HR5X-CTNNB1-2117
  source (1)..(46): sequence is synthesized
agaggacccctgcagctacttatacgtcgagttgaacgtcgtaaca

SEQ ID NO: 441 - DNA, 46 bp, Artificial Sequence
  HL5X-CTNNB1-2528
  source (1)..(46): sequence is synthesized
tagcgctaacaacttacgtcgttatgtggatagtcagcaccagggt

SEQ ID NO: 442 - DNA, 45 bp, Artificial Sequence
  HR5X-CTNNB1-2528
  source (1)..(45): sequence is synthesized
atctggcagcccatcaacttatacgtcgagttgaacgtcgtaaca

SEQ ID NO: 443 - DNA, 45 bp, Artificial Sequence
  HL5X-CTNNB1-3105
  source (1)..(45): sequence is synthesized
tagcgctaacaacttacgtcgttatgtcctcgaccaaaaaggacc

SEQ ID NO: 444 - DNA, 52 bp, Artificial Sequence
  HR5X-CTNNB1-3105
  source (1)..(52): sequence is synthesized
tcccaaaatccatttgtattgttacttatacgtcgagttgaacgtcgtaaca

SEQ ID NO: 445 - DNA, 48 bp, Artificial Sequence
  HL5X-CTNNB1-3147
  source (1)..(48): sequence is synthesized
tagcgctaacaacttacgtcgttatgttcacttcttgagtcactccca

SEQ ID NO: 446 - DNA, 49 bp, Artificial Sequence
  HR5X-CTNNB1-3147
  source (1)..(49): sequence is synthesized
tgtgatccattcttgtgcattcttatacgtcgagttgaacgtcgtaaca

SEQ ID NO: 447 - DNA, 46 bp, Artificial Sequence
  HL5X-CTNNB1-3618
  source (1)..(46): sequence is synthesized
tagcgctaacaacttacgtcgttatgtgtccaaaacaaggttccaa

SEQ ID NO: 448 - DNA, 50 bp, Artificial Sequence
  HR5X-CTNNB1-3618
  source (1)..(50): sequence is synthesized
gggataaaaggcaactggtaaacttatacgtcgagttgaacgtcgtaaca

SEQ ID NO: 449 - DNA, 45 bp, Artificial Sequence
  HL5X-CTNNB1-2580
  source (1)..(45): sequence is synthesized
tagcgctaacaacttacgtcgttatgtgattgctgtcacctggag

SEQ ID NO: 450 - DNA, 47 bp, Artificial Sequence
  HR5X-CTNNB1-2599
  source (1)..(47): sequence is synthesized
cagtatcaaaccaggccagcttatacgtcgagttgaacgtcgtaaca

SEQ ID NO: 451 - DNA, 44 bp, Artificial Sequence
  HL5X-CTNNB1-3000
  source (1)..(44): sequence is synthesized
tagcgctaacaacttacgtcgttatgtcgagccctctcagcaac

SEQ ID NO: 452 - DNA, 45 bp, Artificial Sequence
  HR5X-CTNNB1-3000
  source (1)..(45): sequence is synthesized
gagataccagcccaccccttatacgtcgagttgaacgtcgtaaca

SEQ ID NO: 453 - DNA, 46 bp, Artificial Sequence
  HL2X-Neg20
  source (1)..(46): sequence is synthesized
tcgtacgtctaacttacgtcgttatgaaccggtacgtacggattga

SEQ ID NO: 454 - DNA, 47 bp, Artificial Sequence
  HR2X-Neg20
  source (1)..(47): sequence is synthesized
aaccggtacgtacggattgattatacgtcgagttgaagaacaacctg

SEQ ID NO: 455 - DNA, 46 bp, Artificial Sequence
  HL5X-Neg20
  source (1)..(46): sequence is synthesized
tagcgctaacaacttacgtcgttatgaaccggtacgtacggattga

SEQ ID NO: 456 - DNA, 47 bp, Artificial Sequence
  HR5X-Neg20
  source (1)..(47): sequence is synthesized
aaccggtacgtacggattgattatacgtcgagttgaacgtcgtaaca

SEQ ID NO: 457 - DNA, 46 bp, Artificial Sequence
  HL4X-SCGB1A1a- 91
  source (1)..(46): sequence is synthesized
ttagtaggcgaacttacgtcgttatgtgcagcagagagccagtgtg

SEQ ID NO: 458 - DNA, 46 bp, Artificial Sequence
  HR4X-SCGB1A1a- 112
  source (1)..(46): sequence is synthesized
ttagtaggcgaacttacgtcgttatgaactggagggtgtgtccatg

SEQ ID NO: 459 - DNA, 47 bp, Artificial Sequence
  HL4X-SCGB1A1b-166
  source (1)..(47): sequence is synthesized
aagttccatggcagcctcatttatacgtcgagttgaacataagtgcg

SEQ ID NO: 460 - DNA, 46 bp, Artificial Sequence
  HR4X-SCGB1A1b-187
  source (1)..(46): sequence is synthesized
ttagtaggcgaacttacgtcgttatgttaatgatgctttctctggg

SEQ ID NO: 461 - DNA, 47 bp, Artificial Sequence
  HL4X-SCGB1A1c-278
  source (1)..(47): sequence is synthesized
gggctattttttccatgagcttatacgtcgagttgaacataagtgcg

SEQ ID NO: 462 - DNA, 47 bp, Artificial Sequence; identical to SEQ ID NO: 461
  HR4X-SCGB1A1c-299
  source (1)..(47): sequence is synthesized

SEQ ID NO: 463 - DNA, 46 bp, Artificial Sequence
  HL4X-SCGB1A1d-374
  source (1)..(46): sequence is synthesized
ttagtaggcgaacttacgtcgttatgtcaaagcatggcagcggcag

SEQ ID NO: 464 - DNA, 46 bp, Artificial Sequence
  HR4X-SCGB1A1d- 395
  source (1)..(46): sequence is synthesized
caaggctggtgggcgtggacttatacgtcgagttgaacataagtgc